(19) United States
(12) Patent Application Publication
(10) Pub. No.: US 2006/0251643 A1
(43) Pub. Date: Nov. 9, 2006

(54) Regulatory zinc finger proteins

(75) Inventors: Jin-Soo Kim, Daejeon (KR); Hyun-Chul Shin, Daejeon (KR); Heung-Sun Kwon, Daejeon (KR)

Correspondence Address: SUGHRUE MION, PLLC, 2100 PENNSYLVANIA AVENUE, N.W., SUITE 800, WASHINGTON, DC, 20037, US

(73) Assignee: TOOLGEN, INC., Daejeon (KR)

(21) Appl. No.: 10/538,041

(22) PCT Filed: Dec. 9, 2003
(86) PCT No.: PCT/KR03/02693

§ 371 Date: Jun. 8, 2005

Related U.S. Application Data
(60) Provisional application No. 60/431,892, filed on Dec. 9, 2002.

(51) Int. Cl. A61K 38/46; C07H 21/04; C12P 21/06; C12N 9/64

(52) U.S. Cl. 424/94.65; 435/69.1; 435/226; 435/320.1; 435/325; 536/23.2

(57) ABSTRACT

Disclosed are chimeric zinc finger proteins that can regulate endogenous genes. Examples of such proteins include proteins that can regulate VEGF-A expression. The proteins and nucleic acid encoding them can be used to modulate angiogenesis.

### CROSS-REFERENCE TO RELATED APPLICATIONS

This application claims priority to U.S. Application Ser. No. 60/431,892, filed on Dec. 9, 2002, the contents of which are incorporated by reference herein.

### TECHNICAL FIELD

This invention relates to DNA-binding proteins such as transcription factors.

### BACKGROUND

Most genes are regulated at the transcriptional level by polypeptide transcription factors that bind to specific DNA sites within in the gene, typically in promoter or enhancer regions. These proteins activate or repress transcriptional initiation by RNA polymerase at the promoter, thereby regulating expression of the target gene. Many transcription factors, both activators and repressors, are modular in structure. Such modules can fold as structurally distinct domains and have specific functions, such as DNA binding, dimerization, or interaction with the transcriptional machinery. Effector domains such as activation domains or repression domains retain their function when transferred to DNA-binding domains of heterologous transcription factors. Brent and Ptashne (1985) Cell 43:729-36; Dawson et al., (1995) Mol. Cell Biol. 15:6923-31. The three-dimensional structures of many DNA-binding domains, including zinc finger domains, homeodomains, and helix-turn-helix domains, have been determined from NMR and X-ray crystallographic data.

Zinc finger domains are one type of structural domain that is modular in function. Zinc finger proteins (ZFPs) can be used to regulate transcription. For example, Kim and Pabo demonstrated that the Zif268 protein efficiently repressed VP16-activated transcription of a target gene when the Zif268 protein was bound near the transcription start site of a target gene. Kim and Pabo (1997) J Biol Chem 272:29795-29800. Liu et al. describe up-regulating VEGF-A using engineered zinc finger proteins constructed by site-specific mutagenesis. Liu et al. (2001). J. Biol. Chem. 276, 11323-11334.

### SUMMARY

In one aspect, the invention features a polypeptide that includes a DNA binding domain and can regulate expression of a gene in a cell, e.g., an eukaryotic cell. In one embodiment, the polypeptide binds to a target DNA site in the gene. The DNA binding domain typically includes at least three zinc finger domains.

In one embodiment, at least one, two, or three of the zinc finger domains are naturally-occurring zinc finger domains. For example, these domains can be identical to zinc finger domains of different naturally occurring proteins, or identical to non-adjacent zinc finger domains from a naturally occurring protein. All the zinc finger domains can be naturally-occurring.

In another embodiment, at least one, two, or three of the zinc finger domains is a variant of a naturally-occurring zinc finger domain, e.g., a domain that differs by between one and four or two and five amino acid residues. The polypeptide may include a combination of naturally-occurring zinc finger domains and variant domains.

The polypeptide may regulate any gene. Regulation can be direct such as when the polypeptide interacts with a target site in the target gene. For example, the gene can be an endogenous gene of a cell (e.g., a gene present in a natural genome), a heterologous gene (e.g., a transgene) or a viral gene. In one embodiment, the endogenous gene encodes a secreted polypeptide or a polypeptide that participates in or regulates production of a secreted factor, e.g., a secreted polypeptide. In one embodiment, the endogenous gene regulates cell proliferation, cell migration, or tissue morphogenesis (e.g., angiogenesis).

In one embodiment, the endogenous gene encodes a polypeptide that regulates hormone synthesis, a hormone, or growth factor. Exemplary growth factors include the VEGF family of growth factors.

VEGF-A is one member of this family. In one embodiment, the polypeptide recognizes a target site in the regulatory region of the VEGF-A gene, e.g., a site located between −950 and +450 of the VEGF-A gene. For example, the polypeptide can recognize a site that is located at

about −680, −677, −671, −668, −665, −633R, −632R, −631, −630, −606, −603, −554, −536, −495, −475, −468, −465, 462, −455, −395R, −394R, −393R, −392, −391R, −385R, −382R, −358R, −314R, −282, −206, −206, −203, −184, −181, −137, −124, −90R, −85, −30, 77, 244R, 283R, 342, 357, 366, 434, 435, or 474R of the human VEGF-A promoter, or a site within 60, 50, 20, 10, 5, or 3 nucleotides of such sites. These nucleotide positions indicate the 5′ most nucleotide of the site from transcriptional initiation site on the upper strand of the promoter, unless the letter “R appears, in which case, the numbering of those positions (with the R designation) indicates the 5′ most nucleotide site on the lower (reverse) strand. For example, F435(−90R) target sequence is 5′:−90 to 3′:−98 on reverse strand (5′: −98 to 3′:−90 position from transcriptional initiation site on upper strand). In one embodiment, the polypeptide competes with a polypeptide having a sequence described herein for binding to its target site in the VEGF-A gene.

In one embodiment, the target site is in a regulatory region of the endogenous gene overlaps with a DNase hypersensitive site, or overlaps with the binding site of an endogenous transcription factor. In another embodiment, the target site is within 700, 500, 300, 200, 50, 20, 10, 5, or 3 basepairs of such a site or region. In one embodiment, the polypeptide binds to the target site with a dissociation constant of no more than 20, 7, 5, 3, 2, 1, 0.5, or 0.05 nM.

In one embodiment, when the polypeptide is in a cell, it is able to alter transcription (e.g., represses or activates) of the endogenous gene at least 1.25, 1.5, 1.7, 1.9, 2.0, 2.5, 5, 10, 20, 50, or 100 fold. The polypeptide may have a similar effect when in a cell in an organism.

In one embodiment, the DNA binding domain includes at least two zinc finger domains listed in a single row of Table 1, Table 2, Table 3, Table 4, or Table 5 or includes at least two zinc finger domains that have identical DNA contacting residues as two zinc finger domains listed in a single row of Table 1, Table 2, Table 3, Table 4, or Table 5.

The polypeptide can further include a transcriptional activation or repression domain. The polypeptide can further include a cell transduction domain, e.g., the HIV tat transduction domain.

In one embodiment, the polypeptide suppresses induction of VEGF-A production by hypoxia in a mammalian cell. The suppression can be, e.g., such that VEGF-A levels are less than 80, 70, 60, 50, 40, 30, 20, 10, 5, 3, 2, 1, or 0.1% of the protein level induced by hypoxia in an otherwise identical cell that lacks the polypeptide

The invention also provides a nucleic acid that includes a sequence that encodes a polypeptide described herein and a cell (e.g., a prokaryotic or eukaryotic, e.g., mammalian cell) that includes the nucleic acid. The cell can express the nucleic acid and produce the polypeptide. In one embodiment, the cell is cultured in vitro. The cell can be immuno-isolated or encapsulated. The invention also provides an organism that includes one or more cells in which the polypeptide is produced and an endogenous gene is regulated by the polypeptide.

In another aspect, the invention features a method of regulating an endogenous gene, the method including: providing a cell that includes a coding nucleic acid encoding an artificial polypeptide that includes at least three zinc finger domains, wherein the polypeptide binds to a target DNA site in an endogenous gene; and expressing the coding nucleic acid in the cell under conditions in which the artificial polypeptide is produced, binds to the target DNA site, and regulates the endogenous gene. In one embodiment, at least two of the zinc finger domains are naturally-occurring zinc finger domains. For example, the two zinc finger domains can be identical to zinc finger domains of different naturally occurring proteins, or can be non-adjacent zinc finger domains from the same naturally occurring protein. In one embodiment, the artificial polypeptide includes a transcriptional activation or repression domain. The endogenous gene can be repressed or activated. In one embodiment, the cell is provided by contacting the cell with a nucleic acid delivery vehicle, e.g., a liposome, virus, or viral particle. In one embodiment, the cell is a cell within an organism, e.g., a mammalian organism. The method can further include, prior to the expressing, introducing the cell into a subject organism, or encapsulating the cell and introducing the encapsulated cell into a subject organism.

Exemplary polypeptides can include at least two or more zinc finger domains, e.g., two, three or four zinc finger domain in a particular row of a table below:

TABLE 1
Exemplary VEGF-A Binding Proteins (A)

| Name | Motifs (Col. 2) | Specific Domains (Col. 3) |
|---|---|---|
| F475 | mQSHR-mRDHT-mRSNR | QSHR2-RDHT-RSNR |
| F121 | mQSHT-mRSHR-mRDHT | QSHT-RSHR-RDHT |
| F435 | mQSHR-mRDHT-mRSHR | QSHR2-RDHT-RSHR |
| F547 | mRSHR-mRDHT-mVSNV | RSHR-RDHT-VSNV |
| F2825 | mQSHV-mRDHR-mRDHT | QSHV-RDHR1-RDHT |

TABLE 2
Exemplary VEGF-A Binding Proteins (B)

| Name | Motifs (Col. 2) | Specific Domains (Col. 3) |
|---|---|---|
| F480 | mRSHR-mRDHT-mRSHR | RSHR-RDHT-RSHR |
| F2828 | mCSNR-mWSNR-mRDHR | CSNR1-WSNR-RDHR1 |
| F625 | mCSNR-mWSNR-mRSHR | CSNR1-WSNR-RSHR |
| F2830 | mDSNR-mWSNR-mRDHR | DSNRa-WSNR-RDHR1 |
| F2838 | mDSNR-mWSNR-mRSHR | DSNRa-WSNR-RSHR |

TABLE 3
Exemplary VEGF-A Binding Proteins (C)

| Name | Motifs (Col. 2) | Specific Domains (Col. 3) |
|---|---|---|
| F109 | mRDER-mQSSR-mQSHT-mRSNR | RDER1-QSSR1-QSHT-RSNR |
| F2604 | mDSAR-mRSNR-mRDHT-mVSSR | DSAR2-RSNR-RDHT-VSSR |
| F2605 | mQSHT-mDSAR-mRSNR-mRDHT | QSHT-DSAR2-RSNR-RDHT |
| F2607 | mRDHT-mVSNV-mQSHT-mDSAR | RDHT-VSNV-QSHT-DSAR2 |
| F2615 | mRSHR-mDSCR-mQSHT-mDSCR | RSHR-DSCR-QSHT-DSCR |
| F2633 | mQSNR-mQSHR-mRDHT-mRSNR | QSNR3-QSHR2-RDHT-RSNR |
| F2634 | mCSNR-mRDHT-mRSNR-mRSHR | CSNR1-RDHT-RSNR-RSHR |
| F2636 | mRSHR-mQSHT-mRSHR-mRDER | RSHR-QSHT-RSHR-RDER1 |
| F2644 | mQSNR-mRSHR-mQSSR-mRSHR | QSNR3-RSHR-QSSR1-RSHR |
| F2646 | mQSHT-mDSCR-mRDHT-mCSNR | QSHT-DSCR-RDHT-CSNR1 |
| F2650 | mQSHT-mWSNR-mRSHR-mWSNR | QSHT-WSNR-RSHR-WSNR |
| F2679 | mVSNV-mRSHR-mRDER-mQSNV | VSNV-RSHR-RDER1-QSNV2 |

TABLE 4
Exemplary VEGF-A Binding Proteins (D)

| Name | Motifs (Col. 2) | Specific Domains (Col. 3) |
|---|---|---|
| F2610 | mRSNR-mRSHR-mRDHT- | RSNR-RSHR-RDHT-RSHR |
| | mRSHR | |
| F2612 | mRSHR-mRDHT-mRSHR- | RSHR-RDHT-RSHR-RDHT |
| | mRDHT | |
| F2638 | mRSNR-mQSHR-mRDHT- | RSNR-QSHR2-RDHT-RSHR |
| | mRSHR | |

TABLE 5
Exemplary VEGF-A Binding Proteins (E)

| Name | Motifs (Col. 2) | Specific Domains (Col. 3) |
|---|---|---|
| F2608 | mRSHR-mRDHT-mVSNV-mQSHT | RSHR-RDHT-VSNV-QSHT |
| F2611 | mRSHR-mRSHR-mWSNR-mRSHR | RSHR-RSHR-WSNR-RSHR |
| F2617 | mRDER-mRSHR-mDSCR-mQSHT | RDER1-RSHR-DSCR-QSHT |
| F2619 | mRSHR-mVSTR-mQSNR-mRDHT | RSHR-VSTR-QSNR3-RDHT |
| F2623 | mQSHT-mRSNR-mWSNR-mRDER | QSHT-RSNR-WSNR-RDER1 |
| F2625 | mQSHT-mWSNR-mRDHT-mRDER | QSHT-WSNR-RDHT-RDER1 |
| F2628 | mVSSR-mWSNR-mRSNR-mVSSR | VSSR-WSNR-RSNR-VSSR |
| F2629 | mQSHR-mVSSR-mWSNR-mRSNR | QSHR2-VSSR-WSNR-RSNR |
| F2630 | mRDER-mQSHR-mVSSR-mWSNR | RDER1-QSHR2-VSSR-WSNR |
| F2635 | mQSHR-mRSNR-mQSHR-mRDHT | QSHR2-RSNR-QSHR2-RDHT |
| F2637 | mRDHT-mRSNR-mRSHR-mWSNR | RDHT-RSNR-RSHR-WSNR |
| F2642 | mRDHT-mRSHR-mCSNR-mRDHT | RDHT-RSHR-CSNR1-RDHT |
| F2643 | mRSHR-mCSNR-mRDHT-mCSNR | RSHR-CSNR1-RDHT-CSNR1 |
| F2648 | mQSSR-mQSHR-mRSNR-mRSNR | QSSR1-QSHR2-RSNR-RSNR |
| F2651 | mVSTR-mQSHT-mWSNR-mRSHR | VSTR-QSHT-WSNR-RSHR |
| F2653 | mVSTR-mQSNR-mRSHR-mQSNR | VSTR-QSNR3-RSHR-QSNR3 |
| F2654 | mQSNR-mRSHR-mQSNR-mVSNV | QSNR3-RSHR-QSNR3-VSNV |
| F2662 | mDSCR-mRDHT-mVSTR-mRDER | DSCR-RDHT-VSTR-RDER1 |
| F2667 | mRSHR-mDSCR-mRDHT-mRSHR | RSHR-DSCR-RDHT-RSHR |
| F2668 | mRSHR-mRSHR-mQSNV-mQSNV | RSHR-RSHR-QSNV2-QSNV2 |
| F2673 | mRDHT-mVSSR-mRDER-mQSSR | RDHT-VSSR-RDER1-QSSR1 |
| F2682 | mRSNR-mQSSR-mQSNR-mRSHR | RSNR-QSSR1-QSNR3-RSHR |
| F2689 | mRSNR-mDSAR-mQSNR-mQSHT | RSNR-DSAR2-QSNR3-QSHT |
| F2697 | mRSHR-mCSNR-mQSHT-mRSNR | RSHR-CSNR1-QSHT-RSNR |
| F2699 | mRSNR-mQSHT-mDSAR-mRSHR | RSNR-QSHT-DSAR2-RSHR |
| F2703 | mQSHR-mRSHR-mRDER-mRSHR | QSHR2-RSHR-RDER1-RSHR |
| F2702 | mRSHR-mQSHR-mRSHR-mQSNV | RSHR-QSHR2-RSHR-QSNV2 |

In one aspect, the invention features a polypeptide that includes a DNA binding domain. The DNA binding domain has a plurality of zinc finger domains. The polypeptide can alter the expression or production of VEGF-A in cells. For example, the polypeptide can alter the normal response of the cells to a signal that would increase or decrease VEGF-A production or expression. In one embodiment, the polypeptide can suppresses induction of VEGF-A production or expression in cells under conditions in which VEGF-A production or expression is induced. For example, the suppression can have a magnitude such that VEGF-A protein or mRNA levels are less than 80, 70, 60, 50, 40, 30, 20, 10, 5, 3, 2, 1, or 0.5% of the level induced by the condition in otherwise identical cell that lacks the polypeptide. In one embodiment, the condition includes hypoxia.

These conditions can be determined with particularity in human embryonic kidney 293F cell, e.g., as described in the examples below.

The polypeptide can be used in a wide variety of implementations, e.g., in a human cell in culture or in an organism, e.g., in a human or non-human mammalian organism.

In one embodiment, the polypeptide binds to a site in the human VEGF-A gene. In another embodiment, the polypeptide functions indirectly, e.g., it binds to a site in another gene.

In one embodiment, the polypeptide includes a repression domain. The polypeptide can include other features described herein. The invention also features a composition, e.g., a pharmaceutical composition that includes the polypeptide or a nucleic acid encoding the polypeptide.

The composition can be administered to a subject, e.g., in an amount effective to reduce angiogenesis in the subject, e.g., in the vicinity of a lesion in the subject (e.g., a neoplasm) or throughout the subject. In one embodiment, the subject is a human that has or is suspected of having a metastatic cancer.

With respect to any featured polypeptide, the polypeptide can further include a heterologous sequence, e.g., a nuclear localization signal, a small molecular binding domain (e.g., a steroid binding domain), an epitope tag or purification handle, a catalytic domain (e.g., a nucleic acid modifying domain, a nucleic acid cleavage domain, or a DNA repair catalytic domain), a transcriptional function domain (e.g., an activation domain, a repression domain, and so forth), a protein transduction domain (e.g., from HIV tat), and/or a regulatory site (e.g., a phosphorylation site, ubiquitination site, or protease cleavage site).

The polypeptide can be formulated in a pharmaceutical composition, e.g., with one or more additional components. The composition or polypeptide can be included in a kit that also includes another agent or instructions for use, e.g., therapeutic use.

The polypeptide can be attached (covalently or non-covalently) to a solid support, e.g., a bead, matrix, or planar array. The polypeptide can also be attached to a label such as a radioactive compound, a fluorescent compound, another detectable entity, or a component of a detection system (e.g., a chemiluminescent agent).

The invention also includes an isolated nucleic acid that includes a sequence encoding one of the aforementioned polypeptides. The nucleic acid can further include an operably linked regulatory sequence, e.g., a promoter, a transcriptional enhancer, a 5′ untranslated region, a 3′ untranslated region, a virus packaging sequence, and/or a selectable marker. The nucleic acid can be packaged in a virus, e.g., a virus that can infect a mammalian cell, e.g., a lentivirus, retrovirus, pox virus, or adenovirus.

The invention further provides a cell that contains the polypeptide or the nucleic acid that includes a sequence encoding the polypeptide. The cell can be within a tissue in a subject organism or in culture. The cell can be an animal (e.g., mammalian, e.g., a human or non-human cell), plant, or microbial (e.g., fungal or bacterial) cell. The cell can be prepared by introducing the polypeptide into the cell or a parent cell or by introducing the nucleic acid into the cell or parent cell. The nucleic acid can be used to produce the polypeptide in the cell.

The invention still further includes a non-human transgenic mammal, e.g., a mouse, rat, pig, rabbit, cow, goat, or sheep. The genetic complement of the transgenic mammal includes the nucleic acid sequence encoding the chimeric zinc finger polypeptide described above and elsewhere herein. The invention also includes method of producing the polypeptide, e.g., by expressing the nucleic acid, and of using the polypeptide, e.g., to regulate endogenous genes or viral genes in a cell.

With respect to any polypeptide herein that regulates VEGF-A, the polypeptide can be used in a method of regulating VEGF-A expression in a cell. The method includes introducing the polypeptide or a nucleic acid that includes a sequence encoding the polypeptide into a cell. For example, the polypeptide can be introducing using a liposome or by fusion to a protein transduction domain. A nucleic acid can be introduction, e.g., by transfection or viral delivery.

The invention also features a composition, e.g., a pharmaceutical composition that includes a polypeptide that regulates VEGF-A, e.g., as described herein, or a nucleic acid encoding the polypeptide. In one embodiment, the polypeptide can suppress VEGF-A expression and the composition can be administered to a subject, e.g., in an amount effective to reduce angiogenesis in the subject, e.g., in the vicinity of a lesion in the subject (e.g., a neoplasm) or throughout the subject. In one embodiment, the subject is a human that has or is suspected of having a metastatic cancer.

In another embodiment, the polypeptide can increase VEGF-A expression, and the composition is administered to a subject, in an amount effective to increase angiogenesis in the subject. For example, increased angiogenesis can required for, e.g., vascular formation, embryonic development, somatic growth, differentiation of nerve system, maintenance of pregnancy, wound healing etc. The vascular endothelial growth factor (VEGF-A), one of endothelial cell specific growth factor, is s a key factor that regulates endothelial cell growth and differentiation.

Insufficient levels of VEGF or its VEGF₁₆₄ and VEGF₁₈₈ isoform lead to post-natal angiogenesis and ischemic heart disease. Activation of VEGF-A can be used for the treatment or prevention of peripheral artery disease and coronary artery disease. For example, the subject can be a human that has or is suspected of having a wound (internal or external), pregnancy, a neurological problem, an embryonic developmental problem, a cardiovascular disease (e.g., ischemic heart disease, peripheral artery disease, or coronary artery disease).

At least 5 isoforms of VEGF-A protein are produced from different splice variants. These isoforms have different effects on angiogenesis. The activation of VEGF-A by a zinc finger protein, e.g., a protein described herein, may, in some implementations, enable upregulation of entire or particular splice variants that are important for a desired clinical outcome. For example, the zinc finger protein may modulate expression all splice variants, or it may modulate expression of a subset of splice variants, e.g., at least one splice variant.

In another aspect, the invention features an encapsulated composition that includes an encapsulation layer composed of a biocompatible material, and recombinant mammalian cells, wherein the cells contain a nucleic acid comprising a sequence encoding a chimeric zinc finger protein that regulates production of a factor, e.g., secreted factor or a non-secreted protein, e.g., a cytoplasmic protein. In one embodiment, the biocompatible material is permeable at least to proteins having a molecular weight of 10, 20, 30, or 40 kDa. The biocompatible material can retain proteins larger than, e.g., 50, 100, 120, or 200 kDa.

The invention also provides a rapid and scalable cell-based method for identifying and constructing chimeric proteins, e.g., transcription factors. Such transcription factors can be used, for example, for altering the expression of endogenous genes in biomedical and bioengineering applications. Activity of the transcription factors can be assayed in vivo and in cultured cells, e.g., in intact, living cells in culture.

In yet another aspect, the invention features a method of characterizing a chimeric zinc finger protein, e.g., a zinc finger protein described herein. The method includes: introducing a nucleic acid that encodes the protein into a cell; expressing the nucleic acid; and evaluating expression of a target gene. For example, the evaluating can include determining the profile of expression of endogenous genes in the cell. Such an expression profile includes a plurality of values, wherein each value corresponds to the level of expression of a different gene, splice-variant or allelic variant of a gene (i.e., mRNA level) or the abundance of a translation product (i.e., protein level). The value can be a qualitative or quantitative assessment of the level of expression of the gene or the translation product of the gene, i.e., an assessment of the abundance of 1) an mRNA transcribed from the gene, or 2) the polypeptide encoded by the gene.

In yet another aspect, the invention features a method of identifying a chimeric zinc finger protein that can bind to a particular target site. The method includes: providing data records, each record associating an identifier for a naturally-occurring zinc finger domain (e.g., a human zinc finger domain) and at least one 3- or 4-basepair subsite that is recognized by the zinc finger domain referenced by the identifier; parsing the target site into at least two 3- or 4-basepair subsites; for each of the subsites, retrieving a set of the identifiers from the data records, the set comprising identifiers for the zinc finger domains that recognize the subsite; and designing a polypeptide that comprises a zinc finger domain for each of the subsites, the zinc finger domain being referenced by an identifier from the set for the respective subsite.

The data records can include a record that identifies a zinc finger domain of interest. The method can further include the step of synthesizing a nucleic acid that encodes the polypeptide and/or synthesizing the polypeptide in vitro. The method can also include the step of assessing the binding of the polypeptide to the target site, e.g., using an in vitro binding assay or an in vivo assay such as an assay for target gene expression. The synthesized polypeptide can further include an activation or repression domain.

In one embodiment, the method further includes assessing the ability of the polypeptide to alter the expression of one or more endogenous genes. The assessing can include profiling the expression of multiple endogenous genes, e.g., using nucleic acid microarrays, or a single or limited number of genes. The method can also further include contacting the polypeptide with a DNA that includes the target site, e.g., in vitro.

In another embodiment, the method further includes retrieving a nucleic acid encoding the polypeptide from an addressed library of nucleic acids, each nucleic acid of the library including a sequence encoding first and second zinc finger domains.

In another aspect, the invention features certain polypeptides and isolated nucleic acids. A polypeptide of the invention can include, for example, one, two, three, or four zinc finger domains and be related to a reference polypeptide that has a particular amino acid sequence provided herein. For example, the polypeptide can have the same DNA-contacting residues in one, two, three, four or more zinc finger domains as the DNA-contacting residues in respective zinc finger domains of the reference polypeptide. In another example, in three zinc finger domains of the polypeptide, at least 9, 10, or 11 of the DNA-contacting residues (3×4) are identical to the DNA-contacting residues of respective zinc finger domains in the reference polypeptide. In another example, in four zinc finger domains of the polypeptide, at least 12, 13, 14, or 15 of the DNA-contacting residues (4×4) are identical to the DNA-contacting residues of respective zinc finger domains in the reference polypeptide. The polypeptide can be able to bind to the same site as the reference polypeptide, and regulate the same endogenous gene, e.g., within 0.1 to 10 or 0.5 to 1.5 fold of the activity of the reference polypeptide.

In one embodiment, the amino acid sequences of one or more (e.g., all) of the zinc finger domains are naturally occurring sequences. In one embodiment, the polypeptide is able to regulate a target gene, e.g., an endogenous cellular gene, e.g., the same gene as the reference polypeptide, e.g., VEGF-A.

In addition, purified polypeptides of the invention can have an amino acid sequence at least 50%, 60%, 70%, 80%, 90%, 93%, 95%, 96%, 98%, 99%, or 100% identical to a zinc finger domain described herein. The polypeptides can be identical to a zinc finger domain described herein at the amino acid positions corresponding to the DNA contacting residues of the polypeptide. Alternatively, the polypeptides differ from a zinc finger domain described herein at least one of the residues corresponding to the DNA contacting residues of the polypeptide. For example, one or more zinc finger domains in the polypeptides include a conservative substitution at a DNA contacting residue.

The polypeptides can also differ at least one, two, or three residues, e.g., residues other than a DNA contacting residue. For example, within a given zinc finger domain, the polypeptide may differ by a single amino acid from the amino acid sequences referenced above, or by two, three, or four amino acids from the sequences referenced above. The difference may be due to a conservative substitution as defined herein. In one embodiment, the amino acids differences with respect to the sequences referenced above are located between the second zinc-coordinating cysteine and the −1 DNA contacting position (referring to the numbering system for DNA contacting positions described below).

The comparison of sequences and determination of percent identity between two sequences can be accomplished using a mathematical algorithm. In particular, the percent identity between two amino acid sequences is determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

The purified polypeptides can also include one or more of the following: a heterologous DNA binding domain, a nuclear localization signal, a small molecular binding domain (e.g., a steroid binding domain), an epitope tag or purification handle, a catalytic domain (e.g., a nucleic acid modifying domain, a nucleic acid cleavage domain, or a DNA repair catalytic domain) and/or a transcriptional function domain (e.g., an activation domain, a repression domain, and so forth). In one embodiment, the polypeptide further includes a second zinc finger domain, e.g., a domain having a sequence described herein. For example, the polypeptide can include an array of zinc fingers that include two or more zinc finger domains. In one embodiment, one or more of the domains (e.g., each domain) can have a sequence that conforms to a motif described herein, e.g., mCSNR, mDSAR, mnDSCR, mISNR, mQFNR, mQSHV, mQSNI, mQSNK, mQSNR, mQSNV, mQSSR, mQTHQ, mQTHR, mRDER, mRDHT, mRDKR, mRSHR, mRSNR, mVSNV, mVSSR, mVSTR, mWSNR, mDGNV, mDSNR, and mRDNQ. Further, each domain can have a sequence provided herein. As described below, the small letter “m” prefix indicates that the listed four amino acids represent a motif of DNA contacting residues.

Nucleic acids of the invention include nucleic acids encoding the aforementioned polypeptides. A nucleic acid of the invention can be operably regulated by a heterologous nucleic acid sequence, e.g., an inducible promoter (e.g., a steroid hormone regulated promoter, a small-molecule regulated promoter, or an engineered inducible system such as the tetracycline Tet-On and Tet-Off systems). In one embodiment, the promoter is inducible in a mammalian cell.

As described herein, the polypeptide can be produced in a cell and can regulate a gene in the cell, e.g., an endogenous gene, by binding to a target site, e.g., a site that includes a subsite that the respective zinc finger domain(s) recognizes. The cell can be mammalian cell.

The invention further includes a method of expressing a polypeptide described herein, fused to a heterologous nucleic acid binding domain. The method includes introducing into a cell a nucleic acid encoding the aforementioned fusion protein.

In another aspect, the invention features an encapsulated composition. The composition includes an encapsulation layer composed of a biocompatible material and recombinant mammalian cells. The cells contain a nucleic acid including a sequence encoding a chimeric zinc finger protein that regulates production of another nucleic acid in the cells, e.g., a heterologous nucleic acid or an endogenous nucleic acid. For example, the cells can regulate a gene that encodes a secreted polypeptide or that regulates or participates in the production of a secreted factor, e.g., a secreted polypeptide. In one embodiment, the secreted polypeptide is insulin, an insulin-like growth factor, VEGF-A, HGF, interferon, interleukin, or a fibroblast growth factor.

The encapsulation layer typically is permeable at least to proteins having a molecular weight of 10 kDa, e.g., proteins about 10, 20, 30, 40, 50, or 70 kDa in molecular weight. The encapsulation layer can be impermeable, e.g., to proteins larger than those molecular weights, e.g., larger than 100 kDa. Additional encapsulation layers may be present. The chimeric zinc finger protein can include one or more features described herein.

The term “zinc finger protein” refers to any protein that includes a zinc finger domain. A protein can include one or more polypeptide chains. Exemplary zinc finger proteins include two, three, four, five, six, or more zinc finger domains. Typically the protein is a single chain. However, in some embodiment, the protein can include a plurality of polypeptide chains For example, the protein can be a heterodimeric or homodimeric protein.

The term “base contacting positions,” “DNA contacting positions,” or “nucleic acid contacting positions” refers to the four amino acid positions of zinc finger domains that structurally correspond to the positions of amino acids arginine 73, aspartic acid 75, glutamic acid 76, and arginine 79 of ZIF268.

```
(SEQ ID NO:129)
Glu Arg Pro Tyr Ala Cys Pro Val Glu Ser Cys Asp Arg Arg Phe Ser
  1 5 10 15
Arg Ser Asp Glu Leu Thr Arg His Ile Arg Ile His Thr Gly Gln Lys
                        20 25 30
Pro Phe Gln Cys Arg Ile Cys Met Arg Asn Phe Ser Arg Ser Asp His
                  35 40 45
Leu Thr Thr His Ile Arg Thr His Thr Gly Glu Lys Pro Phe Ala Cys
        50 55 60
Asp Ile Cys Gly Arg Lys Phe Ala Arg Ser Asp Glu Arg Lys Arg His
65  70 75 80
Thr Lys Ile His Leu Arg Gln Lys Asp
                                85
```

These positions are also referred to as positions −1, 2, 3, and 6, respectively. To identify positions in a query sequence that correspond to the base contacting positions, the query sequence is aligned to the zinc finger domain of interest such that the cysteine and histidine residues of the query sequence are aligned with those of finger 3 of Zif268. The ClustalW WWW Service at the European Bioinformatics Institute (Thompson et al. (1994) Nucleic Acids Res. 22:4673-4680) provides one convenient method of aligning sequences.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; a group of amino acids having acidic side chains is aspartic acid and glutamic acid; and a group of amino acids having sulfur-containing side chains is cysteine and methionine. Depending on circumstances, amino acids within the same group may be interchangeable. Some additional conservative amino acids substitution groups are: valine-leucine-isoleucine; phenylalanine-tyrosine; lysine-arginine; alanine-valine; aspartic acid-glutamic acid; and asparagine-glutamine.

The term “heterologous polypeptide” refers either to a polypeptide with a non-naturally occurring sequence (e.g., a hybrid polypeptide) or a polypeptide with a sequence identical to a naturally occurring polypeptide but present in a milieu in which it does not naturally occur. For example, the fusion of two naturally occurring polypeptides that are not fused together in Nature results in a heterologous polypeptide in which one polypeptide is heterologous to the other.

The term “hybrid” refers to a non-naturally occurring polypeptide that comprises amino acid sequences derived from either (i) at least two different naturally occurring sequences; (ii) at least one artificial sequence (i.e., a sequence that does not occur naturally) and at least one naturally occurring sequence; or (iii) at least two artificial sequences (same or different). Examples of artificial sequences include mutants of a naturally occurring sequence and de novo designed sequences.

As used herein, the term “hybridizes under stringent conditions” refers to conditions for hybridization in 6× sodium chloride/sodium citrate (SSC) at 45° C., followed by two washes in 0.2×SSC, 0.1% SDS at 65° C. The invention also features nucleic acids that hybridize under stringent conditions to a nucleic acid described herein or to a nucleic acid encoding a polypeptide described herein.

The term “binding preference” refers to the discriminative property of a polypeptide for selecting one nucleic acid binding site relative to another. For example, when the polypeptide is limiting in quantity relative to two different nucleic acid binding sites, a greater amount of the polypeptide will bind the preferred site relative to the other site in an in vivo or in vitro assay described herein.

As used herein, the “dissociation constant” refers to the equilibrium dissociation constant of a protein (e.g., a zinc finger protein) for binding to a target site of interest. In the case of zinc finger protein that recognizes a target site between 9 and 18 basepairs, the binding is evaluated in the context of a 28-basepair double-stranded DNA. The dissociation constant is determined by gel shift analysis using purified protein that is bound in 20 mM Tris pH 7.7, 120 mM NaCl, 5 mM MgCl₂, 20 μM ZnSO₄, 10% glycerol, 0.1% Nonidet P-40, 5 mM DTT, and 0.10 mg/mL BSA (bovine serum albumin) at room temperature. Additional details are provided in Example 1 and Rebar and Pabo ((1994) Science 263:671-673). Dissociation constants can be less than 10⁻⁶, 10⁻⁷, 10⁻⁸, or 10⁻⁹ M.

One polypeptide (the “competing polypeptide”) can be said to “compete” with another (a polypeptide of interest) for a binding site, if, in an in vitro assay using probe molecules with the target site, the competing polypeptide is present at a concentration no more than 10-fold greater than the concentration of the polypeptide of interest, the number of probe molecules bound by the polypeptide of interest is reduced at least 25%. These experiments are done at about 50 fold the Kd of the polypeptide of interest for the probe molecule.

A given zinc finger domain is said to “bind specifically” to a given 3-base pair DNA site if a chimeric protein that includes fingers 1 and 2 of Zif268 and the given zinc finger domain has an affinity of at least 5 nM for a target site that includes both the given 3-base pair DNA site and the 5-bp sequence, 5′-GGGCG-3′, that is recognized by fingers 1 and 2 of Zif268. The terms “recognize” and “specifically bind” are used interchangeably and refer to the discrimination for a binding site by a zinc finger domain in the above Zif268 fusion assay.

As used herein, “degenerate oligonucleotides” refers to both (a) a population of different oligonucleotides that each encode a particular amino acid sequence, and (b) a single species of oligonucleotide that can anneal to more than one sequence, e.g., an oligonucleotide with an unnatural nucleotide such as inosine.

An “isolated composition” refers to a composition that is removed from at least 90% of at least one component of a cellular sample or reaction mixture from which the isolated composition can be obtained. Compositions produced artificially or naturally can be “compositions of at least” a certain degree of purity if the species or population of species of interests is at least 5, 10, 25, 50, 75, 80, 90, 92, 95, 98, or 99% pure on a weight-weight basis. Any protein or nucleic acid composition described herein can be provided in an isolated form.

The use of zinc finger domains is particularly advantageous. First, the zinc finger structure is capable of recognizing very diverse DNA sequences, but any particular zinc finger can have a high degree of specificity for a particular sequence. Second, the structure of naturally occurring zinc finger proteins is modular. For example, the zinc finger protein Zif268, also called “Egr-1,” is composed of a tandem array of three zinc finger domains. Pavletich and Pabo describe the x-ray crystallographic structure of a fragment of the zinc finger protein Zif268. Pavletich and Pabo (1991) Science 252:809-817. In this structural model, the three Zif268 fingers are complexed with DNA. Each finger independently contacts 3-4 basepairs of the DNA recognition site. High affinity binding is achieved by the cooperative effect of having multiple zinc finger modules in the same polypeptide chain.

The present invention avails itself of all the zinc finger domains present in the human genome, or any other genome. This diverse sampling of sequence space occupied by the zinc finger domain structural fold may have the additional advantages inherent in eons of natural selection. Moreover, by utilizing domains from the host species, a zinc finger protein engineered for a gene therapy application by the methods described herein has a reduced likelihood of being regarded as foreign by the host immune response. It is also possible to use non-naturally occurring zinc finger domains, e.g., variants of human or mammalian zinc finger domains or completely artificial zinc finger domains.

The ability to select a DNA binding domain that recognizes a particular sequence permits the design of novel proteins that specifically regulate a target gene, such an endogenous cellular gene. In many implementations, the proteins have therapeutic or industrial applications. Other applications are also possible.

This disclosure also includes a number of examples that demonstrate, using particular embodiments, that zinc finger proteins generally can be used as a therapeutic for treating cancer. The examples show that zinc finger proteins can function as powerful inhibitors of VEGF-A expression. Since VEGF-A contributes to angiogenesis in tumor tissues, zinc finger proteins that modulate (e.g., inhibit) VEGF-A can be used, e.g., to reduce angiogenesis in and near tumors.

All patents, patent applications, and references cited herein are incorporated by reference in their entirety. The following patent applications: WO 01/60970 (Kim et al.); U.S. Published Applications 2002-0061512, 2003-165997, and 2003-194727, and U.S. Ser. Nos. 10/669,861, 60/431,892 and 60/477,459 are expressly incorporated by reference in their entirety for all purposes. The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A, 1B, and 1C list the nucleic acid sequence (SEQ ID NO: 120) of an exemplary region of the human VEGF-A gene. The region includes the promoter. The sequence is from GENBANK® entry AF095785.1. The transcriptional initiation site is at about nucleotide 2363. The start codon is at about nucleotide 3401.

FIGS. 2A, 2B, 2C, 2D, 2E, and 2F list the nucleic acid sequence (SEQ ID NO:121) of an exemplary region of the human transforming protein (FGF4) gene. The region includes the promoter. The sequence is from GENBANK® entry J02986.1 and AP006345.2 (Homo sapiens genomic DNA, chromosome 11 clone:RP11-186D19, complete sequence). The transcriptional initiation site is at about nucleotide 3731. The start codon is at about nucleotide 3959.

FIGS. 3A, 3B, 3C, 3D, and 3E list the nucleic acid sequence (SEQ ID NO:122) of an exemplary region of the human hepatocyte growth factor (HGF) gene. The region includes the promoter. The sequence is from GENBANK® entry AC004960.1 for Homo sapiens PAC clone RP5-1098B1 from 7q11.23-q21 The transcriptional initiation site is at about nucleotide 4389. The start codon is at about nucleotide 4454.

FIG. 4 is a schematic of the VEGF-A promoter.

FIG. 5A provides schematics of exemplary nucleic acid constructs for expressing zinc finger proteins with KRAB domains.

FIG. 5B provides a schematic of an exemplary luciferase reporter construct that contains the VEGF-A promoter.

### DETAILED DESCRIPTION

Chimeric zinc finger proteins that include at least one zinc finger domain can be used to regulate the expression of genes within cells. Zinc finger protein can include two or more naturally-occurring zinc finger domains. In one set of examples, chimeric zinc finger proteins are used to regulate the VEGF-A gene in a mammalian cell.

Chimeric zinc finger proteins can be obtained by a variety of methods.

In one embodiment, these proteins are designed to recognize a target DNA site. Useful target sites include sites in a regulatory region of the target gene or within 1 kb or 500 bp of a regulatory region of a target gene. For example, the target site can be within 1 kb or 500 bp of the TATA box or transcriptional start site of a gene. One method for designing a zinc finger protein includes parsing target sites into 3 or 4 basepair sequences that can be recognized by an individual zinc finger domain. Then a nucleic acid is constructed which includes a sequence that encodes a protein that has consecutive zinc finger domains corresponding to the parsed elements. A plurality of different nucleic acids that encode candidate proteins is constructed and expressed in a host cell. The expression of the target gene is evaluated to identify one or more of the candidates that is able to regulate expression of the target gene.

In another embodiment, a chimeric zinc finger protein is selected from a library of zinc finger domains based on its phenotypic effect in a cell. For example, a nucleic acid library that encodes random chimeras of zinc finger domains is transformed into mammalian culture cells. Nucleic acids of the library are expressed in the cells. The cells are evaluated for a phenotype of interest, and cells in which the phenotype is altered relative to a control are isolated. The library nucleic acids in such cells are recovered, and the zinc finger protein encoded by such recovered nucleic acids can be further characterized, utilized, or modified.

Zinc Finger Domains

Zinc finger domains are small polypeptide domains of approximately 30 amino acid residues in which there are four residues, either cysteine or histidine, appropriately spaced such that they can coordinate a zinc ion (for reviews, see, e.g., Klug and Rhodes, (1987) Trends Biochem. Sci. 12:464-469(1987); Evans and Hollenberg, (1988) Cell 52:1-3; Payre and Vincent, (1988) FEBS Lett. 234:245-250; Miller et al., (1985) EMBO J. 4:1609-1614; Berg, (1988) Proc. Natl. Acad. Sci. U.S.A. 85:99-102; Rosenfeld and Margalit, (1993) J. Biomol. Struct. Dyn.11:557-570). Hence, zinc finger domains can be categorized according to the identity of the residues that coordinate the zinc ion, e.g., as the Cys₂-His₂ class, the Cys₂-Cys₂ class, the Cys₂-CysHis class, and so forth. The zinc coordinating residues of Cys₂-His₂ zinc fingers are typically spaced as follows:

| | |
|---|---|
| C-X₂₋₅-C-X₃-Xₐ-X₅-ψ-X₂-H-X₃₋₅-H, | (SEQ ID NO:123) |

where ψ (psi) is a hydrophobic residue (Wolfe et al., (1999) Annu. Rev. Biophys. Biomol. Struct. 3:183-212), “X” represents any amino acid, the subscript number indicates the number of amino acids, and a subscript with two hyphenated numbers indicates a typical range of intervening amino acids. In many zinc finger domains, the initial cysteine is preceded by phenylalanine or tyrosine and then a non-cysteine amino acid. Typically, the intervening amino acids fold to form an anti-parallel β-sheet that packs against an α-helix, although the anti-parallel β-sheets can be short, non-ideal, or non-existent. The fold positions the zinc-coordinating side chains so they are in a tetrahedral conformation appropriate for coordinating the zinc ion. The base contacting residues are in the loop region between the pair of metal chelating residues.

For convenience, the primary DNA contacting residues of a zinc finger domain are numbered: −1, 2, 3, and 6 based on the following example:

(SEQ ID NO:124)

−1 1 2 3 4 5 6
C-X₂₋₅-C-X₃-Xₐ-X-R-X-D-E-X_{b}-X-R-H-X₃₋₅-H,

As noted in the example above, the DNA contacting residues are Arg (R), Asp (D), Glu (E), and Arg (R). The above motif can be abbreviated RDER. As used herein, such abbreviation is a shorthand that refers to a particular polypeptide sequence from the second residue preceding the first cysteine (above, initial residue of SEQ ID NO:124) to the ultimate metal-chelating histidine (ultimate residue of SEQ ID NO:124). In the above motif and others, Xₐ is frequently aromatic, and X_{b} is frequently hydrophobic. Where two different sequences have the same motif, a number may be used to indicate each sequence (e.g., RDER1 or RDER2).

In certain contexts where made explicitly apparent, the four-letter abbreviation refers to the motif in general. In other words, the motif specifies the amino acids at positions −1, 2, 3, and 6, while the other positions can be any amino acid, typically, but not necessarily, a non-cysteine amino acid. The small letter “m” before a motif can be used to make explicit that the abbreviation is referring to a motif. For example, mRDER refers to a motif in which R appears at positions −1, D at position 2, E at position 3, and R at position 6.

A zinc finger DNA-binding protein may consist of a tandem array of three or more zinc finger domains.

The zinc finger domain (or “ZFD”) is one of the most common eukaryotic DNA-binding motifs, found in species from yeast to higher plants and to humans. By one estimate, there are at least several thousand zinc finger domains in the human genome alone, possibly at least 4,500. Zinc finger domains can be identified in or isolated from zinc finger proteins. Non-limiting examples of zinc finger proteins include CF2-II; Kruppel; WT1; basonuclin; BCL-6/LAZ-3; erythroid Kruppel-like transcription factor; transcription factors Sp1, Sp2, Sp3, and Sp4; transcriptional repressor YY1; EGR1/Krox24; EGR2/Krox20; EGR3/Pilot; EGR4/AT133; Evi-1; GLI1; GLI2; GLI3; HIV-EP1/ZNF40; HIV-EP2; KR1; Zfx; ZfY; and ZNF7.

An artificial transcription factor can include chimeras of available zinc finger domain. In one embodiment, one or more of the zinc finger domains is naturally occurring. Many exemplary human zinc finger domains are described in US 2002-0061512, US 2003-165997, and U.S. Ser. No. 60/431,892. See also Table 6 below. The binding specificities of each domain, can be used to design a transcription factor with a particular specificity.

TABLE 6
Exemplary Zinc Finger Domains

| | | |
|---|---|---|
| | | SEQ |
| | | ID |
| ZFD | Amino Acid Sequence | NO: |

| | | |
|---|---|---|
| CSNR1 | YKCKQCGKAFGCPSNLRRHGRTH | 1 |
| DSAR2 | YSCGICGKSFSDSSAKRRHCILH | 2 |
| DSCR | YTCSDCGKAFRDKSCLNRHRRTH | 3 |
| QSHR2 | YKCGQCGKFYSQVSHLTRHQKIH | 4 |
| QSHT | YKCEECGKAFRQSSHLTTHKIIH | 5 |
| QSNR3 | YECEKCGKAFNQSSNLTRHKKSH | 6 |
| QSNV2 | YVCSKCGKAFTQSSNLTVHQKIH | 7 |
| QSSR1 | YKCPDCGKSFSQSSSLIRHQRTH | 8 |
| RDER1 | YVCDVEGCTWKFARSDELNRHKKRH | 9 |
| RDHT | FQCKTCQRKFSRSDHLKTHTRTH | 10 |
| RSHR | YKCMECGKAFNRRSHLTRHQRIH | 11 |
| RSNR | YICRKCGRGFSRKSNLIRHQRTH | 12 |
| VSNV | YECDHCGKAFSVSSNLNVHRRIH | 13 |
| VSSR | YTCKQCGKAFSVSSSLRRHETTH | 14 |
| VSTR | YECNYCGKTFSVSSTLIRHQRIH | 15 |
| WSNR | YRCEECGKAFRWPSNLTRHKRIH | 16 |
| QSHV | YECDHCGKSFSQSSHLNVHKRTH | 17 |
| RDHR1 | FLCQYCAQRFGRKDHLTRHMKKS | 18 |
| DSNRa# | YRCKYCDRSFSDSSNLQRHVRNIH | 19 |

#indicates that the domain is not a naturally occurring human domain.

Additional exemplary zinc finger domains include domains with the following motifs:

mCSNR, mDSAR, mDSCR, mISNR, mQFNR, mQSHV, mQSNI, mQSNK, mQSNR, mQSNV, mQSSR, mQTHQ, mQTHR, mRDER, mRDHT, mRDKR, mRSHR, mRSNR, mVSNV, mVSSR, mVSTR, mWSNR, mDGNV, mDSNR, and mRDNQ.

It is also possible to use other types of DNA binding domains, e.g., at least one domain other than a zinc finger domain. The invention utilizes collections of nucleic acid binding domains with differing binding specificities. A variety of protein structures are known to interact nucleic acids with high affinity and high specificity. For reviews of structural motifs which recognize double stranded DNA, see, e.g., Pabo and Sauer (1992) Annu. Rev. Biochem. 61:1053-95; Patikoglou and Burley (1997) Annu. Rev. Biophys. Biomol. Struct. 26:289-325; Nelson (1995) Curr Opin GenetDev. 5:180-9). A few non-limiting examples of nucleic acid binding domains, other than zinc finger domains, include: homeodomains, helix-turn-helix domains, winged helix domains, and helix-loop-helix domains.

Transcription Factor Features

In addition to a DNA-binding domain, a transcription factor may optionally include a regulatory domain, a nuclear localization signal, or other feature described herein.

Activation domains. Transcriptional activation domains that may be used in the present invention include but are not limited to the Gal4 activation domain from yeast and the VP16 domain from herpes simplex virus. The ability of a domain to activate transcription can be validated by fusing the domain to a known DNA binding domain and then determining if a reporter gene operably linked to sites recognized by the known DNA-binding domain is activated by the fusion protein.

An exemplary activation domain is the following domain from p65:

(SEQ ID NO:73)

YLPDTDDRHRIEEKRKRTYETFKSIMKKSPFSGPTDPRPPPRRIAVPSRS
SASVPKPAPQPYPFTSSLSTINYDEFPTMVFPSGQISQASALAPAPPQVL
PQAPAPAPAPAMVSALAQAPAPVPVLAPGPPQAVAPPAPKPTQAGEGTLS
EALLQLQFDDEDLGALLGNSTDPAVFTDLASVDNSEFQQLLNQGIPVAPH
TTEPMLMEYPEAITRLVTAQRPPDPAPAPLGAPGLPNGLLSGDEDFSSIA
DMDFSALLSQ

The sequence of an exemplary Gal4 activation domain is as follows:

(SEQ ID NO:74)

NFNQSGNIADSSLSFTFTNSSNGPNLITTQTNSQALSQPIASSNVHDNFM
NNEITASKIDDGNNSKPLSPGWTDQTAYNAFGITTGMFNTTTMDDVYNYL
FDDEDTPPNPKKEISMAYPYDVPDYAS

In bacteria, activation domain function can be emulated by a domain that recruits a wild-type RNA polymerase alpha subunit C-terminal domain or a mutant alpha subunit C-terminal domain, e.g., a C-terminal domain fused to a protein interaction domain.

Repression domains. If desired, a repression domain instead of an activation domain can be fused to the DNA binding domain. Examples of eukaryotic repression domains include repression domains from Kid, UME6, ORANGE, groucho, and WRPW (see, e.g., Dawson et al., (1995) Mol. Cell Biol. 15:6923-31). The ability of a domain to repress transcription can be validated by fusing the domain to a known DNA binding domain and then determining if a reporter gene operably linked to sites recognized by the known DNA-binding domain is repressed by the fusion protein.

A first exemplary repression domain is the “KRAB” domain from the Kid protein (Witzgall R. et al. (1994) Proc, Natl. Acad. Sci. U.S.A., 91(10): 4514-8):

(SEQ ID NO:75)

VSVTFEDVAVLFTRDEWKKLDLSQRSLYREVMLENYSNLASMAGFLFTKP
KVISLLQQGEDPW

A second exemplary repression domain is the KOX repression domain. This domain includes the “KRAB” domain from the human Kox1 protein (Zinc finger protein 10; NCBI protein database AAH24182; GI:18848329), i.e., amino acids 2-97 of Kox1:

(SEQ ID NO:72)

DAKSLTAWSRTLVTFKDVFVDFTREEWKLLDTAQQIVYRNVMLENYKNLV
SLGYQLTKPDVILRLEKGEEPWLVEREIHQETHPDSETAFEIKSSV

A third exemplary repression domain is the following domain from UME6 protein:

(SEQ ID NO:119)

NSASSSTKLDDDLGTAAAVLSNMRSSPYRTHDKPISNVNDMNNTNALGVP
ASRPHSSSFPSKGVLRPILLRIHNSEQQPIFESNNSTACI

The WRPW domain is still another example of a repression domain.

Still other chimeric transcription factors include neither an activation or repression domain. Rather, such transcription factors may alter transcription by displacing or otherwise competing with a bound endogenous transcription factor (e.g., an activator or repressor).

Other Functional Domains. Examples of other functional domains include a histone modifying enzyme (e.g., a histone acetylase or deacetylase), a DNA modifying enzyme (e.g., a methylase), and so forth.

A protein transduction domain can be fused to the zinc finger protein. Protein transduction domains result in uptake of the transduction domain and attached polypeptide into cells. A “protein transduction domain” or “PTD” is an amino acid sequence that can cross a biological membrane, particularly a cell membrane. When attached to a heterologous polypeptide, a PTD can enhance the translocation of the heterologous polypeptide across a biological membrane. The PTD is typically covalently attached (e.g., by a peptide bond) to the heterologous DNA binding domain. For example, the PTD and the heterologous DNA binding domain can be encoded by a single nucleic acid, e.g., in a common open reading frame or in one or more exons of a common gene. An exemplary PTD can include between 10-30 amino acids and may form an amphipathic helix. Many PTD's are basic in character, e.g., include at least 4, 5, 6 or 8 basic residues (e.g., arginine or lysine). A PTD may be able to enhance the translocation of a polypeptide into a cell that lacks a cell wall or a cell from a particular species, e.g., a eukaryotic cell, e.g., a vertebrate cell, e.g., a mammalian cell, such as a human, simian, murine, bovine, equine, feline, or ovine cell.

Typically a PTD is linked to a zinc finger protein by producing the DNA binding domain of the zinc finger protein and the PTD as a single polypeptide chain, but other methods of for physically associating a PTD can be used. For example, the PTD can be associated by a non-covalent interaction (e.g., using biotin-avidin, coiled-coils, etc.) More typically, a PTD can be linked to a zinc finger protein, for example, using a flexible linker. Flexible linkers can include one or more glycine residues to allow for free rotation. For example, the PTD can be spaced from a DNA binding domain of the transcription factor by at least 10, 20, or 50 amino acids. A PTD can be located N- or C-terminal relative to a DNA binding domain.

An zinc finger protein can also include a plurality of PTD's, e.g., a plurality of different PTD's or at least two copies of one PTD.

Exemplary PTD's include the following segments from the antennapedia protein, the herpes simplex virus VP22 protein and HIV TAT protein.

Tat. The Tat protein from Human Immunodeficiency virus type I (HIV-1) has the remarkable capacity to enter cells when added exogenously (Frankel A. D. and Pabo C. O. (1988) Cell 55:1189-1193, Mann D. A and Frankel A. D. (1991) EMBO J. 10:1733-1739, Fawell et al. (1994) Proc. Natl. Acad. Sci. USA 91:664-668). The minimal Tat PTD includes residues 47-57 of the human immunodeficiency virus Tat protein. This peptide sequence is referred to as “TAT” herein.

Antennapedia. The antennapedia homeodomain also includes a peptide that is a PTD. Derossi et al. (1994) J. Bio. Chem. 269:10444-10450. This peptide, also referred to as “Penetratin.”,

VP22. The HSV VP22 protein also includes a PTD. This PTD is located at the VP22 C-terminal 34 amino acid residues. See, e.g., Elliott and O'Hare (1997) Cell 88:223-234 and U.S. Pat. No. 6,184,038.

Another exemplary PTD is a poly-arginine sequence, e.g., a sequence that includes at least 4, 5, 6 or 8 arginine residues, e.g., between 5 and 10 arginine residues.

Cell-specific PTD's. Some PTD's are specific for particular cell types or states. One exemplary cell-specific PTD is the Hn1 synthetic peptide described in U.S. Published Application 2002-0102265. Hn1 is internalized by human head and neck squamous carcinoma cells and can be used to target an artificial transcription factor to a carcinoma, e.g., a carcinoma of the head or neck. or closely related sequences. U.S. Published Application 2002-0102265 also describes a general method for using phage display to identify other peptides and proteins which can function as cell specific PTD's. For additional information about PTD's, see also U.S. 2003-0082561; U.S. 2002-0102265; U.S. 2003-0040038; Schwarze et al. (1999) Science 285:1569-1572; Derossi et al. (1996) J. Biol. Chem. 271:18188; Hancock et al. (1991) EMBO J. 10:4033-4039; Buss et al. (1988) Mol. Cell. Biol. 8:3960-3963; Derossi et al. (1998) Trends in Cell Biology 8:84-87; Lindgren et al. (2000) Trends in Pharmacological Sciences 21:99-103; Kilic et al. (2003) Stroke 34:1304-10; Asoh et al. (2002) Proc Natl Acad Sci USA 99(26):17107-12; and Tanaka et al. (2003) J Immunol. 170(3):1291-8.

Design of Novel DNA-Binding Proteins

In one embodiment, a zinc finger protein is rationally designed by mixing and matching characterized zinc finger domains so that each domain recognizes one segment of the target site. Zinc finger domains can be isolated and characterized, e.g., using the methods described in US 2002-0061512 and 2003-165997. The modular structure of zinc finger domains facilitates their rearrangement to construct new DNA-binding proteins. Zinc finger domains in the naturally-occurring Zif268 protein are positioned in tandem along the DNA double helix. Each domain independently recognizes a different 3-4 basepair DNA segment.

A Database of Zinc Finger Domains. The one-hybrid selection system described above can be utilized to identify one or more zinc finger domains for each possible 3- or 4-basepair binding site or a representative number of such binding sites. The results of this process can be accumulated as a series of associations between a zinc finger domain and its preferred 3- or 4-basepair binding site or sites. Examples of such associations are provided in US 2002-0061512 and 2003-165997.

The results can also be stored in a machine as a database, e.g., a relational database, spreadsheet, or text file. Each record of such a database associates a representation of a zinc finger domain and a string indicating the sequence of the one or more preferred binding sites of the domain. The database record can include an indication of the relative affinity of the zinc finger domains that bind each site. In some implementations, the database record can also include information that indicates the physical location of the nucleic acid encoding the particular zinc finger domain. Such a physical location can be, for example, a particular well of a microtitre plate stored in a freezer.

The database can be configured so that it can be queried or filtered, e.g., using a SQL operating environment, a scripting language (such as PERL or a MICROSOFT EXCEL® macro), or a programming language. Such a database would enable a user to identify one or more zinc finger domains that recognizes a particular 3- or 4-basepair binding site. Database and other information such as can be stored on a database server can also be configured to communicate with each device using commands and other signals that are interpretable by the device. The computer-based aspects of the system can be implemented in digital electronic circuitry, or in computer hardware, firmware, software, or in combinations thereof. An apparatus of the invention, e.g., the database server, can be implemented in a computer program product tangibly embodied in a machine-readable storage device for execution by a programmable processor; and method actions can be performed by a programmable processor executing a program of instructions to perform functions of the invention by operating on input data and generating output. One non-limiting example of an execution environment includes computers running WINDOWS XP® or WINDOWS NT 4.0® (Microsoft, Redmond Wash.), LINUX™, or other operating systems.

The zinc finger domains can also be tested in the context of multiple different fusion proteins to verify their specificity. Moreover, particular binding sites for which a paucity of domains is available can be the target of additional selection screens. Libraries for such selections can be prepared by mutagenizing a zinc finger domain that binds a similar yet distinct site. A complete matrix of zinc finger domains for each possible binding site is not essential, as the domains can be staggered relative to the target binding site in order to best utilize the domains available. Such staggering can be accomplished both by parsing the binding site in the most useful 3 or 4 basepair binding sites, and also by varying the linker length between zinc finger domains. In order to incorporate both selectivity and high affinity into the design polypeptide, zinc finger domains that have high specificity for a desired site can be flanked by other domains that bind with higher affinity, but lesser specificity. The in vivo screening methods described in US 2002-0061512 and 2003-165997 can be used to test the in vivo function, affinity, and specificity of an artificially assembled zinc finger protein and derivatives thereof. Likewise, these methods can be used to optimize such assembled proteins, e.g., by creating libraries of varied linker composition, varied zinc finger domain modules, varied zinc finger domain compositions, and so forth.

Parsing a target site. The target 9-bp or longer DNA sequence is parsed into 3- or 4-bp segments. Zinc finger domains are identified (e.g., from a database described above) that recognize each parsed 3- or 4-bp segment. Longer target sequences, e.g., 20 bp to 500 bp sequences, are also suitable targets as 9 bp, 12 bp, and 15 bp subsequences can be identified within them. In particular, subsequences amenable for parsing into sites well represented in the database can serve as initial design targets.

A scoring regime can be used to estimate the probability that a particular chimeric zinc finger protein would recognize the target site in the cell. The scores can be a function of each component finger's affinity for its preferred subsites, its specificity, and its success in previously designed proteins.

Computer Programs. Computer systems and software can be used to access a machine-readable database described above, parse a target site, and output one or more chimeric zinc finger protein designs.

The techniques may be implemented in programs executing on programmable machines such as mobile or stationary computers, and similar devices that each include a processor, a storage medium readable by the processor, and one or more output devices. Each program may be implemented in a high level procedural or object oriented programming language to communicate with a machine system. Some merely illustrative examples of computer languages include C, C++, JAVA™, Fortran, and VISUAL BASIC™.

Each such program may be stored on a storage medium or device, e.g., compact disc read only memory (CD-ROM), hard disk, magnetic diskette, or similar medium or device, that is readable by a general or special purpose programmable machine for configuring and operating the machine when the storage medium or device is read by the computer to perform the procedures described in this document. The system may also be implemented as a machine-readable storage medium, configured with a program, where the storage medium so configured causes a machine to operate in a specific and predefined manner.

The computer system can be connected to an internal or external network. For example, the computer system can receive requests from a remotely located client system, e.g., using HTTP, HTTPS, or XML protocols. The requests can be an identifier for a known target gene or a string representing the sequence of a target nucleic acid. In the former case, the computer system can access a sequence database such as GENBANK® to retrieve the nucleic acid sequence of regulatory regions of the target gene. The sequence of the regulatory region or the directly-received target nucleic acid sequence is then parsed into subsites, and chimeric zinc finger proteins are designed, e.g., as described above.

The system can communicate the results to the remotely located client. Alternatively, the system can control a robot to physically retrieve nucleic acid encoding the chimeric zinc finger proteins. In this implementation, a library of nucleic acids encoding chimeric zinc finger proteins is constructed and stored, e.g., as frozen purified DNA or frozen bacterial strains harboring the nucleic acids. The robot responds to signals from the computer system by accessing specified addresses of the library. The retrieved nucleic acids can then be processed, packaged and delivered to the client. Alternatively, the retrieved nucleic acids can be introduced into cells and assayed. The computer system can then communicate the results of the assay to the client across the network.

Constructing a Protein from Selected Modules. Once a chimeric polypeptide sequence containing multiple zinc finger domains is designed, a nucleic acid sequence encoding the designed polypeptide sequence can be synthesized. Methods for constructing synthetic genes are routine in the art. Such methods include gene construction from custom synthesized oligonucleotides, PCR mediated cloning, and mega-primer PCR. In one example, nucleic acids encoding selected zinc finger domains are serially ligated to form a nucleic acid encoding a chimeric polypeptide. Additional sequences can be joined to the nucleic acid encoding the designed polypeptide sequence. The additional sequence can itself provide regulatory functions or can encode an amino acid sequence with a desired function.

Profiling Regulatory Properties of a Chimeric Zinc Finger Protein

A chimeric zinc finger protein can be characterized to determine its ability to regulate one or more endogenous genes in a cell, e.g., a mammalian cell. Nucleic acid encoding the chimeric zinc finger protein is first fused to a repression or activation domain, and then introduced into a cell of interest. After appropriate incubation and induction of expression of the coding nucleic acid, mRNA is harvested from the cell and analyzed using a nucleic acid microarray.

Nucleic acid microarrays can be fabricated by a variety of methods, e.g., photolithographic methods (see, e.g., U.S. Pat. No. 5,510,270), mechanical methods (e.g., directed-flow methods as described in U.S. Pat. No. 5,384,261), and pin based methods (e.g., as described in U.S. Pat. No. 5,288,514). The array is synthesized with a unique capture probe at each address, each capture probe being appropriate to detect a nucleic acid for a particular expressed gene.

The mRNA can be isolated by routine methods, e.g., including DNase treatment to remove genomic DNA and hybridization to an oligo-dT coupled solid substrate (e.g., as described in Current Protocols in Molecular Biology, John Wiley & Sons, N.Y). The substrate is washed, and the mRNA is eluted. The isolated mRNA is then reversed transcribed and optionally amplified, e.g., by rtPCR, e.g., as described in (U.S. Pat. No. 4,683,202). The nucleic acid can be labeled during amplification or reverse transcription, e.g., by the incorporation of a labeled nucleotide. Examples of preferred labels include fluorescent labels, e.g., red-fluorescent dye Cy5 (Amersham) or green-fluorescent dye Cy3 (Amersham). Alternatively, the nucleic acid can be labeled with biotin, and detected after hybridization with labeled streptavidin, e.g., streptavidin-phycoerythrin (Molecular Probes).

The labeled nucleic acid is then contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids are contacted to an array under hybridization conditions. The array is washed, and then imaged to detect fluorescence at each address of the array.

A general scheme for producing and evaluating profiles is includes detecting hybridization at each address of the array. The extent of hybridization at an address is represented by a numerical value and stored, e.g., in a vector, a one-dimensional matrix, or one-dimensional array. The vector x has a value for each address of the array. For example, a numerical value for the extent of hybridization at a particular address is stored in variable xₐ. The numerical value can be adjusted, e.g., for local background levels, sample amount, and other variations. Nucleic acid is also prepared from a reference sample and hybridized to the same or a different array. The vector y is construct identically to vector x. The sample expression profile and the reference profile can be compared, e.g., using a mathematical equation that is a function of the two vectors. The comparison can be evaluated as a scalar value, e.g., a score representing similarity of the two profiles. Either or both vectors can be transformed by a matrix in order to add weighting values to different genes detected by the array.

The expression data can be stored in a database, e.g., a relational database such as a SQL database (e.g., Oracle or Sybase database environments). The database can have multiple tables. For example, raw expression data can be stored in one table, wherein each column corresponds to a gene being assayed, e.g., an address or an array, and each row corresponds to a sample. A separate table can store identifiers and sample information, e.g., the batch number of the array used, date, and other quality control information.

Genes that are similarly regulated can be identified by clustering expression data to identify coregulated genes. Such cluster may be indicative of a set of genes coordinately regulated by the chimeric zinc finger protein. Genes can be clustered using hierarchical clustering (see, e.g., Sokal and Michener (1958) Univ. Kans. Sci. Bull. 38:1409), Bayesian clustering, k-means clustering, and self-organizing maps (see, Tamayo et al. (1999) Proc. Natl. Acad. Sci. USA 96:2907).

The similarity of a sample expression profile to a reference expression profile (e.g., a control cell) can also be determined, e.g., by comparing the log of the expression level of the sample to the log of the predictor or reference expression value and adjusting the comparison by the weighting factor for all genes of predictive value in the profile.

Additional Features for Designed Transcription Factors

Peptide Linkers. DNA binding domains can be connected by a variety of linkers. The utility and design of linkers are well known in the art. A particularly useful linker is a peptide linker that is encoded by nucleic acid. Thus, one can construct a synthetic gene that encodes a first DNA binding domain, the peptide linker, and a second DNA binding domain. This design can be repeated in order to construct large, synthetic, multi-domain DNA binding proteins. PCT WO 99/45132 and Kim and Pabo ((1998) Proc. Natl. Acad. Sci. USA 95:2812-7) describe the design of peptide linkers suitable for joining zinc finger domains.

Additional peptide linkers are available that form random coil, α-helical or β-pleated tertiary structures. Polypeptides that form suitable flexible linkers are well known in the art (see, e.g., Robinson and Sauer (1998) Proc Natl Acad Sci USA. 95:5929-34). Flexible linkers typically include glycine, because this amino acid, which lacks a side chain, is unique in its rotational freedom. Serine or threonine can be interspersed in the linker to increase hydrophilicity. In additional, amino acids capable of interacting with the phosphate backbone of DNA can be utilized in order to increase binding affinity. Judicious use of such amino acids allows for balancing increases in affinity with loss of sequence specificity. If a rigid extension is desirable as a linker, α-helical linkers, such as the helical linker described in Pantoliano et al. (1991) Biochem. 30:10117-10125, can be used. Linkers can also be designed by computer modeling (see, e.g., U.S. Pat. No. 4,946,778). Software for molecular modeling is commercially available (e.g., from Molecular Simulations, Inc., San Diego, Calif.). The linker is optionally optimized, e.g., to reduce antigenicity and/or to increase stability, using standard mutagenesis techniques and appropriate biophysical tests as practiced in the art of protein engineering, and functional assays as described herein.

For implementations utilizing zinc finger domains, the peptide that occurs naturally between zinc fingers can be used as a linker to join fingers together. A typical such naturally occurring linker is: Thr-Gly-(Glu or Gln)-(Lys or Arg)-Pro-(Tyr or Phe) (SEQ ID NO:125).

Dimerization Domains. An alternative method of linking DNA binding domains is the use of dimerization domains, especially heterodimerization domains (see, e.g., Pomerantz et al (1998) Biochemistry 37:965-970). In this implementation, DNA binding domains are present in separate polypeptide chains. For example, a first polypeptide encodes DNA binding domain A, linker, and domain B, while a second polypeptide encodes domain C, linker, and domain D. An artisan can select a dimerization domain from the many well-characterized dimerization domains. Domains that favor heterodimerization can be used if homodimers are not desired. A particularly adaptable dimerization domain is the coiled-coil motif, e.g., a dimeric parallel or anti-parallel coiled-coil. Coiled-coil sequences that preferentially form heterodimers are also available (Lumb and Kim, (1995) Biochemistry 34:8642-8648). Another species of dimerization domain is one in which dimerization is triggered by a small molecule or by a signaling event. For example, a dimeric form of FK506 can be used to dimerize two FK506 binding protein (FKBP) domains. Such dimerization domains can be utilized to provide additional levels of regulation.

Functional Assays and Uses

Zinc finger proteins can be evaluated using cell-free assays and cellular assays. Examples of cell-free assays include assays in which at least partially purified protein is evaluated for a biochemical property, e.g., DNA binding in vitro. Examples of useful in vitro assays include electrophoretic mobility shift assays (EMSA), DNA footprinting, DNA methylation protection assays, surface plasmon resonance, fluorescence polarization, and fluorescence resonance energy transfer (FRET). Binding and other functional properties can be assayed in cellular assays or in vivo (e.g., in an organism).

For example, domains can be selected to bind to a target site, e.g., to a promoter site of a gene that modulates cell proliferation. By modular assembly, a protein can be designed that includes (1) the selected domains that respectively bind to subsites spanning the target promoter site, and (2) a transcriptional regulatory domain, e.g., an activation domain or a repression domain. In an example in which the protein regulates a gene that modulates cell proliferation and the protein is intended to counteract cell proliferation, the appropriate transcriptional regulatory domain can be chosen depending on whether the gene increases cell proliferation (e.g., a repression domain is selected) or decreases cell proliferation (e.g., an activation domain is selected). In another example, a library encoding random combinations of zinc finger domains is screened to identify a chimeric zinc finger protein that alters a phenotype.

A nucleic acid sequence encoding a chimeric zinc finger protein can be cloned into an expression vector, e.g., an inducible expression vector as described in Kang and Kim, (2000) J Biol Chem 275:8742. The inducible expression vector can include an inducible promoter or regulatory sequence. Non-limiting examples of inducible promoters include steroid-hormone responsive promoters (e.g., ecdysone-responsive, estrogen-responsive, and glutacorticoid-responsive promoters), the tetracyclin “Tet-On” and “Tet-Off” systems, and metal-responsive promoters. The construct can be transfected into tissue culture cells or into embryonic stem cells to generate a transgenic organism as a model subject. The efficacy of the chimeric zinc finger protein can be determined by inducing expression of the protein and assaying cell proliferation of the tissue culture cell or assaying for developmental changes and/or tumor growth in a transgenic animal model. In addition, the level of expression of the gene being targeted can be assayed by routine methods to detect mRNA, e.g., RT-PCR or Northern blots. A more complete diagnostic includes purifying mRNA from cells expressing and not expressing the chimeric zinc finger protein. The two pools of mRNA are used to probe a microarray containing probes to a large collection of genes, e.g., a collection of genes relevant to the condition of interest (e.g., cancer) or a collection of genes identified in the organism's genome. Such an assay is particularly valuable for determining the specificity of the chimeric zinc finger protein. If the protein binds with high affinity but little specificity, it may cause pleiotropic and undesirable effects by affecting expression of genes in addition to the contemplated target. Such effects are revealed by a global analysis of transcripts.

In addition, the chimeric zinc finger protein can be produced in a subject cell or subject organism in order to regulate an endogenous gene. The chimeric zinc finger protein is configured, as described above, to bind to a region of the endogenous gene and to provide a transcriptional activation or repression function. As described in Kang and Kim (supra), the expression of a nucleic acid encoding the chimeric zinc finger protein can be operably linked to a regulatable promoter (e.g., an inducible or suppressible promoter). By modulating the concentration of an agent that can regulate the promoter, e.g., an inducer for the promoter, the expression of the endogenous gene can be regulated in a concentration dependent manner.

The binding site preference of a zinc finger protein can be verified by a biochemical assay such as EMSA, DNase footprinting, surface plasmon resonance, SELEX, or column binding. The substrate for binding can be, e.g., a synthetic oligonucleotide encompassing the target site or a restriction fragment. The assay can also include non-specific DNA as a competitor, or specific DNA sequences as a competitor. Specific competitor DNAs can include the recognition site for DNA binding with one, two, or three nucleotide mutations. Thus, a biochemical assay can be used to measure not only the affinity of a domain for a given site, but also its affinity to the site relative to other sites. Rebar and Pabo, (1994) Science 263:671-673 describe a method of obtaining apparent K_{d} constants for zinc finger domains from EMSA. Exemplary zinc finger proteins have at least 2, 5, 10, 50, 100, or 500 fold preference for a particular recognition site relative to a related site with one, two, or three nucleotide mutations.

A protein or nucleic acid described herein can also be evaluated, e.g., in vitro or in vivo for a biological activity, e.g., ability to modulate a endothelial cell or to modulate angiogenesis.

Endothelial cell proliferation. A protein or nucleic acid can be tested for endothelial proliferation inhibiting activity using a biological activity assay such as the bovine capillary endothelial cell proliferation assay, the chick CAM assay, the mouse corneal assay, and evaluating the effect of the protein or nucleic acid being tested on implanted tumors. The chick CAM assay is described, e.g., by O'Reilly, et al. in “Angiogenic Regulation of Metastatic Growth” Cell, vol. 79 (2), Oct. 21, 1994, pp. 315-328. Briefly, three-day old chicken embryos with intact yolks are separated from the egg and placed in a petri dish. After three days of incubation a methylcellulose disc containing the protein to be tested is applied to the CAM of individual embryos. After 48 hours of incubation, the embryos and CAMs are observed to determine whether endothelial growth has been inhibited. The mouse corneal assay involves implanting a growth factor-containing pellet, along with another pellet containing the suspected endothelial growth inhibitor, in the cornea of a mouse and observing the pattern of capillaries that are elaborated in the cornea.

Angiogenesis. Angiogenesis may be assayed, e.g., using various human endothelial cell systems, such as umbilical vein, coronary artery, or dermal cells. Suitable assays include Alamar Blue based assays (available from Biosource International) to measure proliferation; migration assays using fluorescent molecules, such as the use of Becton Dickinson Falcon HTS FluoroBlock cell culture inserts to measure migration of cells through membranes in presence or absence of angiogenesis enhancer or suppressors; and tubule formation assays based on the formation of tubular structures by endothelial cells on Matrigel™ (Becton Dickinson).

Cell adhesion. Cell adhesion assays measure adhesion of cells to purified adhesion proteins or adhesion of cells to each other, in presence or absence of the protein or nucleic acid being tested. Cell-protein adhesion assays measure the ability of agents to modulate the adhesion of cells to purified proteins. For example, recombinant proteins are produced, diluted to 2.5 g1 mL in PBS, and used to coat the wells of a microtiter plate. The wells used for negative control are not coated. Coated wells are then washed, blocked with 1% BSA, and washed again. Compounds are diluted to 2 times final test concentration and added to the blocked, coated wells. Cells are then added to the wells, and the unbound cells are washed off. Retained cells are labeled directly on the plate by adding a membrane-permeable fluorescent dye, such as calcein-AM, and the signal is quantified in a fluorescent microplate reader.

Cell-cell adhesion assays can be used to measure the ability of the protein or nucleic acid being tested to modulate binding of cells to each other. These assays can use cells that naturally or recombinantly express an adhesion protein of choice. In an exemplary assay, cells expressing the cell adhesion protein are plated in wells of a multiwell plate together with other cells (either more of the same cell type, or another type of cell to which the cells adhere). The cells that can adhere are labeled with a membrane-permeable fluorescent dye, such as BCECF, and allowed to adhere to the monolayers in the presence of the protein or nucleic acid being tested. Unbound cells are washed off, and bound cells are detected using a fluorescence plate reader. High-throughput cell adhesion assays have also been described. See, e.g., Falsey J R et al., Bioconjug Chem. May-June 2001;12(3):346-53.

Tubulogenesis. Tubulogenesis assays can be used to monitor the ability of cultured cells, generally endothelial cells, to form tubular structures on a matrix substrate, which generally simulates the environment of the extracellular matrix. Exemplary substrates include Matrigel™ (Becton Dickinson), an extract of basement membrane proteins containing laminin, collagen IV, and heparin sulfate proteoglycan, which is liquid at 4° C. and forms a solid gel at 37° C. Other suitable matrices comprise extracellular components such as collagen, fibronectin, and/or fibrin. Cells are stimulated with a pro-angiogenic stimulant, and their ability to form tubules is detected by imaging. Tubules can generally be detected after an overnight incubation with stimuli, but longer or shorter time frames may also be used. Tube formation assays are well known in the art (e.g., Jones M K et al., 1999, Nature Medicine 5:1418-1423). These assays have traditionally involved stimulation with serum or with the growth factors FGF or VEGF. In one embodiment, the assay is performed with cells cultured in serum free medium. In one embodiment, the assay is performed in the presence of one or more pro-angiogenic agents, e.g, inflammatory angiogenic factors such as TNF-α, or FGF, VEGF, phorbol myristate acetate (PMA), TNF-alpha, ephrin, etc.

Cell Migration. An exemplary assay for endothelial cell migration is the human microvascular endothelial (HMVEC) migration assay. See, e.g., Tolsma et al. (1993) J. Cell Biol 122, 497-511. Migration assays are known in the art (e.g., Paik J H et al., 2001, J Biol Chem 276:11830-11837). In one example, cultured endothelial cells are seeded onto a matrix-coated porous lamina, with pore sizes generally smaller than typical cell size. The lamina is typically a membrane, such as the transwell polycarbonate membrane (Corning Costar Corporation, Cambridge, Mass.), and is generally part of an upper chamber that is in fluid contact with a lower chamber containing pro-angiogenic stimuli. Migration is generally assayed after an overnight incubation with stimuli, but longer or shorter time frames may also be used. Migration is assessed as the number of cells that crossed the lamina, and may be detected by staining cells with hemotoxylin solution (VWR Scientific.), or by any other method for determining cell number. In another exemplary set up, cells are fluorescently labeled and migration is detected using fluorescent readings, for instance using the Falcon HTS FluoroBlok (Becton Dickinson). While some migration is observed in the absence of stimulus, migration is greatly increased in response to pro-angiogenic factors. The assay can be used to test the effect of the protein or nucleic acid being tested on endothelial cell migration.

Sprouting assay. An exemplary sprouting assay is a three-dimensional in vitro angiogenesis assay that uses a cell-number defined spheroid aggregation of endothelial cells (“spheroid”), embedded in a collagen gel-based matrix. The spheroid can serve as a starting point for the sprouting of capillary-like structures by invasion into the extracellular matrix (termed “cell sprouting”) and the subsequent formation of complex anastomosing networks (Korff and Augustin, 1999, J Cell Sci 112:3249-58). In an exemplary experimental set-up, spheroids are prepared by pipetting 400 human umbilical vein endothelial cells into individual wells of a nonadhesive 96-well plates to allow overnight spheroidal aggregation (Korff and Augustin: J Cell Biol 143: 1341-52, 1998). Spheroids are harvested and seeded in 900 μl of methocel-collagen solution and pipetted into individual wells of a 24 well plate to allow collagen gel polymerization. Test agents are added after 30 min by pipetting 100 μl of 10-fold concentrated working dilution of the test substances on top of the gel. Plates are incubated at 37° C. for 24 h. Dishes are fixed at the end of the experimental incubation period by addition of paraformaldehyde. Sprouting intensity of endothelial cells can be quantitated by an automated image analysis system to determine the cumulative sprout length per spheroid.

Other exemplary assays include: Ferrara and Henzel (1989) Nature 380:439-443; Gospodarowicz et al. (1989) Proc. Natl. Acad. Sci. USA, 86: 7311-7315; and Claffey et al. (1995) Biochim. Biophys. Acta. 1246:1-9.; Leung et al. (1989) Science 246:1306-1309; Rastinejad et al. (1989) Cell 56:345-355; and U.S. Pat. No. 5,840,693. The ability of a composition to modulate ischemia can be evaluated, e.g., using a rat hindlimb ischemia model (see, e.g., Takeshita, S. et al., Circulation (1998) 98: 1261-63.

Targets for Gene Regulation

The target gene can be any gene, e.g., a chromosomal gene or a heterologous gene (e.g., a transgene). The target gene can be selected, e.g., if it is useful to regulate (e.g., increase or decrease) activity of the target gene. For example, a gene required by a pathogen can be repressed, a gene required for cancerous growth can be repressed, a gene poorly expressed or encoding an unstable protein can be activated and overexpressed, a gene that confers stress resistance can be activated, and so forth.

Examples of specific target genes include genes that encode: cell surface proteins (e.g., glycosylated surface proteins), cancer-associated proteins, cytokines, chemokines, peptide hormones, neurotransmitters, cell surface receptors (e.g., cell surface receptor kinases, seven transmembrane receptors, virus receptors and co-receptors, extracellular matrix binding proteins, cell-binding proteins, antigens of pathogens (e.g., bacterial antigens, malarial antigens, and so forth). Additional protein targets include enzymes such as enolases, cytochrome P450s, acyltransferases, methylases, TIM barrel enzymes, isomerases, acyl transferases, and so forth.

Still more examples include: integrins, cell attachment molecules or “CAMs” such as cadherins, selections, N-CAM, E-CAM, U-CAM, I-CAM and so forth); proteases (e.g., subtilisin, trypsin, chymotrypsin; a plasminogen activator, such as urokinase or human tissue-type plasminogen activator); bombesin; factor IX, thrombin; CD-4; platelet-derived growth factor; insulin-like growth factor-I and -II; nerve growth factor; fibroblast growth factor (e.g., aFGF and bFGF); epidermal growth factor (EGF); VEGF (e.g., VEGF-A); transforming growth factor (TGF, e.g., TGF-α and TGF-β; insulin-like growth factor binding proteins; erythropoietin; thrombopoietin; mucins; human serum albumin; growth hormone (e.g., human growth hormone); proinsulin, insulin A-chain insulin B-chain; parathyroid hormone; thyroid stimulating hormone; thyroxine; follicle stimulating hormone; calcitonin; atrial natriuretic peptides A, B or C; leutinizing hormone; glucagon; factor VIII; hemopoietic growth factor; tumor necrosis factor (e.g., TNF-α and TNF-β); enkephalinase; Mullerian-inhibiting substance; gonadotropin-associated peptide; tissue factor protein; inhibin; activin; vascular endothelial growth factor; receptors for hormones or growth factors; rheumatoid factors; osteoinductive factors; an interferon, e.g., interferon-α,β,γ; colony stimulating factors (CSFs), e.g., M-CSF, GM-CSF, and G-CSF; interleukins (ILs), e.g., IL-1, IL-2, IL-3, IL-4, etc.; decay accelerating factor; and immunoglobulins. In some embodiments, the targetgene encodes a protein or other factor (e.g., an RNA) that is associated with a disease, e.g., cancer, an infectious disease, inflammation, or a cardiovascular disease.

In one embodiment, the gene is a human disease gene. For example, the gene can include a mutation that encodes a defective or impaired enzyme or the gene may have a defect in a regulatory sequence (e.g., a transcriptional, translational, or splicing regulatory sequence). A zinc finger protein can be obtained that increases expression of the gene.

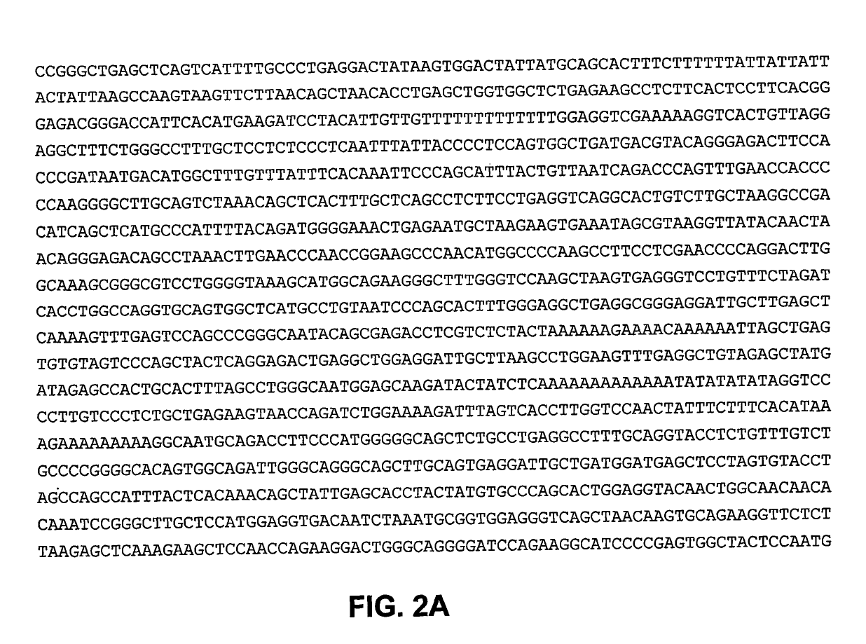

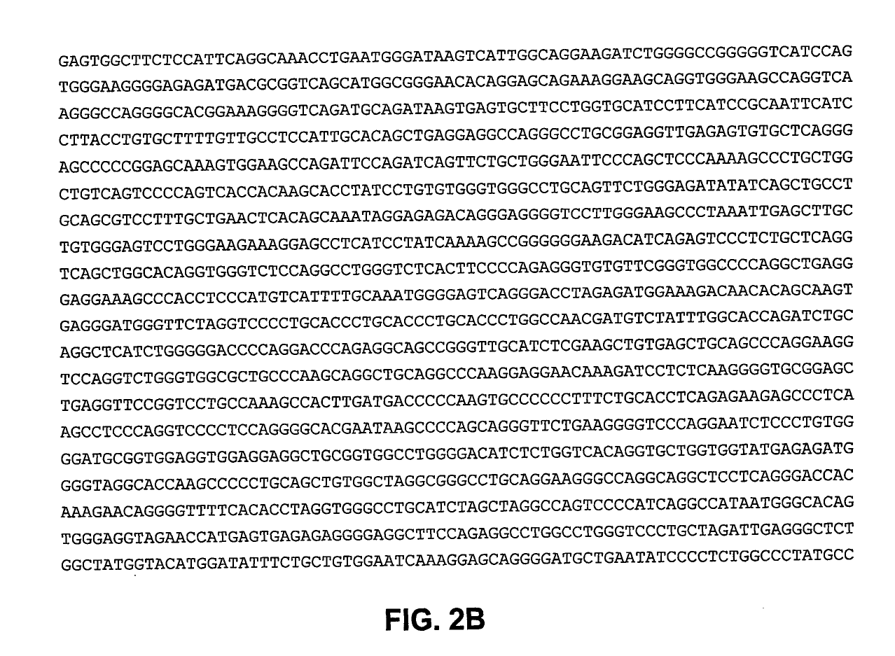

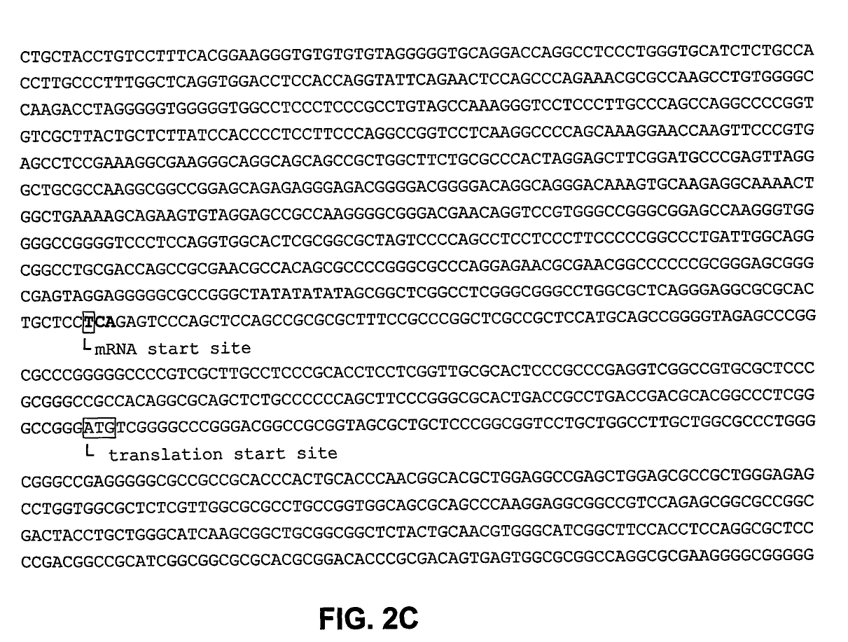

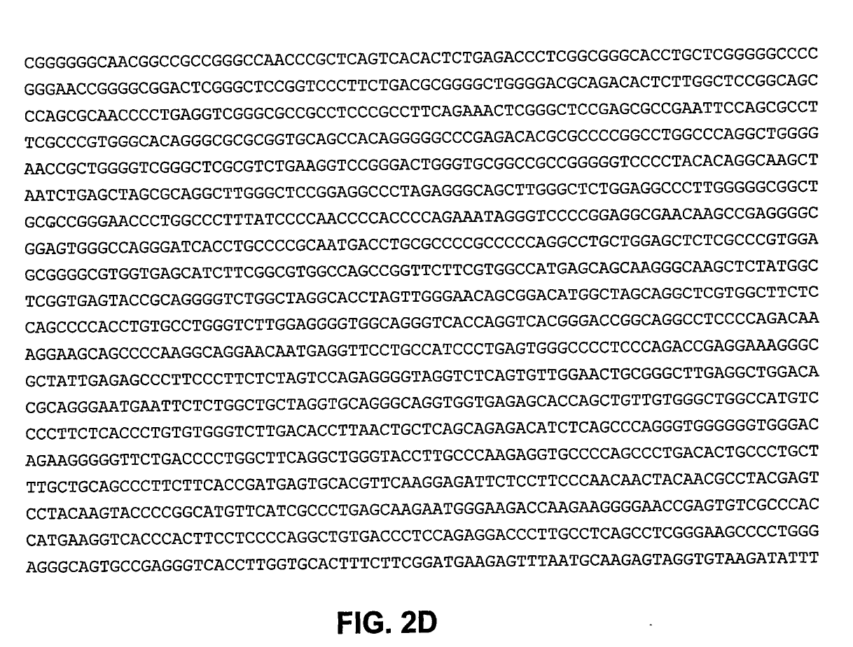

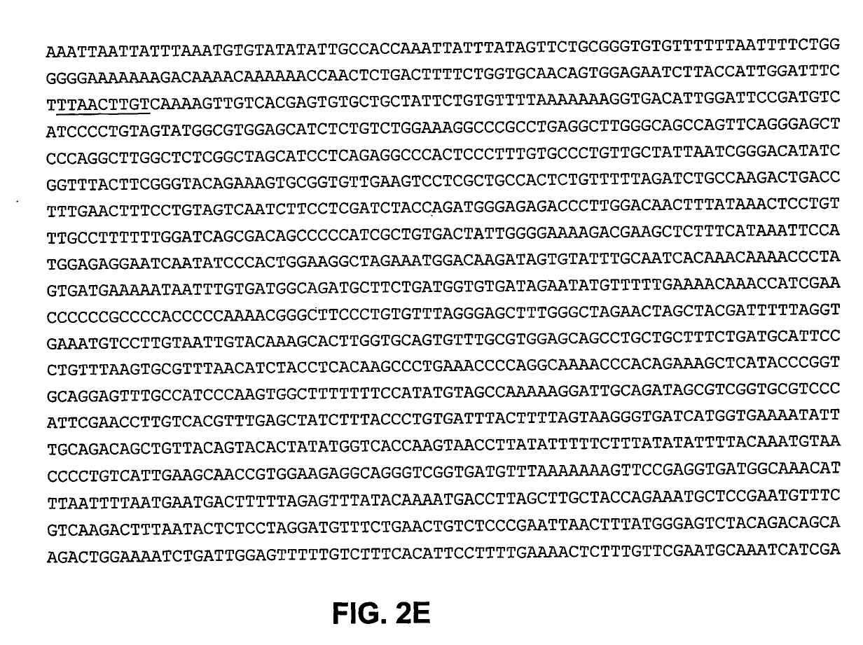

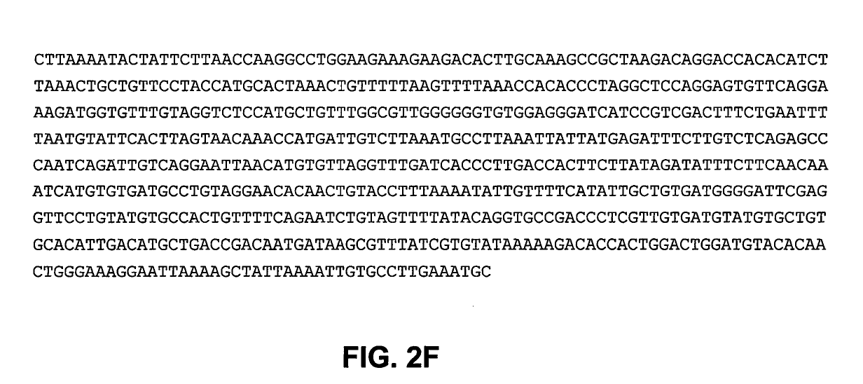

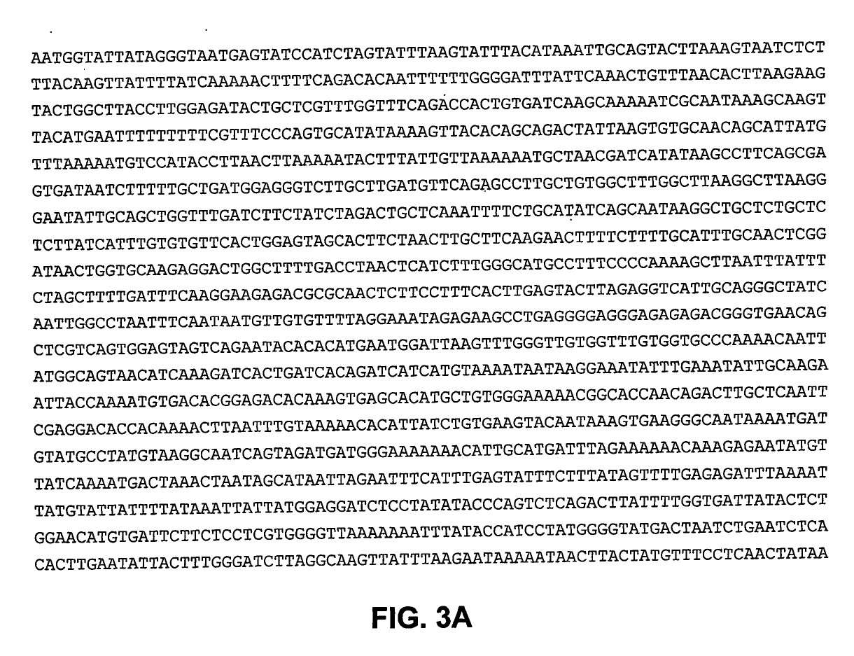

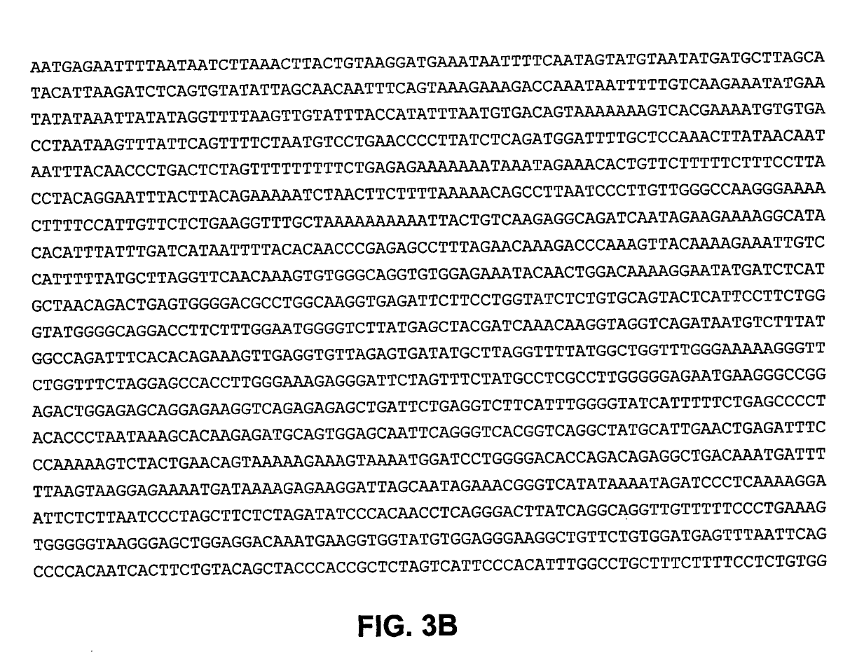

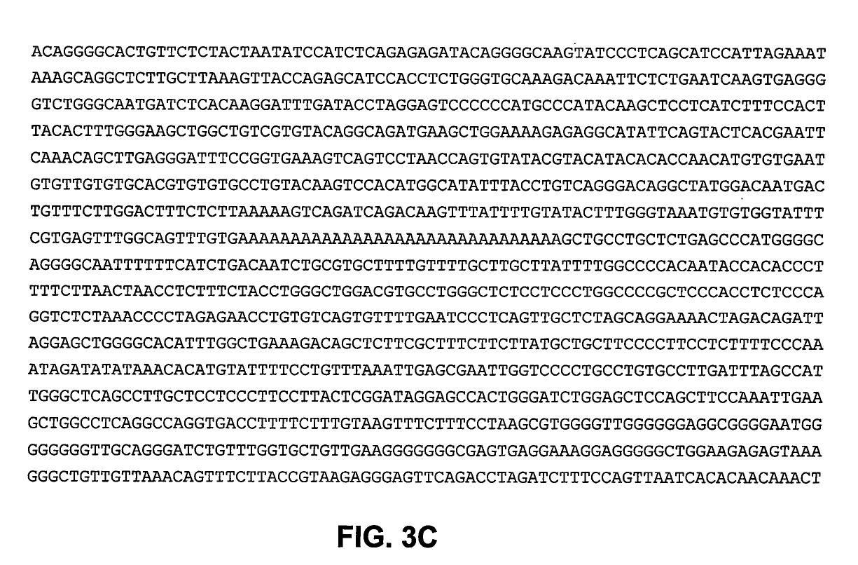

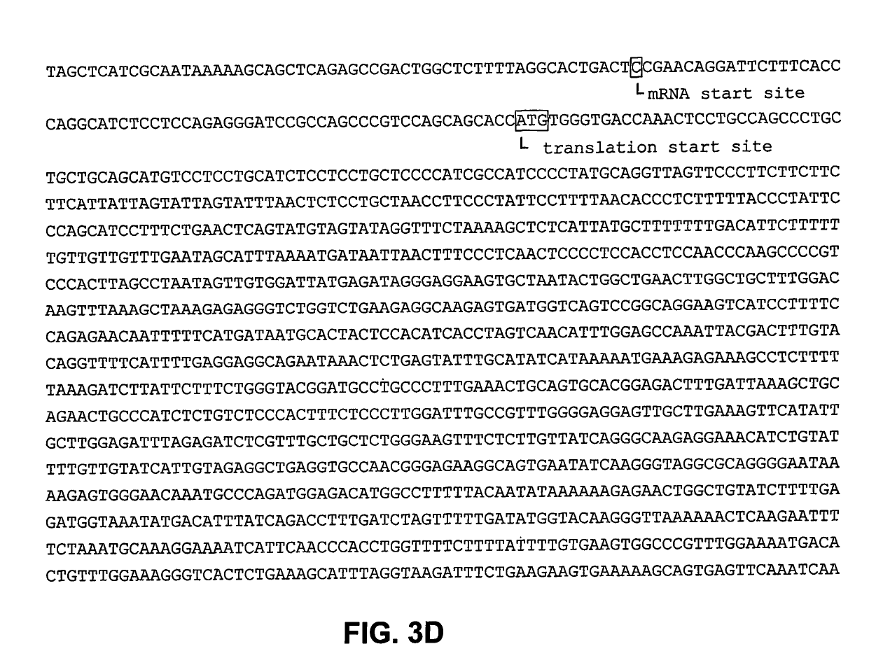

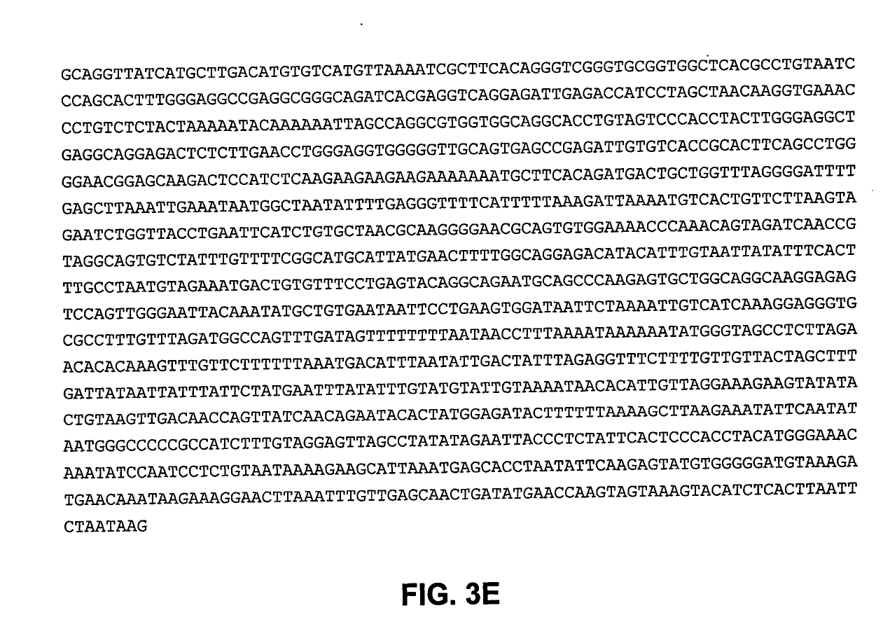

For example, zinc finger proteins can be designed that interact with a FGF gene, e.g., to a binding site in the sequence listed in FIG. 2A-F, or with a hepatocyte growth factor (HGF) gene, e.g., to a binding site in the sequence listed in FIG. 3A-E. For example, the proteins may interact with a promoter region of these genes.

A chimeric zinc finger protein for regulating any gene can be designed to interact with one or more target sites. For example, the target sites can be located in a coding or non-coding region of the gene. In one embodiment, the target site is located in a regulatory region, e.g., a transcriptional regulatory region such as the promoter. In one embodiment, the target site is located within 700, 500, 300, 200, 50, 20, 10, 5, or 3 basepairs of the transcription start site, a Dnase hypersensitive site, or a transcription factor binding site. In an embodiment in which the target gene is VEGF-A, the binding site can differ from (e.g., not overlap with) a site in Table 2 or 3 of WO 02/46412. In another embodiment, the binding site does overlap with such a site.

Gene and Cell-Based Therapeutics

DNA molecules that encode a chimeric zinc finger protein can be inserted into a variety of DNA constructs and vectors for the purposes of gene therapy. As used herein, a “vector” is a nucleic acid molecule competent to transport another nucleic acid molecule to which it has been covalently linked. Vectors include plasmids, cosmids, artificial chromosomes, viral elements, and RNA vectors (e.g., based on RNA virus genomes). The vector can be competent to replicate in a host cell or to integrate into a host DNA. Viral vectors include, e.g., replication defective retroviruses, adenoviruses and adeno-associated viruses.

A gene therapy vector is a vector designed for administration to a subject, e.g., a mammal, such that a cell of the subject is able to express a therapeutic gene contained in the vector. The gene therapy vector can contain regulatory elements, e.g., a 5′ regulatory element, an enhancer, a promoter, a 5′ untranslated region, a signal sequence, a 3′ untranslated region, a polyadenylation site, and a 3′ regulatory region. For example, the 5′ regulatory element, enhancer or promoter can regulate transcription of the DNA encoding the therapeutic polypeptide. The regulation can be tissue specific. For example, the regulation can restrict transcription of the desired gene to brain cells, e.g., cortical neurons or glial cells; hematopoietic cells; or endothelial cells. Alternatively, regulatory elements can be included that respond to an exogenous drug, e.g., a steroid, tetracycline, or the like. Thus, the level and timing of expression of the therapeutic zinc finger protein (e.g., a polypeptide that regulates VEGF) can be controlled.

Gene therapy vectors can be prepared for delivery as naked nucleic acid, as a component of a virus, or of an inactivated virus, or as the contents of a liposome or other delivery vehicle. See, e.g., US 2003-0143266 and 2002-0150626. In one embodiment, the nucleic acid is formulated in a lipid-protein-sugar matrix to form microparticles., e.g., having a diameter between 50 nm to 10 micrometers. The particles may be prepared using any known lipid (e.g., dipalmitoylphosphatidylcholine, DPPC), protein (e.g., albumin), or sugar (e.g., lactose).

The gene therapy vectors can be delivered using a viral system. Exemplary viral vectors include vectors from retroviruses, e.g., Moloney retrovirus, adenoviruses, adeno-associated viruses, and lentiviruses, e.g., Herpes simplex viruses (HSV). HSV, for example, is potentially useful for infecting nervous system cells. See, e.g., US 2003-0147854, 2002-0090716, 2003-0039636, 2002-0068362, and 2003-0104626. The gene delivery agent, e.g., a viral vector, can be produced from recombinant cells which produce the gene delivery system.

A gene therapy vector can be administered to a subject, for example, by intravenous injection, by local administration (see U.S. Pat. No. 5,328,470) or by stereotactic injection (see e.g., Chen et al. (1994) Proc. Natl. Acad. Sci. USA 91:3054-3057). The gene therapy agent can be further formulated, for example, to delay or prolong the release of the agent by means of a slow release matrix. One method of providing a recombinant zinc finger protein, is by inserting a gene therapy vector into bone marrow cells harvested from a subject. The cells are infected, for example, with a retroviral gene therapy vector, and grown in culture. Meanwhile, the subject is irradiated to deplete the subject of bone marrow cells. The bone marrow of the subject is then replenished with the infected culture cells. The subject is monitored for recovery and for production of the therapeutic polypeptide.

Cell based-therapeutic methods include introducing a nucleic acid that encodes the chimeric zinc finger protein operably linked to a promoter into a cell in culture. The chimeric zinc finger protein can be selected to regulate an endogenous gene in the culture cell or to produce a desired phenotype in the cultured cell. Further, it is also possible to modify cells, e.g., stem cells, using nucleic acid recombination, e.g., to insert a transgene, e.g., a transgene encoding a chimeric zinc finger protein that regulates an endogenous gene. The modified stem cell can be administered to a subject. Methods for cultivating stem cells in vitro are described, e.g., in US Application 2002-0081724. In some examples, the stem cells can be induced to differentiate in the subject and express the transgene. For example, the stem cells can be differentiated into liver, adipose, or skeletal muscle cells. The stem cells can be derived from a lineage that produces cells of the desired tissue type, e.g., liver, adipose, or skeletal muscle cells.

In another embodiment, recombinant cells that express or can express a chimeric zinc finger protein, e.g., as described herein, can be used for replacement therapy in a subject. For example, a nucleic acid encoding the chimeric zinc finger protein operably linked to a promoter (e.g., an inducible promoter, e.g., a steroid hormone receptor-regulated promoter) is introduced into a human or nonhuman, e.g., mammalian, e.g., porcine recombinant cell. The cell is cultivated and encapsulated in a biocompatible material, such as poly-lysine alginate, and subsequently implanted into the subject. See, e.g., Lanza (1996) Nat. Biotechnol. 14:1107; Joki et al. (2001) Nat. Biotechnol. 19:35; and U.S. Pat. No. 5,876,742. Other examples of biocompatible polymers for encapsulating cells include sodium alginate, barium alginate or sodium cellulose sulfate. Useful polymers enable proteins (e.g., proteins less than 70, 20, or 10 kDa) to diffuse across them. Ultra-pure materials can improve the viability of encapsulated cells and reduce immunological reactions. Encapsulated cells, e.g., cells that include an artificial transcription factor and can produce a diffusible factor can be used as a therapy in a subject to provide the diffusible factor to the subject.

One exemplary method for encapsulating cells and tissues involves the use of coatings formed of a non-fibrogenic alginate, a gelatinous substance that can be derived from certain kinds of kelp. For example, the cells are suspended in a viscous, liquid alginate, which is then atomized by any of a number of different arrangements into droplets of suitable size to encapsulate the cells. Once the droplets come into contact with a gelling solution, such as calcium chloride or barium chloride, a single layer alginate coating is created around the cells.

Examples of this approach for creating single layer alginate coatings using an electrostatic coating process are shown in U.S. Pat. No. 4,789,550, U.S. Pat. No. 4,956,128, U.S. Pat. No. 5,429,821, U.S. Pat. No. 5,639,467, U.S. Pat. No. 5,656,468 and U.S. Pat. No. 5,693,514. An example for creating a single layer alginate coating using an air knife process is shown in U.S. Pat. No. 5,521,079. A pressurized process for coating droplets is described in U.S. Pat. No. 5,260,002 and U.S. Pat. No. 5,462,866. Other examples for creating a single layer alginate coating using a spinning disk arrangement are shown in U.S. Pat. No. 5,643,594 and U.S. Pat. No. 6,001,387. Examples for creating a single layer alginate coating using a piezoelectric nozzle are shown in U.S. Pat. No. 5,286,496, U.S. Pat. No. 5,648,099 and U.S. Pat. No. 6,033,888. U.S. Pat. No. 5,470,731 and U.S. Pat. No. 5,531,997 describe a double layer coating for tissue that comprises a first layer of a gel-able organic polymer and a cationic polymer and a second water-soluble, semi-permeable layer chemically bonded to the first layer. U.S. Pat. No. 6,020,200 describes a dual layer coating having a stabilized outer layer formed of a cross-linked polymer matrix. U.S. Pat. No. 5,227,298 (Weber at al.) describes a double walled alginate coating.

Encapsulated cells can be implanted by surgery (e.g., laproscopic or conventional surgical methods) or by injection. Cells can be introduced into any appropriate body site including the liver, spleen, thymus, testes, brain, pancreas, lungs, kidneys, peritoneal cavity, subcutaneous tissues, fat pads and other locations. See, e.g., J. Rozga et al., Intraabdominal Organ Transplantation 2000; R. G. Landes Co., USA, 1994: 129.

In implementations where the chimeric zinc finger protein regulates an endogenous gene that encodes a secreted protein, production of the secreted polypeptide can be regulated in the subject by administering an agent (e.g., a steroid hormone) to the subject. In another embodiment, production of the zinc finger protein can be placed under control of an endogenous signal, e.g., a signal indicating reduced level of the secreted protein. Thus, an artificial feedback loop can be used. For example, the signal can be mediated by a transcription factor that is regulated by level of the secreted protein itself.

For additional methods for encapsulating cells, see, for example: U.S. Pat. No. 4,391,909; US 2002-0022016; Lohr et al., (2002) Cancer Chemother Pharmacol, 49: S21-S24; Hobbs et al., (2001) Journal of Investigative Medicine, vol. 49, no.6, 49(6):572-5; Zimmermann et al. (2001) Ann N Y Acad Sci. 2001; Moashebi et al; Tissue Engineering, 2001, vol. 7, 5, 525-534); Orive et al., (2002) Trends in Biotechnology, vol. 20, 382-7; Lim and Sun (1980) Science 210: 908-910; Reed et al. 2001. Nature Biotech. 19:29-34; Domish et al., (2001) “Standards and guidelines for Biopolymers in Tissue-Engineered Medical Products: ASTM Alginate and Chitosan Standard Guides.” Ann N Y Acad Sci. 2001; 944:388-97.

In still another embodiment, the recombinant cells that express or can express a chimeric zinc finger protein are cultivated in vitro. A protein produced by the recombinant cells can be recovered (e.g., purified) from the cells or from media surrounding the cells. In another example the recombinant cells are used as feeder cells.

Pharmaceutical Compositions

In another aspect, the invention provides compositions, e.g., pharmaceutically acceptable compositions, which include an zinc finger protein or a nucleic acid encoding it, e.g., a molecule described herein, formulated together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Exemplary compositions are in the form of injectable or infusible solutions. One mode of administration is parenteral (e.g., intravenous, subcutaneous, intraperitoneal, intramuscular). In one embodiment, the composition that includes the zinc finger protein or a nucleic acid encoding it is administered by intravenous infusion or injection. In another embodiment, the composition that includes the zinc finger protein or a nucleic acid encoding it is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. Endotoxin levels in the preparation can be tested using the Limulus amebocyte lysate assay (e.g., using the kit from Bio Whittaker lot # 7L3790, sensitivity 0.125 EU/mL) according to the USP 24/NF 19 methods. Sterility of pharmaceutical compositions can be determined using thioglycollate medium according to the USP 24/NF 19 methods. For example, the preparation is used to inoculate the thioglycollate medium and incubated at 35° C. for 14 or more days. The medium is inspected periodically to detect growth of a microorganism.

The composition can be formulated as a solution, microemulsion, dispersion, liposome, or other ordered structure suitable to high drug concentration. Sterile injectable solutions can be prepared by incorporating the active compound (i.e., the zinc finger protein or a nucleic acid encoding it) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

A composition that includes a zinc finger protein or a nucleic acid encoding it can be administered by a variety of methods known in the art. For many applications, the route/mode of administration is intravenous injection or infusion. For example, for therapeutic applications, the composition that includes a zinc finger protein or a nucleic acid encoding it can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

In certain embodiments, the composition may be orally administered, for example, with an inert diluent or an assimilable edible carrier. The compound (and other ingredients, if desired) also may be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound described herein by other than parenteral administration, it may be necessary to coat the compound with, or co-administer the compound with, a material to prevent its inactivation.

Pharmaceutical compositions can be administered with medical devices known in the art. For example, in a preferred embodiment, a pharmaceutical composition described herein can be administered with a needle-less hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules also are known.

In certain embodiments, the compounds described herein can be formulated to ensure proper distribution in vivo. For example, the blood-brain barrier (BBB) excludes many highly hydrophilic compounds. To ensure that a therapeutic can cross the BBB (if desired), it can be formulated, for example, in a liposome. For methods of manufacturing liposomes, see, e.g., U.S. Pat. Nos. 4,522,811; 5,374,548; and 5,399,331. The liposomes may include one or more moieties which are selectively transported into specific cells or organs, thus enhance targeted drug delivery (see, e.g., V. V. Ranade (1989) J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms can be dictated by and directly dependent on (a) the unique characteristics of the active compound and the particular therapeutic effect to be achieved, and (b) the limitations inherent in compounding such an active compound for the treatment of sensitivity in individuals.

An exemplary, non-limiting range for a therapeutically or prophylactically effective amount of a composition described herein is 0.1-20 mg/kg, more preferably 1-10 mg/kg. The composition can be administered by intravenous infusion at a rate of less than 30, 20, 10, 5, or 1 mg/min to reach a dose of about 1 to 100 mg/m² or about 5 to 30 mg/m². It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit.

A pharmaceutical composition may include a “therapeutically effective amount” or a “prophylactically effective amount” of a zinc finger protein or a nucleic acid encoding it, e.g., a protein or nucleic acid described herein. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein to elicit a desired response in the individual. A therapeutically effective amount is also one in which any toxic or detrimental effects of the composition are outweighed by the therapeutically beneficial effects. A “therapeutically effective dosage” preferably inhibits a measurable parameter, e.g., inflammation or tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention are kits including the zinc finger protein or a nucleic acid that encodes it and instructions for use, e.g., treatment, prophylactic, or diagnostic use. In an embodiment in which the zinc finger protein regulates the VEGF-A gene, the instructions for therapeutic applications include suggested dosages and/or modes of administration in a patient with a cancer or neoplastic disorder, or angiogenesis related disorder (e.g., certain inflammatory disorders). The kit can further contain a least one additional reagent, such as a diagnostic or therapeutic agent, e.g., a diagnostic or therapeutic agent as described herein, and/or one or more additional zinc finger proteins or nucleic acids, formulated as appropriate, in one or more separate pharmaceutical preparations.

Treatments

Zinc finger proteins that can regulate an endogenous gene, particularly proteins that can regulate the VEGF-A gene, have therapeutic and prophylactic utilities. For example, these proteins or nucleic acid encoding them can be administered to cells in culture, e.g. in vitro or ex vivo, or in a subject, e.g., in vivo, to treat, prevent, and/or diagnose a variety of disorders, such as cancers, particularly metastatic cancers, an inflammatory disorder, and other disorders associated with increased angiogenesis.

As used herein, the term “treat” or “treatment” is defined as the application or administration of an agent which enables a zinc finger protein to enter a cell and regulate gene expression, to a subject, e.g., a patient, or application or administration of the agent to an isolated tissue or cell, e.g., cell line, from a subject, e.g., a patient, who has a disorder (e.g., a disorder as described herein), a symptom of a disorder or a predisposition toward a disorder, with the purpose to cure, heal, alleviate, relieve, alter, remedy, ameliorate, improve or affect the disorder, the symptoms of the disorder or the predisposition toward the disorder.

In one embodiment, “treating a cell” or “treating a tissue” refers to a reduction in at least one activity of a cell, e.g., VEGF-A production, angiogenesis stimulation, proliferation, or other activity of a cell, e.g., a hyperproliferative cell or cell in or near a tissue, e.g., a tumor. Such reduction can include a reduction, e.g., a statistically significant reduction, in the activity of a cell or tissue (e.g., metastatic tissue) or the number of the cell or size of the tissue, the amount or degree of blood supply to the tissue. An example of a reduction in activity is a reduction in migration of the cell (e.g., migration through an extracellular matrix), a reduction in blood vessel formatin, or a reduction in cell differentiation. Another example is an activity that, directly or indirectly, reduces inflammation or an indicator of inflammation.

As used herein, an amount of a zinc finger protein or a nucleic acid encoding it effective to treat a disorder, or a “therapeutically effective amount” refers to an amount of the protein or nucleic acid which is effective, upon single or multiple dose administration to a subject, in treating a cell.

As used herein, an amount of an zinc finger protein or a nucleic acid encoding it effective to prevent a disorder, or a “a prophylactically effective amount” of the protein or nucleic acid refers to an amount of the protein or the nucleic acid encoding it, which is effective, upon single- or multiple-dose administration to the subject, in preventing or delaying the occurrence of the onset or recurrence of a disorder, e.g., a cancer, angiogenesis-based disorder, or inflammatory disorder.

As used herein, the term “subject” is intended to include human and non-human animals. Exemplary subjects include a human patient having a disorder characterized by abnormal cell proliferation or cell differentiation. The term “non-human animals” includes all non-human vertebrates, e.g., non-mammals (such as chickens, amphibians, reptiles) and mammals, such as non-human primates, sheep, dog, cow, pig, etc.

In one embodiment, the subject is a human subject. In one embodiment, the composition of a zinc finger protein or a nucleic acid encoding it can be administered to a non-human mammal (e.g., a primate, pig or mouse) for veterinary purposes or as an animal model of human disease. Regarding the latter, such animal models may be useful for evaluating the therapeutic efficacy of the composition (e.g., testing of dosages and time courses of administration).

In one embodiment, the invention provides a method of treating a neoplastic disorder. The method can include the steps of contacting a cell of a subject with an zinc finger protein or a nucleic acid encoding it, e.g., a zinc finger protein that regulates VEGF-A or a nucleic acid encoding it, e.g., as described herein, in an amount sufficient to treat or prevent the neoplastic disorder. For example, the disorder can be caused by a cancerous cell, a tumor cell or a metastatic cell. The subject method can be used on cells in culture, e.g. in vitro or ex vivo. For example, cancerous or metastatic cells (e.g., renal, urothelial, colon, rectal, lung, breast, endometrial, ovarian, prostatic, or liver cancerous or metastatic cells) can be cultured in vitro in culture medium and the contacting step can be effected by adding the zinc finger protein or a nucleic acid encoding it to the culture medium. The method can be performed on cells (e.g., cancerous or metastatic cells) present in a subject (e.g., a human subject), as part of an in vivo (e.g., therapeutic or prophylactic) protocol. For in vivo embodiments, the contacting step is effected in a subject and includes administering the zinc finger protein or a nucleic acid encoding it to the subject under conditions effective to permit regulation of the VEGF-A gene in cells of the subject.

The method can be used to treat a cancer. As used herein, the terms “cancer”, “hyperproliferative”, “malignant”, and “neoplastic” are used interchangeably, and refer to those cells an abnormal state or condition characterized by rapid proliferation or neoplasm. The terms include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be benign, premalignant or malignant.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth. Metastatic lesions of the aforementioned cancers also can be treated or prevented using a method or composition described herein.

The subject method can be useful in treating malignancies of the various organ systems, such as those affecting lung, breast, lymphoid, gastrointestinal (e.g., colon), and genitourinary tract, prostate, ovary, pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include choriocarcinomas and those forming from tissue of the cervix, lung, prostate, breast, endometrium, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is recognized by those skilled in the art and refers to malignant tumors of mesenchymal derivation.

The subject method also can be used to inhibit the proliferation of hyperplastic/neoplastic cells of hematopoietic origin shown to express VEGF-A.

Methods of administering zinc finger proteins or nucleic acids are described in “Pharmaceutical Compositions”. Suitable dosages of the molecules used will depend on the age and weight of the subject and the particular drug used.

A zinc finger protein or a nucleic acid encoding it can be coupled to label, e.g., for imaging in a subject after it is delivered to a subject. Suitable labels include MRI-detectable labels or radiolabels.

A zinc finger protein or a nucleic acid encoding it described herein can be administered alone or in combination with one or more of the existing modalities for treating cancers, including, but not limited to: surgery; radiation therapy, and chemotherapy.

A zinc finger protein or a nucleic acid encoding it, particularly one that can regulate (e.g., reducing expression of) the VEGF-A gene, can be administered alone or in combination with one or more of the existing modalities for treating an inflammatory disease or disorder. Exemplary inflammatory diseases or disorders include: acute and chronic immune and autoimmune pathologies, such as, but not limited to, rheumatoid arthritis (RA), juvenile chronic arthritis (JCA), psoriasis, graft versus host disease (GVHD), scleroderma, diabetes mellitus, allergy; asthma, acute or chronic immune disease associated with an allogenic transplantation, such as, but not limited to, renal transplantation, cardiac transplantation, bone marrow transplantation, liver transplantation, pancreatic transplantation, small intestine transplantation, lung transplantation and skin transplantation; chronic inflammatory pathologies such as, but not limited to, sarcoidosis, chronic inflammatory bowel disease, ulcerative colitis, and Crohn's pathology or disease; vascular inflammatory pathologies, such as, but not limited to, disseminated intravascular coagulation, atherosclerosis, Kawasaki's pathology and vasculitis syndromes, such as, but not limited to, polyarteritis nodosa, Wegener's granulomatosis, Henoch-Schonlein purpura, giant cell arthritis and microscopic vasculitis of the kidneys; chronic active hepatitis; Sjogren's syndrome; psoriatic arthritis; enteropathic arthritis; reactive arthritis and arthritis associated with inflammatory bowel disease; and uveitis.

Inflammatory bowel diseases (IBD) include generally chronic, relapsing intestinal inflammation. IBD refers to two distinct disorders, Crohn's disease and ulcerative colitis (UC). The clinical symptoms of IBD include intermittent rectal bleeding, crampy abdominal pain, weight loss and diarrhea. A clinical index can also be used to monitor IBD such as the Clinical Activity Index for Ulcerative Colitis. See also, e.g., Walmsley et al. Gut. 1998 July; 43(1):29-32 and Jowett et al. (2003) Scand J Gastroenterol. 38(2):164-71.

A zinc finger protein or a nucleic acid encoding it can be used to treat or prevent one of the foregoing diseases or disorders. For example, the protein can be administered (locally or systemically) in an amount effective to ameliorate at least one symptom of the respective disease or disorder. The protein may also ameliorate inflammation, e.g., an indicator of inflammation, e.g., such as local temperature, swelling (e.g., as measured), redness, local or systemic white blood cell count, presence or absence of neutrophils, cytokine levels, and so forth. It is possible to evaluate a subject, e.g., prior, during, or after administration of the protein, for one or more of indicators of inflammation, e.g., an aforementioned indicator.

A zinc finger protein or a nucleic acid encoding it, particularly one that can regulate (e.g., increase expression of) the VEGF-A gene, can be administered alone or in combination with one or more of the existing modalities for treating a wound, e.g., to promote wound healing. For example, generally, activation of VEGF-A can increase formation of new blood vessels and capillaries. The protein or nucleic acid can also be used for ameliorating surgery, burn, traumas, ulcers, bone fractures, and other disorders that require increased angiogenesis.

A zinc finger protein or a nucleic acid encoding it, particularly one that can regulate (e.g., increase expression of) the VEGF-A gene, can be administered alone or in combination with one or more of the existing modalities for treating a cardiovascular disorder, e.g., e.g., ischemic heart disease, peripheral artery disease, or coronary artery disease.

The present invention will be described in more detail through the following practical examples. However, it should be noted that these examples are not intended to limit the scope of the present invention.

### EXAMPLE 1

### Gel Shift Assays

This example provides a method of evaluating the DNA binding properties of zinc finger proteins in vitro. Zinc finger proteins were expressed in E. coli, purified, and used in gel shift assays. The DNA segments encoding zinc finger proteins were inserted into pGEX-4T2 (Pharmacia Biotech). These constructs were expressed in E. coli strain BL21 to produce fusion proteins that include the zinc finger proteins connected to GST (Glutathione-S-transferase). The fusion proteins were purified using glutathione affinity chromatography (Pharmacia Biotech, Piscataway, N.J.) and then digested with thrombin. Thrombin cleaves the linker sequence between the GST moiety and zinc finger proteins.

Various amounts of a zinc finger protein were incubated with a radioactively labeled probe DNA for one hour at room temperature in 20 mM Tris pH 7.7, 120 mM NaCl, 5 mM MgCl₂, 20 μM ZnSO₄, 10% glycerol, 0.1% Nonidet P-40, 5 mM DTT, and 0.10 mg/mL BSA (bovine serum albumin), and then the reaction mixtures were subjected to gel electrophoresis. Distribution of the probe in the gel was quantitated by PHOSPHORIMAGER™ analysis (Molecular Dynamics). Dissociation constants (K_{d}) were determined as described (Rebar and Pabo (1994) Science 263:671-673).

We have previously found that zinc finger proteins that function in an in vivo yeast assay also have biochemical activity. In general, when a zinc finger protein, e.g., having three zinc finger domains, binds a DNA sequence with a dissociation constant lower than 1 nM, it allows cell growth in the one-hybrid yeast cell assay described in US 2002-0061512, whereas when a zinc finger protein binds a DNA sequence with a dissociation constant higher than 1 nM, it does not allow cell growth in that assay. Zinc finger proteins that bind with a dissociation constant of greater than 1 nM but less than 50 nM can also be useful. For example, additional fingers can be added to those zinc fingers to produce tighter or more specific binders.

The in vitro assay can also be adapted to evaluate binding by an individual zinc finger domain to a particular three or four basepair site. In one implementation, the individual zinc finger domain is evaluated in the context of fingers 1 and 2 of Zif268 and a target site that includes (i) basepairs recognized by fingers 1 and 2 and (ii) the particular three or four basepair site.

### EXAMPLE 2

### Construction of Individual Three-Fingered Proteins

This example provides a method for constructing a nucleic acid encoding a chimeric three-fingered protein. The vector P3 (Toolgen, Inc.) was used to express chimeric zinc finger proteins in mammalian cells. P3 was constructed by modification of the pcDNA3 vector (Invitrogen, San Diego Calif.). A synthetic oligonucleotide duplex having compatible overhangs was ligated into the pcDNA3 vector digested with HindIII and XhoI. The duplex contains nucleic acid that encodes the hemagglutinin (HA) tag and a nuclear localization signal. The duplex also includes BamHI, EcoRI and NotI and BglII restriction site sites and a stop codon. Further, the XmaI site in SV40 origin of the resulting vector was destroyed by digestion with XmaI, filling in the overhanging ends of the digested restriction site, and religation of the ends.

The following is one exemplary method for constructing a plasmid that encodes a chimeric zinc finger protein with multiple zinc finger domains. First, an insert that encodes a single zinc finger domain was inserted into a vector (the P3 vector) that harbored a sequence encoding a single zinc finger domain. The result of this cloning is a plasmid that encodes a zinc finger protein with two zinc finger domains. A zinc finger domain insert consisting of two zinc finger domains was prepared by the above method and cloned into AgeI/NotI-linearized vector P3 having one or two zinc finger domains to obtain a plasmid containing a zinc finger protein gene consisting of three or four zinc finger domains.

Genes encoding chimeric zinc finger proteins were then cloned into pre-prepared plasmids that encode a functional domain, e.g., p65 transcriptional activation domain, a Kid transcriptional repression domain, or a KOX transcriptional repression domain. The plasmids that include the genes encoding chimeric zinc finger proteins were digested with EcoRI/NotI and ligated into plasmids linearized with the same enzymes. The cloning site in the acceptor plasmids (pLFD-p65, pLFD-KRAB, pLFD-KOX) placed the sequence encoding the zinc finger domains in a position that results in the DNA binding region being N-terminal to the functional domain. The resulting constructs encode a protein that includes, from N- to C-terminus: HA-tag, Nuclear localization signal, zinc finger protein and the functional domain.

### EXAMPLE 3

### In vivo Assays for Three-Fingered Proteins with Human Zinc Finger Domains

An in vivo repression assay was used to determine if the new three-fingered proteins were functional in vivo. See, for example, Kim and Pabo ((1997) J Biol Chem 272:29795-29800). The assay utilized a luciferase reporter construct in which a target site is located at a position comparable to the position of the Zif268 site in the construct of Kim and Pabo, supra.

The luciferase reporter plasmids were constructed from pAS-modi, a modified version of pGL3-TATA/Inr (Kim and Pabo, supra). These reporters utilize firefly luciferase as the reporter protein. The SacI site upstream of the TATA box was deleted from pAS-modi. A new SacI site was inserted following the transcription initiation site. Different reporter plasmids were made for each of the different zinc finger proteins. To construct each plasmid, an oligomer containing a given nine basepair binding site that is predicts to interact with a particular zinc finger protein was inserted into the plasmid. The plasmid pAS-modi was digested with SacI and HindIII, and the oligomer was inserted. This manipulation replaces 14 base pairs at a position 12 basepairs downstream from the transcription initiation site. The resulting reporter plasmids were named p1G-ZFP ID, wherein ID was the name of the corresponding zinc finger protein.

The in vivo activity assay for a particular three-fingered protein was carried out as follows. HEK 293 cells were transfected with four plasmids: 14 ng of a plasmid expressing the particular three-fingered protein; 14 ng of the reporter plasmid described above; 70 ng of a plasmid that expresses GAL4VP16; and 1.4 ng of a plasmid that expresses Renilla luciferase. The GAL4-VP16 activates transcription of the minimal synthetic promoter in the reporter absent repression by a particular three-fingered protein. The ability of different zinc finger proteins was compared to other three-fingered proteins. The plasmid expressing Renillar luciferase controlled for transfection efficiency.

LIPOFECTAMINE™ (Gibco-BRL) was used for the transfection procedures. Cells were transfected at 30-50% confluency in wells of a 96 well plate. The cells were incubated for two days prior to harvesting for the luciferase assay. Then luciferase activities were measured using the DUAL-LUCIFERASE™ Reporter Assay System (Promega). The observed firefly luciferase activity was normalized using the observed level of Renilla luciferase. The extent of repression or “fold-repression” was calculated by dividing a value for normalized reporter expression in the absence of a zinc finger protein by a value for normalized reporter expression in the presence of the zinc finger protein.

Zinc finger proteins were classified as satisfying a high stringency cut-off value if they repressed transcription at least 2-fold in the transfection assay or as satisfying a low stringency cut-off value if they repressed between 1.5 and 2-fold in the transfection assay.

### EXAMPLE 4

### Binding Assay Result of ZFPs with Their Specific Reporter

Gel shift assays were used to correlate activity observed in the in vivo assays to binding affinity. The binding of Zif268 to different target sequences was evaluated using gel shift assays and the transfection assay described above in Example 3. A good correlation was observed between the dissociation constants measured by gel shift assays and the level of transcriptional repression in the transfection assays described above. In general, zinc finger proteins exhibiting more than 2-fold repression (that is, 50% repression) in the transfection assays showed a dissociation constant of less than 1 nM as determined by gel shift assays.

### EXAMPLE 5

### Characterization of Three-Fingered Proteins

Two types of “three-finger” chimeric zinc finger proteins were constructed. One type includes chimeric proteins that are composed exclusively of wild-type human zinc finger domains, i.e., domains that are identical to naturally-occurring human zinc finger domains. The other type includes chimeric proteins that include zinc finger domains that are not identical to a naturally-occurring zinc finger domain. The latter zinc finger domains were typically identified by in vitro mutagenesis of a naturally-occurring zinc finger domain followed by phage display selection. Such mutant domains have avoided the scrutiny of natural evolution.

A total of 36 zinc finger domains, 18 human zinc finger domains and 18 mutated zinc finger domains, were used to assemble a set of test three-fingered proteins. The mutated zinc finger domains have been reported in Choo and Klug (1994) Proc. Natl. Acad. Sci. USA 91:11168-11172; Desjarlais and Berg (1994) Proc. Natl. Acad. Sci. USA. 91:11099-11103; Dreier et al. (2001) J. Biol. Chem. 276:29466-29478; Dreier et al. (2000) J. Mol. Biol. 303:489-502; Fairall et al. (1993) Nature 366:483-487; Greisman and Pabo (1997) Science. 275:657-661; Kim and Pabo (1997) J. Biol. Chem. 272:29795-29800; and Segal et al. (1999) Proc. Natl. Acad. Sci. USA 96:2758-2763. See also Table 9 of US 2003-165997. Nucleic acids encoding the 36 domains were individually subcloned into P3 vector digested with EcoRI and NotI, and the resulting plasmids were used as starting material for the chimeric zinc finger protein construction.

Nucleic acids encoding chimeric three-fingered proteins were prepared by two different methods. In the first method, nucleic acids encoding all the zinc finger domains were randomly mixed, and three-fingered constructs were randomly picked for further analysis. Each construct was sequenced to determine the component zinc finger domains in the polypeptide that it encodes. Subsequently, target DNA sequences were synthesized for each randomly assorted three-fingered protein. Target DNA sequences were based on the expected preferred target site. The targets were cloned into the luciferase reporter vector described above. This approach is referred to as “zinc finger protein-first” approach.

In the second method, nucleic acid encoding chimeric three-fingered proteins were assembled based on a given target DNA sequences. A computer algorithm was used to match recognition sites of zinc finger domains and target DNA sequences. Promoter sequences of known genes were used as the input target DNA sequences. The promoter sequences were scanned to identify segments that are nine nucleotides in length and that are acceptable target sites for recognition by chimeric three-fingered proteins given the available collection of zinc finger domains. Once identified, a nucleic acid was constructed that encoded the chimeric three-fingered proteins. This approach is referred to as “target site-first” approach.

Zinc finger domains that include an aspartate residue at position 2 of the base contacting residues were analyzed with special consideration. Such zinc finger domains include RDER1, RDHT, RDNR, RDKR, RDTN, TDKR, and NDTR. The X-ray co-crystal structure of Zif268 bound to DNA showed that an aspartate at position 2 can form a hydrogen bond with a base outside of the 3-basepair subsite recognized by zinc fingers. As a result, the RDER finger containing an aspartate residue at position 2 prefers the 4-basepair site: 5′-GCG (G/T)-3′. The computer algorithm accounted for this additional specificity. Randomly-assembled three-fingered proteins that include a finger having aspartate at position 2 and that violate this rule for the 4-bp site were excluded in other analyses described herein.

A total of 153 three-fingered proteins were constructed from both the “zinc finger protein-first” and the “target site-first” approaches. These proteins were tested using the transient cotransfection assay described in Example 3.

31 of 153 chimeric zinc finger proteins demonstrated greater than 2-fold repression, the high stringency criterion (RF≧2; RF=fold repression). Of the proteins constructed entirely from naturally-occurring human zinc finger domains, 28.1% (27 of 96) exceeded the high stringency criterion and 59.4% exceeded the low stringency criterion (RF≧1.5). Of the proteins constructed from two naturally-occurring zinc finger domains and one mutated domain, 33.3% exceeded the high stringency criterion, and only 20% exceeded the low stringency criterion.

In contrast, of the 17 proteins constructed from one human domain and two mutated domains, only one protein (5.9%) exceeded the high stringency criterion, and only two proteins (11.8%) exceeded the low stringency criterion. Strikingly, no zinc finger proteins composed exclusively of mutated domains satisfied the high stringency criterion in the repression assay. Only one such protein (4%) satisfied the low stringency criterion. These results indicate that naturally-occurring human zinc finger domains are frequently better building blocks for the construction of new DNA-binding proteins than mutated domains.

### EXAMPLE 6

### Designed Chimeric Zinc Finger Proteins that Bind to the VEGF-A Gene

In this example, we designed chimeric zinc finger proteins that bind to DNA elements in the human vascular endothelial growth factor A (VEGF-A) gene. The −950 to +450 region of the VEGF-A promoter was scanned to identify nine nucleotide sites that are compatible for recognition by available combinations of zinc finger domains in a three-fingered configuration.

We constructed several DNA constructs encoding zinc finger proteins that include domains designed to recognize such nine nucleotide sites. The proteins were expressed in E. coli and purified. We evaluated their DNA binding specificity using a SELEX (Systematic Evolution of Ligands by EXponential enrichment) experiment. Many zinc finger proteins that were designed to target the VEGF-A promoter demonstrated the expected DNA-binding specificities. Nearly all of the consensus sequences obtained from the SELEX analyses were identical to the intended target sequences in the VEGF-A gene. One exemplary zinc finger protein, termed F121, showed a consensus sequence that differed from the intended target sequence by one base at a position where the corresponding zinc finger shows degeneracy in base recognition.

Transcription factors that include a DNA binding domain with these artificial zinc fingers were generated by fusing nucleic acids encoding the three zinc finger domains to a nucleic acid encoding either the p65 or VP16 activation domain. The resulting nucleic acid was inserted into an expression plasmid.

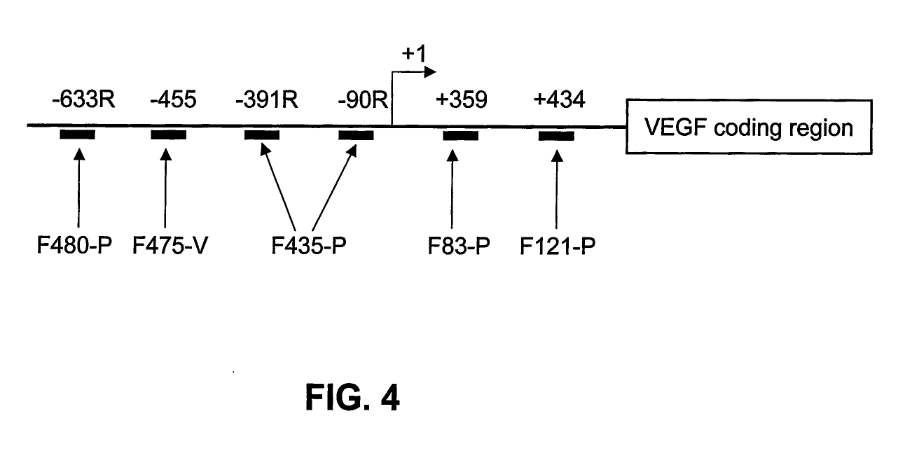

FIG. 4 shows the locations of the DNA binding sites in the VEGF-A promoter that are recognized by these chimeric zinc finger proteins. The human VEGF-A promoter contains at least two DNase I-hypersensitive regions. The binding of engineered zinc finger proteins transcription factors to these sites can activate VEGF-A gene expression. F480 was designed to recognize a site at about −633R (“R” designates the reverse strand). F475 was designed to recognize a site at about −455. F435 was designed to recognize a site at about −391R and a site at about −90R. F83 was designed to recognize a site at about +359. F121 was designed to recognize a site at about +434.

We found that regardless of the location of the binding sites, four zinc finger proteins (F480, F475, F121, and F435) that we tested activated not only a luciferase reporter gene under the control of the VEGF-A promoter, but also the endogenous VEGF-A gene itself. An ELISA on media from the transiently transfected cells indicated that these chimeric zinc finger proteins also up-regulated production of the VEGF-A protein 13- to 21-fold.

When two of the chimeric zinc finger proteins, F435 and F121, were fused individually to the KRAB repression domain, they each actively repressed VEGF-A expression in HEK 293 cell. Control cells that had been transfected with the parental expression vector (which contained no zinc finger protein coding sequences) did not show any increase or decrease in VEGF-A mRNA or protein levels.

The protein F83 did not show any effect on the levels of VEGF-A mRNA or protein in these assays). This may be due to the binding of some other protein to the target site or to the local chromatin structure, which might have rendered the target DNA inaccessible to the zinc finger protein. There was no absolute correlation between the levels of VEGF-A expression by these zinc finger proteins and their DNA-binding affinities or their expression levels in cells.

To investigate the specificity of zinc finger proteins on a genome-wide scale, we performed DNA microarray experiments with 293 cell lines that had been stably transfected with DNA constructs that encode one of the following three zinc finger transcription factors: F121-p65, F435-p65, and F475-VP16. Fifty-one of 7458 genes were regulated by all three zinc finger transcriptional activator proteins. Forty-nine were up-regulated more than two-fold, and two were down-regulated more than two-fold. Most of these co-regulated genes appear to be closely associated with VEGF-A function. Many of them are regulated by VEGF-A, involved in angiogenesis or hypoxia, or expressed in vascular endothelial cells. Therefore, it is likely that these genes are downstream targets of VEGF-A expression. In addition, numerous other genes were regulated by one or two of the zinc finger protein activators but not by all three tested proteins. Since these zinc finger proteins recognize nine basepairs site, it is possible that these zinc finger proteins directly regulate genes other than VEGF-A, e.g., by binding to identical or related target sites in other genes. Construction of four, five, or six-fingered proteins may improve specificity. Taken together, these data indicate that the described zinc finger proteins, which were assembled by shuffling naturally-occurring zinc finger domains, function in cells as transcriptional regulators of specific endogenous genes.

For example, a protein described herein may regulate one or more of the following genes: jun B proto-oncogene (N94468), EphA2 (H84481), EphB4 (AI261660), fibroblast growth factor receptor 3 (achondroplasia, thanatophoric dwarfism) (AA419620), FK506-binding protein 8 (388 kD) (N95418), protein kinase C, zeta (AA458993), v-erb-b2 avian erythroblastic leukemia viral oncogene homolog 3 (AA664212), lectin, galactoside-binding, soluble, 1 (galectin 1) (AI927284), protein phosphatase 2, regulatory subunit B (B56), alpha isoform (R59165), insulin-like growth factor 2 (somatomedin A) (N54596), plectin 1, intermediate filament binding protein, 500 kD (AA448400), Periplakin (AI703487), choline kinase (H09959), collagen, type VI, alpha 1 (H99676), adaptor-related protein complex 1, sigma 1 subunit (W44558), arrestin, beta 2 (AW009594), GATA-binding protein 2 (H00625), cyclin-dependent kinase inhibitor 1A (p21, Cip1) (AI952615), mitogen-activated protein kinase kinase kinase 11 (R80779), acetylcholinesterase (YT blood group) (AI360141), brain-specific Na-dependent inorganic phosphate cotransporter (AA702627), cellular retinoic acid-binding protein 1 (AA454702), cellular retinoic acid-binding protein 2 (AA598508), cadherin 13, H-cadherin (heart) (R41787), calcium channel, voltage-dependent, beta 3 subunit (R36947), carbonic anhydrase XI (N52089), troponin T1, skeletal, slow (AA868929), gamma-aminobutyric acid (GABA) B receptor, 1 (N70841), adenylate cyclase activating polypeptide 1 (pituitary) receptor type I (H09078), solute carrier family 4, anion exchanger, member 2 (erythrocyte membrane protein band 3-like 1) (W45518), glypican 1 (AA455896), protein C inhibitor (plasminogen activator inhibitor III) (W86431), cyclin-dependent kinase inhibitor 1C (p57, Kip2) (AI828088), zinc finger protein 43 (HTF6) (AA773894), zinc finger protein homologous to Zfp-36 in mouse (R38383), Meis (mouse) homolog 3 (AA703449), SWI/SNF related, matrix associated, actin dependent regulator of chromatin, subfamily d, member 3 (AA053810), ( ), unknown (R11526), unknown (AA045731), unknown (T51849), unknown (T50498), putative gene product (H09111), B/K protein (H23265), damage-specific DNA binding protein 2 (48 kD) (AA410404), dihydropyrimidinase-like 4 (AA757754), N-methylpurine-DNA glycosylase (N26769), protein tyrosine phosphatase, receptor type, N (R45941), fasciculation and elongation protein zeta 1 (zygin I) (H20759), lanosterol synthase (2,3-oxidosqualene-lanosterol cyclase) (AA437389), ( ), ( ), spennidine/spermine N1-acetyltransferase (AA011215), and a disintegrin-like and metalloprotease (reprolysin type) with thrombospondin type 1 motif, 1 (T41173). The expression of these protein or the genes that encode them can be regulated at least 0.5, 1.0, 2, 5, 10, or 50-fold, e.g., between 2 and 80-fold.

Exemplary sites in the VEGF promoter and proteins that can recognize them include:

TABLE 7
VEGF-A Promoter Sites (A)

| | | |
|---|---|---|
| Protein | Site | Sequence |

| | | |
|---|---|---|
| F475 | −455 | GAG CGG GGA |
| F121 | +434 | TGG GGG TGA |
| F435 | −90R | GGG CGG GGA |
| F547 | −665 | AAT AGG GGG |
| F2825 | +434 | TGG GGG TGA |

TABLE 8
VEGF-A Promoter Sites (B)

| | | |
|---|---|---|
| Protein | Site | Sequence |

| | | |
|---|---|---|
| F480 | −633R | GGG TGG GGG |
| F435 | −391R | GGG TGG GGA |
| F2828 | +435 | GGG GGT GAC |
| F625 | +435 | GGG GGT GAC |
| F2830 | +435 | GGG GGT GAC |
| F2838 | +435 | GGG GGT GAC |

TABLE 9
VEGF-A Promoter Sites (C)

| | | | SEQ |
|---|---|---|---|
| | | | ID |
| Protein | Site | Sequence | NO: |
| F2604 | −680 | GTT TGG GAG GTC | 76 |
| F2605 | −677 | TGG GAG GTC AGA | 77 |
| F2607 | −671 | GTC AGA AAT AGG | 78 |
| F2615 | −606 | GCC AGA GCC GGG | 79 |
| F2633 | −455 | GAG CGG GGA GAA | 80 |
| F2634 | −395R | GGG GAG AGG GAC | 81 |
| F2636 | −393R | GTG GGG AGA GGG | 82 |
| F2644 | −358R | GGG GCA GGG GAA | 83 |
| F2646 | −314R | GAC AGG GCC TGA | 84 |
| F2650 | −206 | GGT GGG GGT CGA | 85 |
| F2679 | +244R | CAA GTG GGG AAT | 86 |

TABLE 10
VEGF-A Promoter Sites (D)

| | | | SEQ |
|---|---|---|---|
| | | | ID |
| Protein | Site | Sequence | NO: |
| F2610 | −633R | GGG TGG GGG GAG | 87 |
| F2612 | −630R | AGG GGG TGG GGG | 88 |
| F2638 | −391R | GGG TGG GGA GAG | 89 |

TABLE 11
VEGF-A Promoter Sites (E)

| | | | |
|---|---|---|---|
| | | | SEQ |
| | | | ID |
| Protein | Site | Sequence | NO: |

| | | | |
|---|---|---|---|
| F109 | 536B | GAG CGA GCA GCG | 90 |
| F2608 | −668 | AGA AAT AGG GGG | 91 |
| F2611 | −631R | GGG GGT GGG GGG | 92 |
| F2617 | −603 | AGA GCC GGG GTG | 93 |
| F2619 | −554 | AGG GAA GCT GGG | 94 |
| F2623 | −495 | GTG GGT GAG TGA | 95 |
| F2625 | −475 | GTG TGG GGT TGA | 96 |
| F2628 | −468 | GTT GAG GGT GTT | 97 |
| F2629 | −465 | GAG GGT GTT GGA | 98 |
| F2630 | −462 | GGT GTT GGA GCG | 99 |
| F2634 | −395R | GGG GAG AGG GAC | 100 |
| F2635 | −394R | TGG GGA GAG GGA | 101 |
| F2637 | −392R | GGT GGG GAG AGG | 102 |
| F2642 | −385R | AGG GAC GGG TGG | 103 |
| F2643 | −382R | GAC AGG GAC GGG | 104 |
| F2648 | −282R | GAG GAG GGA GCA | 105 |
| F2651 | −203 | GGG GGT CGA GCT | 106 |
| F2653 | −184R | GAA GGG GAA GCT | 107 |
| F2654 | −181R | AAT GAA GGG GAA | 108 |
| F2662 | −124R | GCG GCT CGG GCC | 109 |
| F2667 | −85 | GGG CGG GCC GGG | 110 |
| F2668 | −30R | AAA AAA GGG GGG | 111 |
| F2673 | +77 | GCA GCG GTT AGG | 112 |
| F2682 | +283R | GGG GAA GTA GAG | 113 |
| F2689 | +342 | AGA GAA GTC GAG | 114 |
| F2697 | +357 | GAG AGA GAC GGG | 115 |
| F2699 | +366 | GGG GTC AGA GAG | 116 |
| F2703 | −632R | GGG GTG GGG GGA | 117 |
| F2702 | +474R | CAA GGG GGA GGG | 118 |

Construction of a Yeast Expression Plasmid for a Zinc Finger Library

We constructed an expression plasmid encoding a zinc finger transcription factor by modification of pPC86 (Chevray and Nathans (1992) Proc. Natl. Acad. Sci. USA 89:5789-5793). A gene encoding the Zif268 zinc finger protein was inserted between the SalI and EcoRI sites of pPC86 to generate pPCFM-Zif, in which the Gal4 activation domain is fused to the Zif268 domain. pPCFM-Zif was used as a vector for constructing libraries of zinc fingers. To construct human zinc finger libraries, DNA segments encoding zinc fingers were amplified from human genomic DNA using the polymerase chain reaction (PCR) (Promega, Madison, Wis.) and mixtures of degenerate PCR primers with the sequence His-Thr-Gly-Glu/Gln-Lys/Arg-Pro-Tyr/Phe, which is frequently found at the junction between zinc fingers in naturally-occurring zinc finger proteins. The 100-bp PCR products encoding the zinc fingers were digested with SacII and AvaI and inserted into pPCFM-Zif, which encodes hybrid transcription factors consisting of finger 1 and finger 2 of Zif268 and a zinc finger domain derived from the human genome (together forming three-fingered protein). The plasmid library was prepared from a total of 1.2×10⁶ E. coli transformants.

Reporter plasmids were prepared by inserting one of 64 pairs of complementary oligonucleotides that contained three copies of a 9-bp target sequence into pRS315(His) and pLacZi (Clontech, Palo Alto, Calif.).

Gap Repair Cloning of Human Zinc Finger Domains Selected from the Human Genome

Gap repair cloning of DNA sequences that encode individual zinc finger domains was carried out as described (Hudson et al. (1997) Genome Res. 7:1169-1173). To clone a DNA segment that encode a zinc finger, two overlapping oligonucleotides were synthesized. Each oligonucleotide included a 21-bp common tail at its 3′ end for a second round of PCR as well as a specific sequence that can anneal to the nucleic acid sequence that encodes the individual zinc finger domain. DNA sequences encoding zinc fingers were amplified from human genomic DNA with an equimolar mixture of two corresponding oligonucleotides.

Amplification products from the initial round of PCR were used as templates in a second round of PCR. The primers for the second round of PCR had two regions, one identical to a segment of pPCFM-Zif and another identical to the 21-bp common tail. A mixture of the second-round PCR products and linearized pPCFM-Zif that had been digested with MscI and EcoRI were transformed into the yW1(MATα Δgal4 Δgal80 lys2801 his3-Δ200 trp1-Δ63 leu2 ade2-101CYH2) yeast strain. A total of 823 human zinc fingers were cloned by this method. Many were used in our in vivo selection systems described herein.

In Vivo Selection of Zinc Finger Domains

Yeast mating was used to facilitate identification of zinc fingers that bind to each three basepair target site. The zinc finger library was introduced into the yW1 (MATα) strain, and ˜1.47×10⁶ independent transformed yeast colonies were generated. Aliquots of these transformed cells were mated for 5 h at 30° C. with the haploid yeast strain yW1a (MATa), which contained the 64 reporter plasmids in each of two sets (one for each of the reporter genes). The reporter plasmids contained three copies of the target DNA sequences adjacent to the coding regions of either the LacZ or HIS3 genes. The resulting diploids were plated on selective media that contained X-gal (40 μg/ml) and 3-amino triazole (3-AT) (1 mM) but lacked histidine. Plasmids isolated from blue (positive) colonies were re-transformed to confirm the results and sequenced to identify their encoding zinc finger domains. The binding affinity and specificity of each zinc finger fused to fingers 1 and 2 of Zif268 were determined both in yeast and by EMSA. These methods are described below.

Construction of Three-Fingered Proteins Using Selected Zinc Fingers as Modular Building Blocks

A modified version of the pcDNA3 (Invitrogen, Carlsbad, Calif.) vector (P3) was used as a parental vector for expressing zinc finger proteins in mammalian cells. P3 contains an HA tag and a nuclear localization signal, both of which were inserted 3′ to the initiation codon. DNA segments that encode individual zinc finger domains were subcloned into the P3 vector between the EcoRI and NotI sites, and the resulting plasmids were used as starting material for chimeric zinc finger protein construction. New three-fingered proteins were prepared by two different methods. In the first method, all the zinc fingers were mixed, and assembled three-fingered constructs were randomly chosen for further analysis. In the second method, new three-fingered proteins were designed to target specific DNA sequences. To this end, we used a simple computer algorithm that finds a match between recognition sites of zinc fingers and target DNA sequences. We used promoter sequences of known genes as the input DNA sequences and identified three-fingered proteins that should bind to nine basepair DNA elements within the input sequences.

Zinc finger proteins that target the VEGF-A gene were constructed by this method. The constructed zinc finger proteins were tested for their DNA binding ability and affinity in mammalian cells as described previously. Kim and Pabo (1997) J. Biol. Chem. 272, 29795-29800; Kim and Pabo (1998) Proc. Natl. Acad. Sci. USA 95, 2812-2817; and Kang and Kim (2000) J. Biol. Chem. 275:8742-8748. The reporter plasmid for the assay was constructed using pGL3-TATA/Inr which harbors the firefly luciferase gene as the reporter.

To connect functional domains to the zinc finger proteins, the transcriptional activation domain of p65 (amino acids 288-548) and VP16 (amino acids 413-490) were amplified by PCR using pairs of specific oligomers, and the PCR products for p65 and VP16 were cloned separately into P3 to generate pLFD-p65 and pLFD-VP16, respectively. Nucleic acids that encode zinc finger proteins that target the VEGF-A promoter were inserted into pLFD-p65 or VP16 to express zinc finger protein-activation domain (AD) fusions proteins (ZFP-AD). Real-time PCR, ELISA, and microarray analyses were carried out to determine whether these ZFP-ADs activate the VEGF-A gene. In addition, SELEX was performed to test whether these proteins recognize the appropriate target DNA sequences. See below.

Binding Affinity and Specificity of Human Zinc Finger Domains

Plasmids isolated from blue yeast colonies (see section entitled “In vivo selection of zinc finger domains”) were individually retransformed into yW1 cells. For each isolated plasmid, re-transformed yW1 cells were mated to yW1a cells that contained each of the 64 LacZ reporter plasmids. The resulting cells were then spread onto minimal media that contained X-gal and histidine but lacked tryptophan and uracil. Using the GEL-DOC™ system (Bio-Rad, Hercules, Calif.), we measured the intensity of the blue color for each colony to determine the DNA-binding affinities and specificities of each of the zinc finger domains that were fused to fingers 1 and 2 of Zif268. Control experiments with the Zif268 protein indicated that positive interactions between a zinc finger domain and a target binding site in the promoter of the LacZ reporter yielded dark to pale blue colonies (the blue intensity is proportional to the binding affinity) and that negative interactions yielded white colonies.

Electrophoretic Mobility Shift Assay (EMSA)

DNA segments that encode zinc finger proteins were isolated by digestion with SalI and NotI, and were inserted into pGEX-4T2 (Amersham Pharmacia, Uppsala, Sweden). Zinc finger proteins were expressed in E. coli strain BL21(DE3) as fusion proteins linked to glutathione-S-transferase (GST). The fusion proteins were purified using glutathione affinity chromatography (Amersham Pharmacia) and then digested with thrombin. This cleavage event severs the connection between the GST moiety and the zinc finger proteins. In this case, purified zinc finger proteins contained fingers 1 and 2 of Zif268 fused to selected zinc fingers in position 3 at the C-terminus. Probe DNAs were synthesized, annealed, labeled with ³²p using T4 polynucleotide kinase, and EMSAs were carried out as described. Kim and Pabo (1997) J. Biol. Chem. 272, 29795-29800 and Kim and Pabo (1998) Proc. Natl. Acad. Sci. USA 95, 2812-2817. The same procedure can be used to test other zinc finger proteins.

Transcriptional Regulation of Endogenous VEGF

Human embryonic kidney 293 cells were maintained in Dulbecco's modified Eagle medium (DMEM) supplemented with 100 units/ml penicillin, 100 μg/ml streptomycin, and 10% fetal bovine serum (FBS). For the luciferase assay, 10⁴ cells/well were pre-cultured in a 96-well plate. Using a LIPOFECTAMINE™ transfection kit (Life Technologies, Rockville, Md.), 293 cells were transfected with 25 ng of a reporter plasmid in which the native VEGF-A promoter was fused to the luciferase gene in pGL3-basic (Promega), and 25 ng of a plasmid encoding a zinc finger protein. After 48 h of incubation, luciferase activity was measured with a DUAL LUCIFERASE™ assay kit (Promega) using a TD-20/20 luminometer (Turner Designs Inc., Sunnyvale, Calif.).

For reverse transcriptase-PCR (RT-PCR) analyses and ELISA, 10⁵ cells/well were pre-cultured in 1 ml of culture medium (supplemented with 10% FBS but deprived of antibiotics) in a 12-well culture plate for 24 h at 37° C. in a humid atmosphere containing 5% CO₂. The cells were then transfected with DNA using a LIPOFECTAMINE™ transfection kit (Life Technologies). Briefly, 1 μg of a plasmid encoding a zinc finger protein was added to 5 μl plus reagent in a total of 50 μl DMEM, and this solution was then mixed with another 50 μl of DMEM containing 2 μl of LIPOFECTAMINE™ reagent. After 15 min of incubation, the entire 100 μl mixtures were added to cells in a culture plate, and the cells were grown for an additional 48 h. The cells and culture supernatants were harvested for RT-PCR analysis and ELISA.

Quantitative RT-PCR

Total cellular RNA was extracted from TRIZOL™-lysates according to the manufacturer's instructions (Life Technologies). The reverse transcription reactions were performed with 4 μg total RNA using oligo-dT as the first-strand synthesis primer for mRNA and the MMLV reverse transcriptase provided in the SUPERSCRIPT™ first-strand synthesis system (Life Technologies). To analyze mRNA quantities, 1 μl of the first-strand cDNAs generated from the RT reactions were amplified using VEGF-A-specific primers. The initial amounts of RNA were normalized to glyceraldehydes-3-phosphate dehydrogenase (GAPDH) mRNA concentrations that had been calculated by specific amplification using GAPDH-specific primers. The amplification of VEGF-A and GAPDH-specific cDNAs was monitored and analyzed in real-time with a QUANTITECT SYBR™ kit (QIAGEN, Valencia, Calif.) and ROTORGENE™ 2000 real-time cycler (Corbett, Sydney, Australia) and was quantified using serial dilution of the standards included in the reactions.

ELISA

The kidney 293 cell culture supernatants were briefly centrifuged for 5 min to remove cells and cell debris. VEGF protein that accumulated in the culture medium (100 ul each) and dilutions of a recombinant human VEGF-A protein standards were analyzed using sandwich ELISA (enzyme linked immunosorbent assay), wherein the supernatant of culture was reacted with a anti-human VEGF antibody (R&D systems; AF-293-NA), biotinylated anti-human VEGF antibody (R&D systems; BAF293), streptavidin alkaline phosphatase. The antigen-antibody complex was reacted with pNPP (p-Nitrophenyl phosphate) dissolved in pNPP buffer (Chemicon; ES011). VEGF-A concentrations in the samples were determined from the absorbance at 405 nm which was measured with POWERWAVE™ X340(Bio-TEK Instrument Inc., Winooski Vt.).

DNA Microarray Analysis of FlpTRex-293 Cell Lines Stably Expressing Zinc Finger Proteins

Plasmids encoding ZFPs designed to target the VEGF-A promoter were stably introduced into FlpTRex-293 cell lines (Invitrogen) essentially as described in the manufacturer's protocol. Briefly, the HindIII-XhoI fragment from a pLFD-p65 or a pLFD-VP16 vector that contained DNA segments encoding zinc finger proteins was subcloned into pCDNA5/FRT/TO (Invitrogen). The resulting plasmids were cotransfected with pOG44 (Invitrogen) into FlpTRex-293 cells, and stable integrants were screened. The resulting cell lines express ZFP-p65 or ZFP-VP16 upon doxycycline induction.

DNA microarrays containing 7458 human expressed sequence tag (EST) clones were provided by Genomic Tree, Inc. (Taejon, Korea). FlpTRex-293 cells stably expressing ZFP-p65 or ZFP-VP16 were grown with (+Dox) or without (−Dox) 1 μg/ml Doxycycline for 48 h. Total RNA was prepared from each sample. RNA from a -Dox sample was used as the reference (Cy3). Microarray experiments were performed according to the manufacturer's protocol.

SELEX of Assembled Zinc Finger Proteins

A template oligonucleotide was designed to contain a random 20-nucleotide region flanked, on both sides, by invariant sequences. In addition, two primers that were complementary to the invariant regions of the template oligonucleotide were designed for the PCR amplification. The template oligonucleotide was converted to double-stranded DNA by Klenow fragment extension from one of the primers. For enrichment of the target sequences bound by zinc finger proteins, 100 μg of the GST-fusion proteins was mixed with 10 pmol of double-stranded template DNA in 100 μl of binding buffer (25 mM Hepes pH 7.9, 40 mM KCl, 3 mM MgCl₂, 1 mM DTT) for one hour at room temperature. GST-resin (10 μl) was then added to the mixture. After incubation for 30 min at room temperature, the resin was washed three times with binding buffer containing 2.5% skim milk.

The bound double-stranded template oligomers were dissociated by incubating the resins with 100 μl of 1 M KCl for 10 min at room temperature. After PCR amplification of the rescued double-stranded template oligomers, a new round of SELEX was repeated. This process was repeated eight times. The final PCR product was digested with XbaI and BamHI and inserted into pBLUESCRIPT™ KS digested with the same enzymes. The DNA sequences of at least eight individual inserts per zinc finger protein were determined.

### EXAMPLE 7

### Sequences of Exemplary Proteins

The following are the amino acid sequences of the DNA binding regions of exemplary proteins that can regulate VEGF-A:

TABLE 12

Amino Acid Sequences of DNA Binding
Domains of Exemplary Proteins

| | | SEQ |
|---|---|---|
| | | ID |
| Name | Amino Acid Sequence | NO: |
| F475 | YKCGQCGKFY SQVSHLTRHQ KIHTGEKPFQ CKTCQRKFSR | 20 |
| | SDHLKTHTRT HTGEKPYICR KCGRGFSRKS NLIRHQRTHT | |
| | GEK | |
| F121 | YKCEECGKAF RQSSHLTTHK IIHTGEKPYK CMECGKAFNR | 21 |
| | RSHLTRHQRI HTGEKPFQCK TCQRKFSRSD HLKTHTRTHT | |
| | GEK | |
| F435 | YKCGQCGKFY SQVSHLTRHQ KIHTGEKPFQ CKTCQRKFSR | 22 |
| | SDHLKTHTRT HTGEKPYKCM ECGKAFNRRS HLTRHQRIHT | |
| | GEK | |
| F547 | YKCMECGKAF NRRSHLTRHQ RIHTGEKPFQ CKTCQRKFSR | 23 |
| | SDHLKTHTRT HTGEKPYECD HCGKAFSVSS NLNVHRRIHT | |
| | GEK | |
| F2825 | YECDHCGKSF SQSSHLNVHK RTHTGEKPFL CQYCAQRFGR | 24 |
| | KDHLTRHMKK SHTGEKPFQC KTCQRKFSRS DHLKTHTRTH | |
| | TGEK | |
| F480 | YKCMECGKAF NRRSHLTRHQ RIHTGEKPFQ CKTCQRKFSR | 25 |
| | SDHLKTHTRT HTGEKPYKCM ECGKAFNRRS HLTRHQRIHT | |
| | GEK | |
| F2828 | YKCKQCGKAF GCPSNLRRHG RTHTGEKPYR CEECGKAFRW | 26 |
| | PSNLTRHKRI HTGEKPFLCQ YCAQRFGRKD HLTRHMKKSH | |
| | TGEK | |
| F625 | YKCKQCGKAF GCPSNLRRHG RTHTGEKPYR CEECGKAFRW | 27 |
| | PSNLTRHKRI HTGEKPYKCM ECGKAFNRRS HLTRHQRIHT | |
| | GEK | |
| F2830 | YRCKYCDRSF SDSSNLQRHV RNIHTGEKPY RCEECGKAFR | 28 |
| | WPSNLTRHKR IHTGEKPFLC QYCAQRFGRK DHLTRHMKKS | |
| | HTGEK | |
| F2838 | YRCKYCDRSF SDSSNLQRHV RNIHTGEKPY RCEECGKAFR | 29 |
| | WPSNLTRHKR IHTGEKPYKC MECGKAFNRR SHLTRHQRIH | |
| | TGEK | |
| F2604 | YSCGICGKSF SDSSAKRRHC ILHTGEKPYI CRKCGRGFSR | 30 |
| | KSNLIRHQRT HTGEKPFQCK TCQRKFSRSD HLKTHTRTHT | |
| | GEKPYTCKQC GKAFSVSSSL RRHETTHTGE K | |
| F2605 | YKCEECGKAF RQSSHLTTHK IIHTGEKPYS CGICGKSFSD | 31 |
| | SSAKRRHCIL HTGEKPYICR KCGRGFSRKS NLIRHQRTHT | |
| | GEKPFQCKTC QRKFSRSDHL KTHTRTHTGE K | |
| F2607 | FQCKTCQRKF SRSDHLKTHT RTHTGEKPYE CDHCGKAFSV | 32 |
| | SSNLNVHRRI HTGEKPYKCE ECGKAFRQSS HLTTHKIIHT | |
| | GEKPYSCGIC GKSFSDSSAK RRHCILHTGE K | |
| F2615 | YKCMECGKAF NRRSHLTRHQ RIHTGEKPYT CSDCGKAFRD | 33 |
| | KSCLNRHRRT HTGEKPYKCE ECGKAFRQSS HLTTHKIIHT | |
| | GEKPYTCSDC GKAFRDKSCL NRHRRTHTGE K | |
| F2633 | YECEKCGKAF NQSSNLTRHK KSHTGEKPYK CGQCGKFYSQ | 34 |
| | VSHLTRHQKI HTGEKPFQCK TCQRKFSRSD HLKTHTRTHT | |
| | GEKPYICRKC GRGFSRKSNL IRHQRTHTGE K | |
| F2634 | YKCKQCGKAF GCPSNLRRHG RTHTGEKPFQ CKTCQRKFSR | 35 |
| | SDHLKTHTRT HTGEKPYICR KCGRGFSRKS NLIRHQRTHT | |
| | GEKPYKCMEC GKAFNRRSHL TRHQRIHTGE K | |
| F2636 | YKCMECGKAF NRRSHLTRHQ RIHTGEKPYK CEECGKAFRQ | 36 |
| | SSHLTTHKII HTGEKPYKCM ECGKAFNRRS HLTRHQRIHT | |
| | GEKPYVCDVE GCTWKFARSD ELNRHKKRHT GEK | |
| F2644 | YECEKCGKAF NQSSNLTRHK KSHTGEKPYK CMECGKAFNR | 37 |
| | RSHLTRHQRI HTGEKPYKCP DCGKSFSQSS SLIRHQRTHT | |
| | GEKPYKCMEC GKAFNRRSHL TRHQRIHTGE K | |
| F2646 | YKCEECGKAF RQSSHLTTHK IIHTGEKPYT CSDCGKAFRD | 38 |
| | KSCLNRHRRT HTGEKPFQCK TCQRKFSRSD HLKTHTRTHT | |
| | GEKPYKCKQC GKAFGCPSNL RRHGRTHTGE K | |
| F2650 | YKCEECGKAF RQSSHLTTHK IIHTGEKPYR CEECGKAFRW | 39 |
| | PSNLTRHKRI HTGEKPYKCM ECGKAFNRRS HLTRHQRIHT | |
| | GEKPYRCEEC GKAFRWPSNL TRHKRIHTGE K | |
| F2679 | YECDHCGKAF SVSSNLNVHR RIHTGEKPYK CMECGKAFNR | 40 |
| | RSHLTRHQRI HTGEKPYVCD VEGCTWKFAR SDELNRHKKR | |
| | HTGEKPYVCS KCGKAFTQSS NLTVHQKIHT GEK | |
| F2610 | YICRKCGRGF SRKSNLIRHQ RTHTGEKPYK CMECGKAFNR | 41 |
| | RSHLTRHQRI HTGEKPFQCK TCQRKFSRSD HLKTRTRTHT | |
| | GEKPYKCMEC GKAFNRRSHL TRHQRIHTGE K | |
| F2612 | YKCMECGKAF NRRSHLTRHQ RIHTGEKPFQ CKTCQRKFSR | 42 |
| | SDHLKTHTRT HTGEKPYKCM ECGKAFNRRS HLTRHQRIHT | |
| | GEKPFQCKTC QRKFSRSDHL KTHTRTHTGE K | |
| F2638 | YICRKCGRGF SRKSNLIRHQ RTHTGEKPYK CGQCGKFYSQ | 43 |
| | VSHLTRHQKI HTGEKPFQCK TCQRKFSRSD HLKTHTRTHT | |
| | GEKPYKCMEC GKAFNRRSHL TRHQRIHTGE K | |
| F109 | YVCDVEGCTW KFARSDELNR HKKRHTGEKP YKCPDCGKSF | 44 |
| | SQSSSLIRHQ RTHTGEKPYK CEECGKAFRQ SSHLTTHKII | |
| | HTGEKPYICR KCGRGFSRKS NLIRHQRTHT GEK | |
| F2608 | YKCMECGKAF NRRSHLTRHQ RIHTGEKPFQ CKTCQRKFSR | 45 |
| | SDHLKTHTRT HTGEKPYECD HCGKAFSVSS NLNVHRRIHT | |
| | GEKPYKCEEC GKAFRQSSHL TTHKIIHTGE K | |
| F2611 | YKCMECGKAF NRRSHLTRHQ RIHTGEKPYK CMECGKAFNR | 46 |
| | RSHLTRHQRI HTGEKPYRCE ECGKAFRWPS NLTRHKRIHT | |
| | GEKPYKCMEC GKAFNRRSHL TRHQRIHTGE K | |
| F2617 | YVCDVEGCTW KFARSDELNR HKKRHTGEKP YKCMECGKAF | 47 |
| | NRRSHLTRHQ RIHTGEKPYT CSDCGKAFRD KSCLNRHRRT | |
| | HTGEKPYKCE ECGKAFRQSS HLTTHKIIHT GEK | |
| F2619 | YKCMECGKAF NRRSHLTRHQ RIHTGEKPYE CNYCGKTFSV | 48 |
| | SSTLIRHQRI HTGEKPYECE KCGKAFNQSS NLTRHKKSHT | |
| | GEKPFQCKTC QRKFSRSDHL KTHTRTHTGE K | |
| F2623 | YKCEECGKAF RQSSHLTTHK IIHTGEKPYI CRKCGRGFSR | 49 |
| | KSNLIRHQRT HTGEKPYRCE ECGKAFRWPS NLTRHKRIHT | |
| | GEKPYVCDVE GCTWKFARSD ELNRHKKRHT GEK | |
| F2625 | YKCEECGKAF RQSSHLTTHK IIHTGEKPYR CEECGKAFRW | 50 |
| | PSNLTRHKRI HTGEKPFQCK TCQRKFSRSD HLKTHTRTHT | |
| | GEKPYVCDVE GCTWKFARSD ELNRHKKRHT GEK | |
| F2628 | YTCKQCGKAF SVSSSLRRHE TTHTGEKPYR CEECGKAFRW | 51 |
| | PSNLTRHKRI HTGEKPYICR KCGRGFSRKS NLIRHQRTHT | |
| | GEKPYTCKQC GKAFSVSSSL RRHETTHTGE K | |
| F2629 | YKCGQCGKFY SQVSHLTRHQ KIHTGEKPYT CKQCGKAFSV | 52 |
| | SSSLRRHETT HTGEKPYRCE ECGKAFRWPS NLTRHKRIHT | |
| | GEKPYICRKC GRGFSRKSNL IRHQRTHTGE K | |
| F2630 | YVCDVEGCTW KFARSDELNR HKKRHTGEKP YKCGQCGKFY | 53 |
| | SQVSHLTRHQ KIHTGEKPYT CKQCGKAFSV SSSLRRHETT | |
| | HTGEKPYRCE ECGKAFRWPS NLTRHKRIHT GEK | |
| F2635 | YKCGQCGKFY SQVSHLTRHQ KIHTGEKPYI CRKCGRGFSR | 55 |
| | KSNLIRHQRT HTGEKPYKCG QCGKFYSQVS HLTRHQKIHT | |
| | GEKPFQCKTC QRKFSRSDHL KTHTRTHTGE K | |
| F2637 | FQCKTCQRKF SRSDHLKTHT RTHTGEKPYI CRKCGRGFSR | 56 |
| | KSNLIRHQRT HTGEKPYKCM ECGKAFNRRS HLTRHQRIHT | |
| | GEKPYRCEEC GKAFRWPSNL TRHKRIHTGE K | |
| F2642 | FQCKTCQRKF SRSDHLKTHT RTHTGEKPYK CMECGKAFNR | 57 |
| | RSHLTRHQRI HTGEKPYKCK QCGKAFGCPS NLRRHGRTHT | |
| | GEKPFQCKTC QRKFSRSDHL KTHTRTHTGE K | |
| F2643 | YKCMECGKAF NRRSHLTRHQ RIHTGEKPYK CKQCGKAFGC | 58 |
| | PSNLRRHGRT HTGEKPFQCK TCQRKFSRSD HLKTHTRTHT | |
| | GEKPYKCKQC GKAFGCPSNL RRHGRTHTGE K | |
| F2648 | YKCPDCGKSF SQSSSLIRHQ RTHTGEKPYK CGQCGKFYSQ | 59 |
| | VSHLTRHQKI HTGEKPYICR KCGRGFSRKS NLIRHQRTHT | |
| | GEKPYICRKC GRGFSRKSNL IRHQRTHTGE K | |
| F2651 | YECNYCGKTF SVSSTLIRHQ RIHTGEKPYK CEECGKAFRQ | 60 |
| | SSHLTTHKII HTGEKPYRCE ECGKAFRWPS NLTRHKRIHT | |
| | GEKPYKCMEC GKAFNRRSHL TRHQRIHTGE K | |
| F2653 | YECNYCGKTF SVSSTLIRHQ RIHTGEKPYE CEKCGKAFNQ | 63. |
| | SSNLTRHKKS HTGEKPYKCM ECGKAFNRRS HLTRHQRIHT | |
| | GEKPYECEKC GKAFNQSSNL TRHKKSHTGE K | |
| F2654 | YECEKCGKAF NQSSNLTRHK KSHTGEKPYK CMECGKAFNR | 62 |
| | RSHLTRHQRI HTGEKPYECE KCGKAFNQSS NLTRHKKSHT | |
| | GEKPYECDHC GKAFSVSSNL NVHRRIHTGE K | |
| F2662 | YTCSDCGKAF RDKSCLNRHR RTHTGEKPFQ CKTCQRKFSR | 63 |
| | SDHLKTHTRT HTGEKPYECN YCGKTFSVSS TLIRHQRIHT | |
| | GEKPYVCDVE GCTWKFARSD ELNRHKKRHT GEK | |
| F2667 | YKCMECGKAF NRRSHLTRHQ RIHTGEKPYT CSDCGKAFRD | 64 |
| | KSCLNRHRRT HTGEKPFQCK TCQRKFSRSD HLKTHTRTHT | |
| | GEKPYKCMEC GKAFNRRSHL TRHQRIHTGE K | |
| F2668 | YKCMECGKAF NRRSHLTRHQ RIHTGEKPYK CMECGKAFNR | 65 |
| | RSHLTRHQRI HTGEKPYVCS KCGKAFTQSS NLTVHQKIHT | |
| | GEKPYVCSKC GKAFTQSSNL TVHQKIHTGE K | |
| F2673 | FQCKTCQRKF SRSDHLKTHT RTHTGEKPYT CKQCGKAFSV | 66 |
| | SSSLRRHETT HTGEKPYVCD VEGCTWKFAR SDELNRHKKR | |
| | HTGEKPYKCP DCGKSFSQSS SLIRHQRTHT GEK | |
| F2682 | YICRKCGRGF SRKSNLIRHQ RTHTGEKPYK CPDCGKSFSQ | 67 |
| | SSSLIRHQRT HTGEKPYECE KCGKAFNQSS NLTRHKKSHT | |
| | GEKPYKCMEC GKAFNRRSHL TRHQRIHTGE K | |
| F2689 | YICRKCGRGF SRKSNLIRHQ RTHTGEKPYS CGICGKSFSD | 68 |
| | SSAKRRHCIL HTGEKPYECE KCGKAFNQSS NLTRHKKSHT | |
| | GEKPYKCEEC GKAFRQSSHL TTHKIIHTGE K | |
| F2697 | YKCMECGKAF NRRSHLTRHQ RIHTGEKPYK CKQCGKAFGC | 69 |
| | PSNLRRHGRT HTGEKPYKCE ECGKAFRQSS HLTTHKIIHT | |
| | GEKPYICRKC GRGFSRKSNL IRHQRTHTGE K | |
| F2699 | YICRKCGRGF SRKSNLIRHQ RTHTGEKPYK CEECGKAFRQ | 70 |
| | SSHLTTHKII HTGEKPYSCG ICGKSFSDSS AKRRHCILHT | |
| | GEKPYKCMEC GKAFNRRSHL TRHQRIHTGE K | |
| F2703 | YKCGQCGKFY SQVSHLTRHQ KIHTGEKPYK CMECGKAFNR | 71 |
| | RSHLTRHQRI HTGEKPYVCD VEGCTWKFAR SDELNRHKKR | |
| | HTGEKPYKCM ECGKAFNRRS HLTRHQRIHT GEK | |
| F2702 | YKCMECGKAF NRRSHLTRHQ RIHTGEKPYK CGQCGKFYSQ | 54 |
| | VSHLTRHQKI HTGEKPYKCM ECGKAFNRRS HLTRHQRIHT | |
| | GEKPYVCSKC GKAFTQSSNL TVHQKIHTGE K | |

A polypeptide, e.g., that includes a sequence described above, also include a tag (e.g., the HA tag), a NLS, a linker, and a regulatory domain (e.g., an activation or repression domain). These elements can be arrange in any order, from N- to C-terminus. In one example, the polypeptide is arranged as follows: HA tag-NLS-PGEKP-DNA binding domain (e.g., a sequence described above)-AAA-p65. Or more particularly:

MVYPYDVPDYAELPPKKKRKVGIRIPGEKP-DNA_BIDING_DOMAIN-AAA-p65; (wherein the leader N-terminal to the DNA binding domain is SEQ ID NO:126)
YPYDVPDYA (3-12 of SEQ ID NO:126) is an exemplary tag (ere the HA-tag)
PPKKKRKV (15-21 of SEQ ID NO:126) is an exemplary NLS (Nuclear localization signal)
“ZFP” is an array of zinc finger domains

In another example, the polypeptide includes the DNA binding domain and a repression domain, e.g., a KRAB or KOX domain.

Nucleic acid encoding a polypeptide described in this example can be producing using any choice of codons, e.g., codons useful (e.g., optimized) for prokaryotic expression, codons useful (e.g., optimized) for eukaryotic expression, or codons that encode corresponding naturally occurring domains.

Results indicate that a number of zinc finger can activate VEGF-A production.

TABLE 13
VEGF-A Activation

| | Fold |
|---|---|
| ZFP ID | Activation |
| F109 | 3.5 |
| F121 | 4.4 |
| F435 | 12.5 |
| F475 | 11.1 |
| F480 | 9.2 |
| F625 | 9.0 |
| F2604 | 4.9 |
| F2605 | 10.9 |
| F2607 | 5.4 |
| F2608 | 2.1 |
| F2610 | 7.4 |
| F2612 | 6.3 |
| F2615 | 8.1 |
| F2617 | 2.3 |
| F2619 | 2.3 |
| F2623 | 2.3 |
| Irrelevant | 1.1 |
| ZFP | |
| F2625 | 2.1 |
| F2628 | 1.8 |
| F2629 | 3.8 |
| F2630 | 2.0 |
| F2633 | 11.9 |
| F2634 | 6.5 |
| F2635 | 2.8 |
| F2636 | 13.3 |
| F2638 | 5.7 |
| F2642 | 3.6 |
| F2643 | 3.6 |
| F2644 | 10.7 |
| F2646 | 10.2 |
| F2648 | 2.3 |
| F2650 | 12.6 |
| F2651 | 4.9 |
| Parental | 1.0 |
| vector P3 | |
| F2653 | 2.6 |
| F2654 | 2.3 |
| F2668 | 2.1 |
| F2673 | 3.1 |
| F2679 | 4.5 |
| F2682 | 3.4 |
| F2689 | 2.1 |
| F2697 | 1.9 |
| F2699 | 1.9 |
| F2702 | 3.1 |
| F2703 | 4.5 |
| F2825 | 2.8 |
| F2828 | 8.8 |
| F2830 | 6.8 |
| F2838 | 5.8 |

### EXAMPLE 8

### VEGF-A Production by an Encapsulated Cells

A nucleic acid construct that includes a coding region encoding the F435-p65 zinc finger protein operably linked to a doxycycline-inducible promoter was stably transfected into Flp-T-Rex293 cells.

Human embryonic kidney (HEK) cell lines stably expressing ZFP-TFs were generated as follows: Plasmids encoding ZFP-TFs were stably introduced into FlpTRex-293 cell lines (Invitrogen) essentially as described in the manufacturer's protocol. Briefly, the HindIII.XhoI fragment from the pLFD-p65 vectors, which contain DNA segments that encode ZFP-TFs, were subcloned into pcDNA5/FRT/TO (Invitrogen). The resulting plasmids were cotransfected along with pOG44 (Invitrogen) into Flp-In™ TRex™-293 cells to induce a site-specific integration event. Stable integrants were then screened. The resulting cell lines expressed zinc finger protein conditionally upon the addition of Doxycyclin (1 ug/ml) into culture medium. To immobilize the cells stably expressing F435-p65, Sodium Alginate (Sigma) was dissolved in PBS to a final concentration of 1% (wt/v) and gently mixed with cells to a final density of 10⁶ (cells/ml). The suspension of cells were added dropwise into CaCl₂ (100 mM) where cellular capsules were solidified for 15 min, then were washed in PBS. The encapsulated cells were added to culture medium. Expression was induced with 1 μg/ml doxycycline and the amount of VEGF-A produced by the encapsulated cells was measured. In one experiment with F435-p65, the cells (cell line #151) grown in the presence of doxycycline produced at least 600 pg/mL of VEGF-A after 2 days, at least 4000 pg/mL after three days, about 5000 pg/mL at four days, and at least 5300 pg/mL at five days. VEGF-A production was at least 5, 10, 50, or 100 fold greater than controls that did not include the F435-p65 zinc finger protein or cells that were not grown in the presence of doxycycline.

### EXAMPLE 9

### Cell-Based Assay for Human VEGF-A Expression

The 3×10⁴ HEK293T cells were transfected with 100 ng of each pLFD-4F-p65 plasmid in 96-culture plates precoated with poly-L-lysine (Biocoat). The culture supernatants were harvested at 48 hours post transfection and stored immediately at −80° C. until they were used. The transfection efficiency was estimated at a well of each plate transfected with 100 ng of lacZ, by staining with X-gal. The calculated transfection efficiencies varied in a range of 70-80% in each experiment.

The production of VEGF-A was analyzed by measuring secreted VEGF-A protein by sandwich ELISA. The capture antibody (AF-293-NA from R&D Systems), biotinylated detection antibody (BAF293 from R&D Systems) were purchased from R&D systems, streptavidin-AP (SA110) and substrate buffer (ES011) from Chemicon, substrate pNPP (N-9389) from Sigma Aldrich. The ELISA procedures were carried out with automated workstation (GENESIS RSP 150™, TECAN). The optical density (OD) at 405 nm was measured (POWERWAVE™ X340, BioTek Instrument Inc.) and the quantity of VEGF-A was calculated from standard curve obtained from the OD values of serially diluted recombinant human VEGF-A protein (R&D systems). Relative VEGF-A production was calculated by normalizing VEGF-A concentrations obtained from cultures individually transfected with pLFD-4F-p65 to that obtained from cultures transfected with the parental vector p3.

### EXAMPLE 10

### Cell-Based Assay for Human VEGF-A Expression

The zinc finger protein F121 consisted of three human zinc finger domains designed to bind 9 bp sequences of human VEGF promoter at about nucleotide +434 relative to the transcription initiation site of human VEGF-A gene; F109 consisted of four human zinc finger domains designed to bind a 12 bp sequence of human VEGF promoter at about the −536 nucleotide relative to the transcription initiation site of human VEGF-A gene; and F435 consisted of three human zinc finger domains designed to bind 9 bp sequences at the positions −90R and −391R (wherein R means reverse strand) of human VEGF-A gene.

Construction of Luciferase Reporter Plasmids Containing Human VEGF Promoter

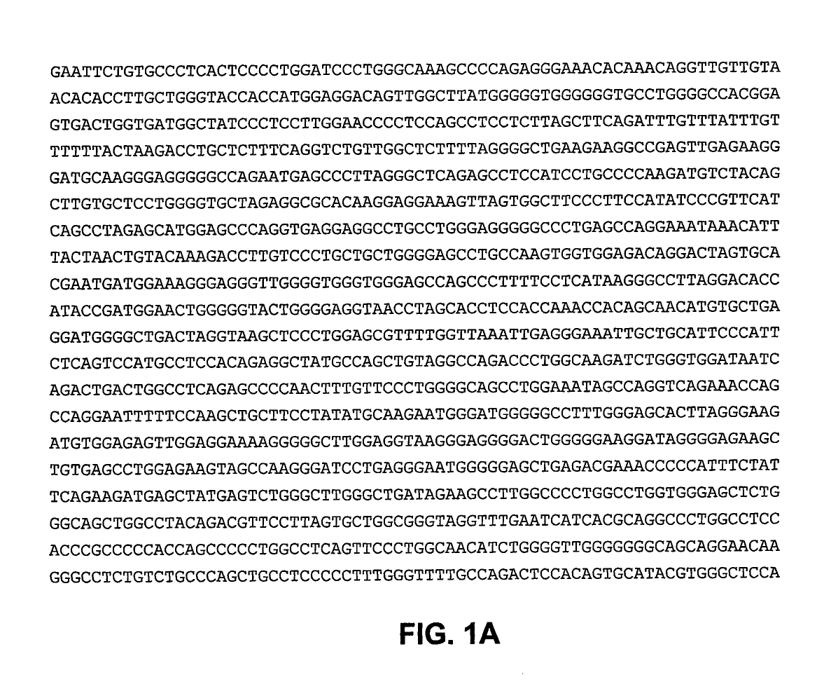

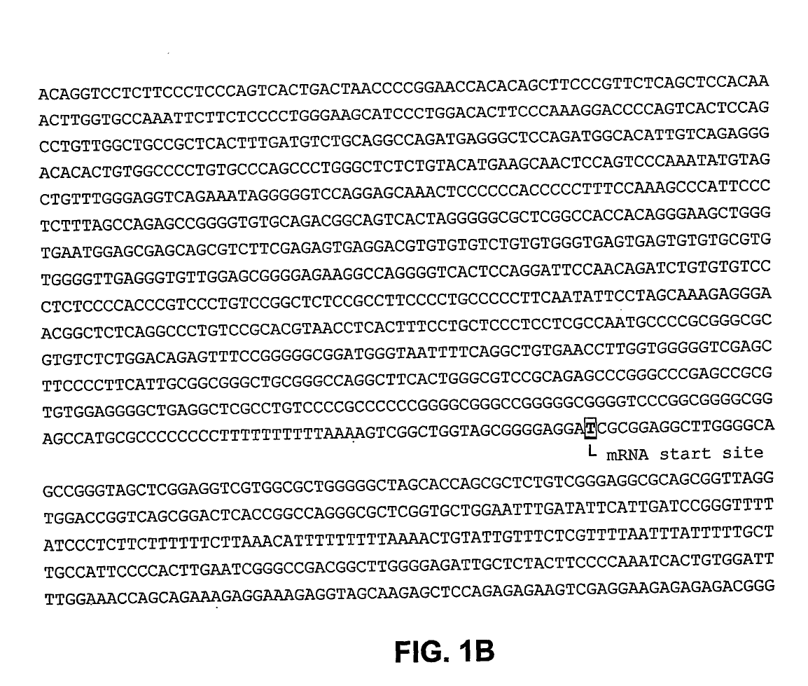

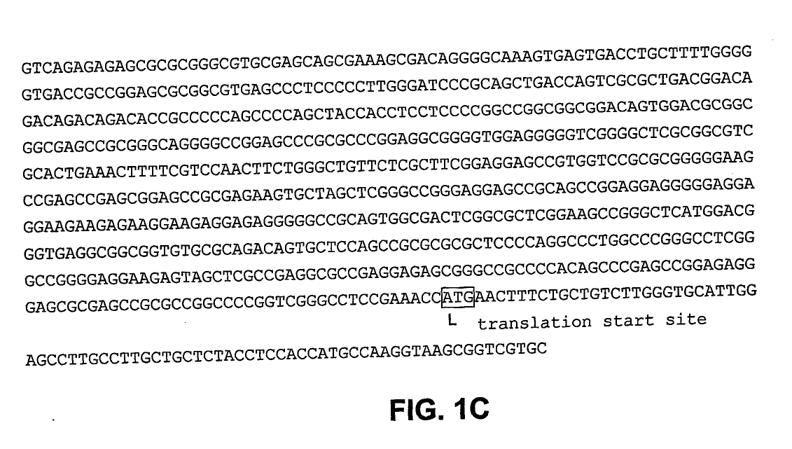

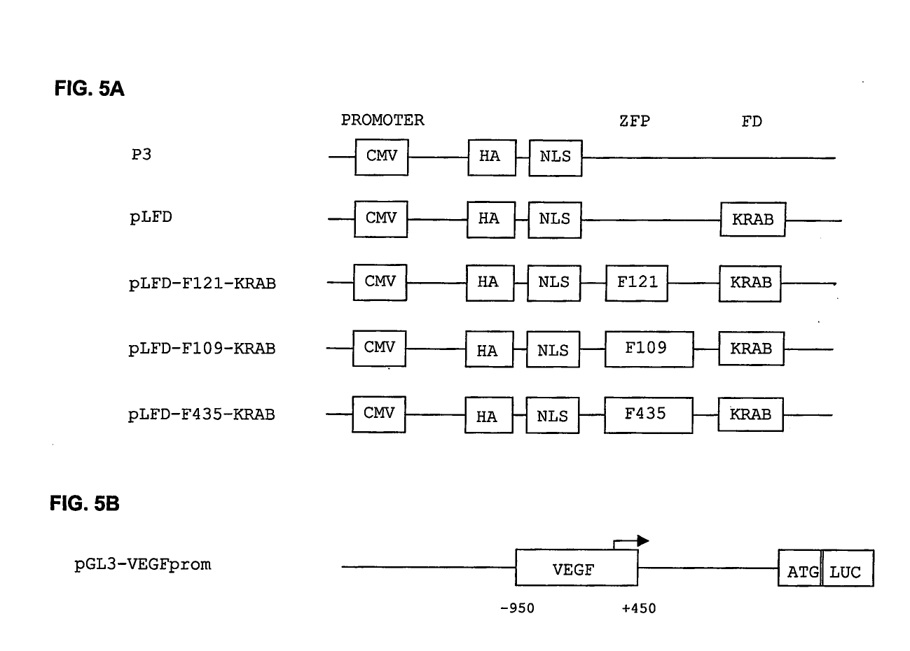

The native human VEGF promoter DNA (at position −950 to +450, numbering relative to the transcription initiation sequence shown in FIGS. 1A, B, C) was PCR-amplified from human genomic DNA using sequence specific primers and cloned into the KpnI/XhoI restriction site of plasmid pGL3(Promega, E1751), and the resulting plasmid was designated pGL3-VEGFprom (FIG. 5B).

Repression of the Luciferase Reporter Containing Native Human VEGF Promoter by Zinc Finger Protein

293 cells were transfected with luciferase reporter plasmid pGL3-VEGFprom containing native human VEGF promoter (−950 to +450 from the transcription initiation site) and 30 ng of pLFD-F121-KRAB or pLFD-F109-KRAB. Luciferase activity was measured as described. Fold repression values were calculated by normalizing the firefly luciferase activity against the renilla luciferase activity and the result was compared with that of the control wherein 293 cells were transfected with the control vector pLFD and the reporter plasmid.

The plasmids encoding F121-KRAB (30 ng) and F109-KRAB (30 ng) reduced the reporter activity 8.7 fold and 6.1 fold, respectively.

Repression of Endogenous VEGF-A mRNA Expression by ZFP-KRAB

ZFP expression plasmids were transfected into human embryonic kidney 293F cells (Gibco Life Technologies). 293F cells allow for high transfection efficiencies.

293F cells were precultured in the wells of a 24-well culture plate, at a density of 10⁵ cells/well, in 1 ml of DMEM supplemented with 10% FBS for 24 h in a humid atmosphere containing 5% CO₂ at 37° C. The cells were transfected with 0, 200, or 400 ng of plasmids encoding chimeric zinc finger proteins of interest using a LIPOFECTAMINE PLUS™ (Life Technologies). The total amount of DNA was adjusted to 400 ng by adding the parental vector as a control if less than 400 ng of the zinc finger protein expression vector was used. The cells were further incubated for 48 hours. The total RNA was extracted from the cells with the TRIZOL® reagent (Gibco Life Technologies).

Quantification of VEGF mRNA was Carried Out by the Following Real Time RT-PCR.

The reverse transcription reactions were performed with 4 μg of the total RNA using oligo-dT as the first-strand synthesis primer for mRNA, dNTP and MMLV reverse transcriptase provided in the Superscript first-strand synthesis system (Gibco Life Technologies) to obtain a first-strand cDNA. To analyze mRNA quantities, 1 μl of the first-strand cDNA thus obtained was amplified by real time PCR using VEGF-A cDNA specific primers (Forward primer 5′-CGGGGTACCCCCTCCCAGTCACTGACTAAC-3′, SEQ ID NO:127) and (Reverse primer 5′-CCGCTCGAGTCCGGCGGTCACCCCCAAAAG-3′; SEQ ID NO:128). Since this method is sensitive to the initial amount of RNA, the initial RNA amounts were normalized to the GAPDH mRNA quantities calculated by specific amplification using GAPDH-specific primers. The amplification of VEGF- and GAPDH-specific cDNAs was monitored and analyzed in real-time with a QUANTITECT SYBR™ kit (QIAGEN, Valencia, Calif.) and ROTORGENE™ 2000 real-time cycler (Corbett, Sydney, Australia), and the cDNAs were quantified by serial dilution of the standards included in the reactions.

Repression of VEGF-A mRNA Synthesis by Zinc Finger Proteins

The expression of endogenous VEGF-A mRNA was reduced 2.2 fold (54.5% repression, 200 ng pLFD-F435-KRAB) and 4.1 fold (75.6% repression, 400 ng pLFD-F435-KRAB) relative to untreated control cells. These results show a dose dependant effect.

Repression of VEGF-A Protein Production by ZFP (F435-KRAB)

In order to examine whether the repression of VEGF-A mRNA expression resulted in the reduction of VEGF-A protein secretion, 293F cells (10⁴/96 well plate) were transfected with 0 to 200 ng of ZFP expression plasmids (pLFD-F435-KRAB) and cultured for 72 hours. VEGF protein that accumulated in the culture medium (100 ul each) and dilutions of a recombinant human VEGF-A protein standards were analyzed using sandwich ELISA (enzyme linked immunosorbent assay), wherein the supernatant of culture was reacted with a anti-human VEGF antibody (R&D systems; AF-293-NA), biotinylated anti-human VEGF antibody (R&D systems; BAF293), streptavidin-alkaline phosphatase. The antigen-antibody complex was reacted with pNPP (p-Nitrophenyl phosphate) dissolved in pNPP buffer (Chemicon; ES011). VEGF-A concentrations in the samples were determined from the absorbance at 405 nm which was measured with POWERWAVE™ X340(Bio-TEK Instrument Inc., Winooski Vt.).

F435-KRAB reduced VEGF-A production in a dose dependant manner. When 200 ng of the plasmid was used VEGF-A protein concentration was repressed 3.9 fold (138 pg/ml) relative to control cells transfected with a control plasmid, pLFD-F435-KRAB 200 ng. See Table 14.

TABLE 14
Titration of F435-KRAB

| | Concentration of F435-KRAB plasmid (ng) | | | | |
|---|---|---|---|---|---|
| | | | | | Control |
| | 25 | 50 | 100 | 200 | (200 ng) |
| VEGF-A | 420 ± 98 | 345 ± 50 | 172 ± 13 | 138 ± 14 | 536 ± 14 |
| (pg/ml) | | | | | |
| Fold | 1.3 | 1.6 | 3.1 | 3.9 | 1.0 |
| Repression | | | | | |

Repression of VEGF-A Gene Induction by Hypoxic Conditions

VEGF-A gene is known as a crucial factor for inducing angiogenesis. VEGF-A activity is essential for the development and growth of many tumors. VEGF-A activity has been found to be stimulated by hypoxia condition in cancer tissues. A high level of VEGF-A expression is frequently observed in tumor cells.

When the medium for culturing 293F cells is treated with 100 to 800 μM of CoCl₂ for about 7 hours, a hypoxia condition is induced and VEGF production by cells is rapidly escalated. The following experiment was carried out in order to examine whether the zinc finger protein can inhibit the VEFG expression in the hypoxia condition.

293F cells (10⁴ cells/well, 96-well plate) were transfected with pLFD-F435-KRAB 50 ng and incubated for 48 hours. In order to induce the hypoxic condition, 800 μM of CoCl₂ was added to the medium at the last 7 hours stage of the culture. The amount of VEGF-A secreted in the culture medium was determined by ELISA.

VEGF production from the hypoxic CoCl₂ treated culture with mock-transfected cells increased to about 1,039 pg/ml, in contrast to about 273 pg/ml in the untreated control cells. This observation confirms that hypoxia strongly induces VEGF-A production. However, cells transfected with pLFD-F435-KRAB did not induce VEGF-A production in hypoxic conditions. These cells produced only about 272 pg/ml of VEGF-A, a concentration similar to the non-hypoxic control. This results demonstrates that expression of F435-KRAB inhibits VEGF-A production under hypoxic conditions. Since the transfection rate was only about 85-90%, it is possible that the residual level of VEGF-A production is due to the untransfected cells in the culture. We concluded that F435-KRAB and similarly functional chimeric zinc finger proteins are potent repressors of VEGF-A expression.

The selected zinc finger proteins or related proteins that include domains with the same motifs may be used, e.g., as therapeutic agents. Such agents can be, e.g., to repress VEGF-A expression and thereby retard the growth of tumor cells.

A number of embodiments of the invention have been described. Nevertheless, it will be understood that various modifications may be made without departing from the spirit and scope of the invention. Accordingly, other embodiments are within the scope of the following claims.

What is claimed is:

1. A polypeptide comprising a DNA binding domain that includes a plurality of zinc finger domains, wherein
the DNA binding domain can bind to a site in a VEGF gene, and
at least two of the zinc finger domains each include respective zinc finger domain motifs listed in column 2 of Table 1, Table 2, Table 3, Table 4 or Table 5.

2. The polypeptide of claim 1, wherein the zinc finger domains each include respective zinc finger domain motifs listed in column 2 of Table 1 or Table 3.

3. The polypeptide of claim 2, wherein the zinc finger domain is selected from the group consisting of the zinc finger domains listed in column 3 of Table 1 or Table 3.

4. The polypeptide of claim 3, wherein the DNA binding domain includes, in N-terminal to C-terminal order, first, second and third zinc finger domains, wherein
(1) the DNA contacting residues at positions −1, 2, 3, and 6 of first zinc finger domain are QSHR, those of the second zinc finger domain are RDHT, and those of the third zinc finger domain are RSX₁R, wherein X₁ is H or N;
(2) the DNA contacting residues at positions −1, 2, 3, and 6 of first zinc finger domain are QSHX₂, those of the second zinc finger domain are RX₃HR, and those of the third zinc finger domain are RDHT, wherein X₂ is T or V and X₃ is S or D;
(3) the DNA contacting residues at positions −1, 2, 3, and 6 of first zinc finger domain are RSHR, those of the second zinc finger domain are RDHT, and those of the third zinc finger domain are VSNV;
(4) the DNA contacting residues at positions −1, 2, 3, and 6 of first zinc finger domain are RDER, those of the second zinc finger domain are QSSR, and those of the third zinc finger domain are QSHT;
(5) the DNA contacting residues at positions −1, 2, 3, and 6 of first zinc finger domain are QSSR, those of the second zinc finger domain are QSHT, and those of the third zinc finger domain are RSNR;
(6) the DNA contacting residues at positions −1, 2, 3, and 6 of first zinc finger domain are QSNR, those of the second zinc finger domain are QSHR, and those of the third zinc finger domain are RDHT;
(7) the DNA contacting residues at positions −1, 2, 3, and 6 of first zinc finger domain are QSHR, those of the second zinc finger domain are RDHT, and those of the third zinc finger domain are RSNR;
(8) the DNA contacting residues at positions −1, 2, 3, and 6 of first zinc finger domain are RSHR, those of the second zinc finger domain are QSHT, and those of the third zinc finger domain are RSHR;
(9) the DNA contacting residues at positions −1, 2, 3, and 6 of first zinc finger domain are QSHT, those of the second zinc finger domain are RSHR, and those of the third zinc finger domain are RDER;
(10) the DNA contacting residues at positions −1, 2, 3, and 6 of first zinc finger domain are QSNR, those of the second zinc finger domain are RSHR, and those of the third zinc finger domain are QSSR;
(11) the DNA contacting residues at positions −1, 2, 3, and 6 of first zinc finger domain are RSHR, those of the second zinc finger domain are QSSR, and those of the third zinc finger domain are RSHR;
(12) the DNA contacting residues at positions −1, 2, 3, and 6 of first zinc finger domain are QSHT, those of the second zinc finger domain are WSNR, and those of the third zinc finger domain are RSHR; or
(13) the DNA contacting residues at positions −1, 2, 3, and 6 of first zinc finger domain are WSNR, those of the second zinc finger domain are RSHR, and those of the third zinc finger domain are WSNR.

5. The polypeptide of claim 2, wherein the zinc finger domain is selected from the group consisting of the zinc finger domains listed in column 3 of Table 2, Table 4 or Table 5.

6. The polypeptide of claim 1, wherein the VEGF gene is the human VEGF-A gene.

7. The polypeptide of claim 1, which regulates the VEGF gene expression.

8. The polypeptide of claim 1, wherein the polypeptide further comprises a transcription activation domain, a transcription repression domain, or a protein transduction domain.

9. The polypeptide of claim 8, wherein the transcription activation domain comprises p65 or VP16 activation domain.

10. The polypeptide of claim 8, wherein the transcription repression domain comprises Kid or KOX repression domain.

11. The polypeptide of claim 8, wherein the protein transduction domain is a part of TAT protein, VP22 protein, or Antennapedia homeodomain.

12. A nucleic acid that comprises a sequence encoding the polypeptide of claim 1.

13. The nucleic acid of claim 12, which comprises a sequence encoding the polypeptide of claim 8.

14. A modified mammalian cell that contains the polypeptide of claim 1.

15. The cell of claim 14, wherein the polypeptide is produced from a nucleic acid of claim 14 in the cell.

16. A pharmaceutical composition for preventing or treating a neoplastic disorder, an inflammatory disorder, or an angiogenesis-based disorder, which comprises the polypeptide of claim 1, the nucleic acid of claim 12 or the modified mammalian cell of claim 14, and a pharmaceutically acceptable carrier.

17. The pharmaceutical composition of claim 16, wherein the neoplastic disorder is a cancer.

18. The pharmaceutical composition of claim 16, wherein the zinc finger domain included in the polypeptide is selected from the group consisting of the zinc finger domains listed in column 3 of Table 1 or Table 3.

19. The pharmaceutical composition of claim 18, wherein the polypeptide comprises a DNA binding domain that includes, in N-terminal to C-terminal order, first, second and third zinc finger domains, wherein
(1) the DNA contacting residues at positions −1, 2, 3, and 6 of first zinc finger domain are QSHR, those of the second zinc finger domain are RDHT, and those of the third zinc finger domain are RSX₁R, wherein X₁ is H or N;
(2) the DNA contacting residues at positions −1, 2, 3, and 6 of first zinc finger domain are QSHX₂, those of the second zinc finger domain are RX₃HR, and those of the third zinc finger domain are RDHT, wherein X₂ is T or V and X₃ is S or D;
(3) the DNA contacting residues at positions −1, 2, 3, and 6 of first zinc finger domain are RSHR, those of the second zinc finger domain are RDHT, and those of the third zinc finger domain are VSNV;
(4) the DNA contacting residues at positions −1, 2, 3, and 6 of first zinc finger domain are RDER, those of the second zinc finger domain are QSSR, and those of the third zinc finger domain are QSHT;
(5) the DNA contacting residues at positions −1, 2, 3, and 6 of first zinc finger domain are QSSR, those of the second zinc finger domain are QSHT, and those of the third zinc finger domain are RSNR;
(6) the DNA contacting residues at positions −1, 2, 3, and 6 of first zinc finger domain are QSNR, those of the second zinc finger domain are QSHR, and those of the third zinc finger domain are RDHT;
(7) the DNA contacting residues at positions −1, 2, 3, and 6 of first zinc finger domain are QSHR, those of the second zinc finger domain are RDHT, and those of the third zinc finger domain are RSNR;
(8) the DNA contacting residues at positions −1, 2, 3, and 6 of first zinc finger domain are RSHR, those of the second zinc finger domain are QSHT, and those of the third zinc finger domain are RSHR;
(9) the DNA contacting residues at positions −1, 2, 3, and 6 of first zinc finger domain are QSHT, those of the second zinc finger domain are RSHR, and those of the third zinc finger domain are RDER;
(10) the DNA contacting residues at positions −1, 2, 3, and 6 of first zinc finger domain are QSNR, those of the second zinc finger domain are RSHR, and those of the third zinc finger domain are QSSR;
(11) the DNA contacting residues at positions −1, 2, 3, and 6 of first zinc finger domain are RSHR, those of the second zinc finger domain are QSSR, and those of the third zinc finger domain are RSHR;
(12) the DNA contacting residues at positions −1, 2, 3, and 6 of first zinc finger domain are QSHT, those of the second zinc finger domain are WSNR, and those of the third zinc finger domain are RSHR; or
(13) the DNA contacting residues at positions −1, 2, 3, and 6 of first zinc finger domain are WSNR, those of the second zinc finger domain are RSHR, and those of the third zinc finger domain are WSNR.

20. An encapsulated composition comprising
an encapsulation layer composed of a biocompatible material that is permeable to proteins having a molecular weight of at least 10 kDa, and
recombinant mammalian cells, wherein the cells contain a nucleic acid comprising a sequence encoding a chimeric zinc finger protein that regulates production of a secreted factor.

21. The encapsulated composition of claim 20, wherein the secreted factor is insulin, an insulin-like growth factor, VEGF, HGF, interferon, interleukin, or a fibroblast growth factor.

22. A method of regulating VEGF gene expression, which comprises
introducing the polypeptide of claim 1 or the nucleic acid of claim 12 into a cell.

23. The method of claim 22, wherein the polypeptide comprises a transcription activation domain, and VEGF gene expression is increased in the cell.

24. The method of claim 22, wherein the polypeptide comprises a transcription repression domain, and VEGF gene expression is decreased in the cell.

25. The method of claim 22, wherein the VEGF gene is human VEGF-A gene.

26. The method of claim 22, wherein the cell is a mammalian cell.

27. The method of claim 26, wherein the cell is a human cell.

28. A method of modulating angiogenesis in a subject, which comprises
administering the composition of claim 16 to the subject in an amount effective to reduce angiogenesis in the subject.

29. The method of claim 28, wherein the subject is a human that has or is suspected of having a cancer.
